(19) United States
(12) Patent Application Publication
(10) Pub. No.: US 2023/0405078 A1
(43) Pub. Date: Dec. 21, 2023

(54) DETECTION AND TREATMENT OF INTESTINAL FIBROSIS

(71) Applicant: THE REGENTS OF THE UNIVERSITY OF CALIFORNIA, OAKLAND, CA (US)

(72) Inventors: Hon Wai Koon, LOS ANGELES, CA (US); Charalabos Pothoulakis, LOS ANGELES, CA (US)

(73) Assignee: THE REGENTS OF THE UNIVERSITY OF CALIFORNIA, OAKLAND, CA (US)

(21) Appl. No.: 17/907,220

(22) PCT Filed: Mar. 26, 2021
(86) PCT No.: PCT/US2021/070321

§ 371 Date: Sep. 23, 2022

Related U.S. Application Data
(60) Provisional application No. 63/000,021, filed on Mar. 26, 2020.

(51) Int. Cl. A61K 38/00; A61P 37/02; A61P 3/00; G16H 20/10

(52) U.S. Cl. CPC A61K 38/005; A61P 37/02; A61P 3/00; G16H 20/10

(57) ABSTRACT

Methods and materials for measuring semm elafin levels, elevated levels of which have been confirmed to detect the presence of stricture in patients with Crohn's disease (CD), and a method for improving the accuracy of detecting the presence of stricture through the use of an algorithm developed through machine learning and/or through the use of clinical data. Further, materials and methods for treating intestinal stricture in a subject having Crohn's disease, as well as for inhibiting intestinal fibrosis, inflammatory bowel disease (IBD), metabolic disease, or obesity in a subject, comprises administering elafin to the subject.

This application claims benefit of U.S. provisional patent application No. 63/000,021, filed Mar. 26, 2020, the entire contents of which are incorporated by reference into this application.

### ACKNOWLEDGEMENT OF GOVERNMENT SUPPORT

This invention was made with government support under Grant Numbers DK084256 and DK103964, awarded by the National Institutes of Health. The government has certain rights in the invention.

### REFERENCE TO A SEQUENCE LISTING SUBMITTED VIA EFS-WEB

The content of the ASCII text file of the sequence listing named “UCLA259_seq” which is 2 kb in size was created on Mar. 25, 2021 and electronically submitted via EFS-Web herewith the application is incorporated herein by reference in its entirety.

### BACKGROUND

Intestinal stricture formation is a debilitating complication of inflammatory bowel disease (IBD). Currently, there is no effective approach to prevent or reverse the development of intestinal fibrosis. Anti-inflammatory agents have little to no effect on the development of intestinal fibrosis in CD patients. Surgical resection is the last resort for severe cases. Thus, new diagnostic and therapeutic approaches to IBD-related intestinal fibrosis are needed.

Antimicrobial peptides such as cathelicidin and lactoferrin have demonstrated clinical utilities as IBD biomarkers. Cathelicidin has anti-inflammatory and anti-fibrogenic effects in colitis models. Elafin is a small (6 kDa) antimicrobial peptide primarily expressed in immune cells, intestinal tract, vagina, lungs, and skin. The potential of elafin as a biomarker has been reported, as elafin blood levels are positively correlated with graft vs. host disease. Increased serum elafin levels were observed in other autoimmune diseases such as rheumatoid arthritis and psoriasis. A microarray study also demonstrated that colonic elafin mRNA expression was increased in ulcerative colitis patients.

Elafin has therapeutic properties in gastrointestinal diseases. Oral administration of elafin-expressing Lactococcus inhibited dextran sulfate sodium (DSS)- and 2,4,6-trinitrobenzene sulfonic acid (TNBS)-mediated colitis in mice and gluten-related disorders in humans. However, the therapeutic potential of elafin in intestinal fibrosis is unknown.

Serum IBD biomarkers are often more convenient than fecal IBD biomarkers in clinical practice. There is no commercially available serum biomarker available for indicating intestinal stricture in Crohn's disease (CD) patients. The IBD biomarker C-reactive protein (CRP) is not a biomarker for CD-associated intestinal stricture formation. Serum levels of LL-37 can be used as a biomarker for CD-associated intestinal strictures. CRP is not a good marker for indicating CD clinical disease activity. LL-37 cannot indicate CD clinical remission. There remains a need for a therapeutic approach to prevent or treat intestinal strictures in CD patients.

### SUMMARY

Described herein is a method of measuring serum elafin levels, elevated levels of which have been confirmed to detect the presence of stricture in patients with Crohn's disease (CD), as diagnosed by endoscopy or imaging. CD patients with high serum elafin levels are at high risk of intestinal strictures. The elafin biomarker test described herein provides a significant savings of time and effort by providing a quick and simple screening tool for gastrointestinal disease. As disclosed herein, serum elafin is better than CRP or LL-37 in indicating intestinal stricture in CD patients. In one embodiment, the method comprises assaying a serum sample obtained from the subject for elafin. The method can be used to detect stricture, for example, when the assay detects an elevated level of elafin relative to a control sample. In some embodiments, the detection of 8,000 pg/ml or more of elafin in the serum sample is indicative of stricture or intestinal fibrosis. In some embodiments, the assay is an immunoassay, such as an enzyme linked immunosorbent assay (ELISA), or a PCR assay, such as real time reverse transcriptase PCR (RT-PCR). Accordingly, also provided is a method of detecting intestinal strictures or a predisposition to intestinal strictures. The method comprises assaying a sample obtained from a subject for elafin, whereby an elevated level of elafin relative to a control sample is indicative of intestinal strictures or a predisposition to intestinal strictures.

Also described herein is a method for improving the accuracy of detecting the presence of stricture through the use of an algorithm developed through machine learning and/or through the use of clinical data. In some embodiments, the method of detecting stricture further comprises determining a probability score, for example, between 0 and 1. In some embodiments, the probability score is based on a serum elafin level in pg/ml and at least three clinical scores. In some embodiments, the clinical scores are selected from the group consisting of: (1) age of the subject in years, (2) years of disease duration, (3) serum C-reactive protein (CRP) level in mg/L, (4) erythrocyte sedimentation rate (ESR) in mm/hour, (5) Harvey Bradshaw Index number (HBI), (6) number of inflammatory bowel disease related surgeries, (7) gender, (8) smoking status, (9) status of biologics (e.g., anti-TNF inhibitor) use, (10) status of steroid use, (11) status of immunomodulator use, (12) status of aminosalicylate use, and (13) presence of fistula. Additional, optional clinical scores include, but are not limited to: (14) serum LL-37 level in ng/ml, (15) serum TGF-b1 level in pg/ml, and (16) serum Cyr61 level in pg/ml. In some embodiments, the probability score is determined using a machine learning algorithm. In some embodiments, a probability score between 0 and 0.5 is indicative of absence of stricture, and a probability score of 0.51 to 1.0 is indicative of stricture. In some embodiments, the algorithm is that available through Microsoft Azure Machine Learning Studio at gallery.cortanaintelligence.com/Experiment/Use-elafin-and-clinical-data-for-indicating-stricture-Predictive-Exp.

Although elafin ameliorates colitis, no report has suggested the therapeutic potential of elafin against intestinal stricture formation in CD patients. Also described herein is the intestinal delivery of an elafin-overexpressing vector to inhibit intestinal fibrosis. The disclosure herein thus describes the first use of elafin as an IBD biomarker and as a therapeutic treatment for CD-associated intestinal strictures.

In one embodiment, the disclosure provides a method of treating intestinal stricture in a subject having Crohn's disease. In a typical embodiment, the method comprises administering elafin to the subject. In one embodiment, a serum sample obtained from the subject has been assayed for elafin, and the assay detects an elevated level of elafin relative to a control sample.

Also provided is a method of inhibiting intestinal fibrosis, inflammatory bowel disease (IBD), metabolic disease, or obesity in a subject, the method comprising administering elafin to the subject. In some embodiments, the elafin is administered to the subject via an elafin-overexpressing vector. In some embodiments, the vector is a bacterial or viral vector. In one embodiment, the vector is a lactic acid bacterium. In some embodiments, the elafin is administered intracolonically. In some embodiments, the elafin is administered orally. In some embodiments, the elafin is administered via a slow release capsule. In some embodiments, the elafin is administered subcutaneously.

### BRIEF DESCRIPTION OF THE DRAWINGS

FIG. 1 Circulating elafin levels are increased in IBD patients. (A) Serum elafin levels of 50 normal, 23 UC, and 28 CD patients in cohort 1 and 20 normal, 57 UC, and 67 CD in cohort 2. Multiple group comparisons were done by one-way ANOVA. (B) Scatter plot shows the moderate correlation between serum elafin levels and clinical disease activity (HBI) in 68 CD patients. (C) Scatter plot shows no association between serum elafin levels and endoscopic disease activity (SES-CD) in 68 CD patients. (D) The stricturing CD patients (n=20) had significantly higher serum elafin levels than non-stricturing CD patients (n=45) in a combined dataset. Two-group comparison was done by Student's t-test. (E) Prevalence, sensitivity, specificity, positive predictive value, negative predictive value, and relative risk of elafin test for indicating intestinal stricture in CD patients.

FIG. 2. A combination of serum elafin levels and clinical data indicates the presence of stricture accurately. (A) ROC curves and AUC values show the accuracy of using elafin data alone for intestinal stricture identification among 67 CD patients (cohorts 1 and 2). The analysis was performed by easyROC web-tool. Cutoff elafin level is 8000 pg/ml. (B) The flowchart of Microsoft Azure machine learning algorithms for indicating intestinal strictures in CD patients. (C) ROC curves and AUC values show the accuracy of using clinical data with or without elafin data for intestinal stricture identification among CD patients. (D) Scatter plot shows the positive correlation between clinical disease activity (Partial Mayo Score) and serum elafin levels in 84 UC patients (cohorts 1 and 2). (E) Scatter plot shows no correlation between serum elafin levels and endoscopic disease activity in 36 UC patients (cohorts 1 and 2).

FIG. 3. Colonic elafin mRNA and protein expression are reduced in stricturing CD patients. (A) Colonic elafin mRNA expression in 40 non-IBD control, 52 UC, and 52 CD patients (Cedars-Sinai cohort). (B) Colonic elafin protein expression in IBD patients. Multiple group comparisons between control, UC, and CD patients were done by one-way ANOVA. (C-D) Colonic elafin mRNA and protein expression in 15 stricturing CD and 28 non-stricturing CD patients. Two-group comparison between CD with stricture and CD without stricture was done by Student's t-test. (E) Immunohistochemistry of elafin in human colonic tissues. Arrows show the elafin protein in mucosal epithelial layers and lamina propria in UC patients. Six patients per group.

FIG. 4. Colonic elafin mRNA and protein expression are negatively correlated with colonic fibrogenic gene mRNA expression in CD patients. (A-B) Percentage of intestinal stricture in CD patients assorted by colonic elafin mRNA and protein expression. Low elafin expression groups had a higher percentage of strictures than high elafin expression groups. (C) Scatter plots show the negative correlations between colonic elafin mRNA and colonic collagen (COL1A2), vimentin (VIM), and TGF-b1 mRNA expression in 20 CD patients (Cedars-Sinai cohort). (D) Scatter plots show the negative correlations between colonic elafin protein expression and colonic collagen (COL1A2), vimentin (VIM), and TGF-b1 mRNA expression in 20 CD patients.

FIG. 5. Mesenteric fat in stricturing CD patients expresses elafin. (A) Mesenteric fat elafin mRNA expression in 36 non-IBD, 31 UC, 37 CD, 11 non-stricturing CD, and 11 stricturing CD patients (Cedars-Sinai cohort). Multiple comparisons between control, UC, and CD patients were done by one-way ANOVA. Two-group comparison between CD with stricture and CD without stricture was done by Student's t-test. (B) Immunohistochemistry of elafin in human mesenteric fat tissues at 200× and 400× magnifications. Elafin (as shown by brown color) protein expression was strong in mesenteric fat adipocytes in stricturing CD patients. Four patients per group. (C) Scatter plot shows the positive correlation between mesenteric fat elafin mRNA expression and colonic fibrogenic gene mRNA expression in 32 CD patients. (D) Scatter plot shows the negative correlation between mesenteric fat elafin mRNA expression and colonic elafin protein expression in 30 CD patients.

FIG. 6. Elafin promotes fibrogenesis in human colonic fibroblasts. (A) The human colonic CCD-18Co fibroblasts were incubated with 100 μl/ml (10%) of human sera from normal, stricturing CD, and non-stricturing CD patients in serum-free DMEM for 24 hours. (B) The human colonic CCD-18Co fibroblasts were incubated with 100 μl/ml (10%) of human sera from high elafin CD group (>8000 pg/ml) and low elafin CD group (<8000 pg/ml) in serum-free DMEM for 24 hours. (C) Serum-starved CCD-18Co fibroblasts were pretreated with 15 μg/ml of anti-elafin neutralizing antibody or control antibody for 30 minutes, followed by exposure to (100 μl/ml) human sera from normal and stricturing CD patients for 24 hours. Six serum donors per group. (D) Serum-starved CCD-18Co fibroblasts were exposed to elafin (0.1-3 μg/ml) for 24 hours. Pro-COL1A1 protein levels in cell lysates were determined by ELISA. (E) Serum-starved primary human intestinal fibroblasts from CD patients were exposed to elafin for 24 hours. mRNA expression was determined by real-time RT-PCR.

FIG. 7. Baseline characteristics of serum samples.

FIG. 8. (A-F) Disease locations (A-B), medications (C), age (D), BMI (E), and duration of disease of IBD patients (F) (Serum sample cohorts).

FIG. 9. Baseline characteristics of colonic and mesenteric fat samples (A). Colonic elafin mRNA expression in UC (B) and CD (C) patients.

FIG. 10. Circulating elafin is moderately accurate in indicating clinical disease activity in CD patients. (A) The stricturing CD patients had higher serum elafin levels than non-stricturing CD patients in two separate datasets from cohorts 1 and 2. The differences were statistically insignificant. Two-group comparison was done by Student's t-test. (B) Serum elafin levels in 18 CD patients with intestinal fistulas versus 67 CD patients without intestinal fistulas in a combined dataset. The difference was statistically insignificant. (C-D) Prevalence, sensitivity, specificity, positive predictive value, negative predictive value, and odds ratio values of elafin test in indicating (C) CD clinical remission and (D) moderate or severe CD clinical activity. (E) ROC curve with AUC value demonstrates the moderate accuracy of using the elafin test for indicating CD clinical disease activity. Optimal cutoff point is 8000 pg/ml.

FIG. 11. Circulating elafin is moderately accurate in indicating clinical disease activity in UC patients. (A-B) Prevalence, sensitivity, specificity, positive predictive value, negative predictive value, and odds ratio values of elafin test in indicating (A) UC clinical remission and (B) moderate or severe UC clinical activity. (C) ROC curve with AUC value demonstrates the moderate accuracy of using the elafin test for indicating UC clinical disease activity. Optimal cutoff point is 18000 pg/ml.

FIG. 12. Colonic elafin mRNA expression is negatively correlated with colonic injury in CD and UC patients. (A-B) Scatter plots show no significant correlation between clinical disease activity and colonic elafin mRNA expression in UC and CD patients. (C-D) Scatter plots show no significant correlation between clinical disease activity and colonic elafin protein expression in 30 UC and 27 CD patients. Simple Clinical Colitis Activity Score for UC patients. Harvey Bradshaw Index for CD patients. (E-F) Scatter plots show the weak negative correlation between colonic histology score and colonic elafin mRNA expression in 30 UC and 27 CD patients. The analysis included 26 UC patients and 29 CD patients.

FIG. 13. Colonic gene signatures of stricturing CD and non-stricturing CD patients are different. (A) Colonic COL1A2 and elafin mRNA expression were determined by real-time RT-PCR and four samples were selected for RNA sequencing. The colonic tissues from stricturing CD patients had high collagen and low elafin mRNA expression. (B) Heat-map of increased (green) and decreased (red) gene expression in the colonic tissues of 2 stricturing CD patients versus 2 non-stricturing CD patients. The RNA-Seq was performed by Omega Biosciences. (C) A list of overexpressed and (D) underexpressed genes in the colonic tissues of CDS patients, compared to CDNS patients. 2 CD patients (HBI =2) per group. >20-fold increased and >9-fold decreased genes in log2(fold change) were shown.

FIG. 14. Serum exosomes from stricturing CD patients stimulate elafin secretion in mesenteric fat adipocytes from CD patients. (A) Serum-starved primary human mesenteric fat adipocytes were exposed to 100 μg/ml serum exosomes from normal, stricturing CD (CDS), or non-stricturing CD (CDNS) patients for 16 hours, followed by incubation with serum-free DMEM media for 6 hours. (B) Serum-starved primary human mesenteric fat adipocytes were exposed to 100 μg/ml serum exosomes from normal or UC patients for 16 hours, followed by incubation with serum-free DMEM media for 6 hours. Conditioned media were collected from elafin ELISA. Each adipocyte group consisted of 5 patients. (C) PBMCs were exposed to 100 μg/ml serum exosomes normal, stricturing CD, non-stricturing CD, and UC patients for 24 hours. (D-E) The human intestinal fibroblasts were incubated with 100 μg/ml of human serum exosomes in serum-free DMEM for 24 hours. The collagen (COL1A2) mRNA expression was determined by real-time RT-PCR. Each serum exosome treatment group consisted of 6 patients per group. Multiple group comparison was done by one-way ANOVA. (E) The human colonic CCD-18Co fibroblasts were incubated with 100 μg/ml of human serum exosomes from high elafin CD group (>8000 μg/ml) and low elafin CD group (<8000 μg/ml) in serum-free DMEM for 24 hours. Each serum exosome treatment group consisted of 6 patients per group. (F) Serum exosomal miRNA expression was determined by real-time RT-PCR. (G) Serum-starved CCD-18Co fibroblasts were treated with miR205-5p power inhibitor for 24 hours. Collagen (COL1A2) and ACTA2 mRNA expression were determined by real-time RT-PCR.

FIG. 15. Elafin directly inhibited collagen expression in intestinal fibroblasts. (A-B) Human colonic CCD-18Co fibroblasts were treated TGF-β1 (10 ng/ml). Two hours later, elafin was added and further incubated for 24 hours. (C) Primary non-IBD patient-derived colonic fibroblasts were pretreated with TGF-β1 (10 ng/ml). Two hours later, elafin (1 μg/ml) was added and further incubated for 24 hours. (D) Primary stricturing CD patient-derived colonic fibroblasts were pretreated with or without 100 μg/ml CDSE or 10 ng/ml TGF-β1. Two hours later, elafin was added and further incubated for 24 hours. COL1A2, ACTA2, and TGF-b1 mRNA expression were determined by real-time RT-PCR. The COL1A2 and ProCOL1A1 protein expression in cell lysates were measured by ELISA. Results were pooled from three independent experiments.

FIG. 16. Systemic elafin overexpression inhibited ileal fibrosis in SAMP1/YitFc mice. (A) Experimental plan. Lentiviral particles were injected into fibrotic 40-week-old SAMP1/YitFc mice intraperitoneally. Non-fibrotic 10-week-old SAMP1/YitFc mice and normal control 42-week-old AKR strain mice were used for comparison. (B) H&E and MT staining. The blue color in MT staining indicated collagen deposition in the lamina propria. (C) Histology score. (D) Fibrosis score. (E) Ileal mRNA expression was determined by real-time RT-PCR.

FIG. 17. Increased ZEB1 expression is associated with intestinal stricture of CD patients. (A) Left: Immunohistochemistry of ZEB1 in colonic tissues of non-IBD, UC, non-stricturing CD, and stricturing CD patients. Right: Colonic ZEB1 mRNA expression in 40 non-IBD, 52 UC, 28 non-stricturing CD, and 15 stricturing CD patients. (B) Positive correlations between colonic ZEB1, collagen (COL1A2), fibroblasts (ACTA2 and vimentin) mRNA expression in CD patients. (C and E) CCD-18Co or CD-HIF fibroblasts were treated TGF-β1 (10 ng/ml) or CDSE (100 μg/ml). Two hours later, elafin was added and further incubated for 24 hours. ZEB1 mRNA/protein and miR205-5p expression were determined by real-time RT-PCR/ELISA. (D) CCD-18Co fibroblasts were transfected with either control-LV or ZEB1-LV for forty-eight hours. The fibroblasts were then treated with TGF-β1 (10 ng/ml). Two hours later, elafin (1 μg/ml) was added and further incubated for 24 hours. miR205-5p and COL1A2 mRNA and ZEB1/ProCOLIA1 protein expression were determined by real-time RT-PCR and ELISA, respectively. (F) CCD-18Co fibroblasts were pretreated with either control inhibitor or miR205-5p inhibitor overnight, followed by TGF-β1 (10 ng/ml) addition. Two hours later, elafin (1 μg/ml) was added and further incubated for 24 hours. COL1A2 mRNA expression was determined by real-time RT-PCR. Results were pooled from three independent experiments.

FIG. 18. miR205-5p inhibitor reversed the anti-fibrogenic effect of intracolonic elafin overexpression in TNBS-treated mice. (A) Experimental plan. Control construct, elafin-overexpressing construct, control inhibitor, and miR205-5p inhibitor were administered to mice intracolonically. Anti-TNFa neutralizing antibodies and miR205-5p-overexpressing lentivirus were injected intraperitoneally. (B) H&E and MT staining. The blue color in MT staining indicated collagen deposition. (C-D) Histology and fibrosis scores. (E) Colonic mRNA expression. Six mice per group.

FIG. 19. ZEB1 overexpression reversed the anti-fibrogenic effect of intracolonic elafin overexpression in TNBS-treated mice. (A) Immunohistochemistry of ZEB1 of colonic tissues of TNBS-treated mice. ZEB1-positive signals are located at lamina propria regions. (B) Experimental plan. Zeb1-overexpressing and Zeb1-shRNA lentivirus were injected into TNBS-treated mice intraperitoneally. Control or elafin-overexpressing construct was injected intracolonically. (C) H&E and MT staining. The blue color in MT staining indicated collagen deposition. (D-E) Histology and fibrosis scores. (F) Colonic mRNA expression. Six mice per group.

FIG. 20. PAR2 agonist reversed the anti-fibrogenic effects of intracolonic elafin overexpression in TNBS-treated mice. (A) Experimental plan. PAR1 and PAR2 agonists were injected intracolonically. GB88 was given via oral gavage. The antibiotic mixture was provided in drinking water ad libitum for the last five days. (B) H&E and MT staining. The blue color in MT staining indicated collagen deposition. (C-D) Histology and fibrosis scores. (E) Colonic mRNA expression. Six mice per group.

FIG. 21. Oral elafin-Eudragit formulation inhibited colonic fibrosis in TNBS-treated mice. (A) Upper panel: Experimental Plan. The elafin-Eudragit formulation was administered to TNBS-treated mice via oral gavage daily. Lower panel: Elafin-Eudragit (10 mg/kg) was administered to normal mice via oral gavage. Colonic elafin levels were determined by ELISA (DY1747, R&D Systems). (B) H&E and MT staining. The blue color in MT staining indicated collagen deposition. (C-D) Histology and fibrosis scores. (E) Colonic mRNA expression. Six mice per group. (F) The anti-fibrogenic pathway of elafin.

FIG. 22. Systemic elafin overexpression inhibited cecal fibrosis in Salmonella-infected mice. (A) Experimental plan. Control or elafin-overexpressing lentivirus were injected intraperitoneally. Antibiotic mixture was provided in drinking water ad libitum. (B) H&E and MT staining. The blue color in MT staining indicated the deposition of collagen in the cecal lamina propria of Salmonella-infected mice. (C-D) Histology and fibrosis scores. (E) Cecal collagen and fibrogenic mediator (Col1a2 and Zeb1), fibroblast marker (Acta2 and Vim), and inflammatory markers (Tnf and Emr1) mRNA expression. Note: Salmonella infection did not increase cecal Col3a1 mRNA expression in mice. Lentiviral elafin expression reversed cecal fibrosis in the Salmonella-infected mice, which was not affected by antibiotics. Five mice per group.

FIG. 23. The anti-fibrogenic effects of elafin in Salmonella-infected mice was miR205-5p-dependent. (A) Experimental plan. Control or elafin-overexpressing lentivirus, miR205-5p-overexpressing (OE) lentivirus, or miR205-5p inhibitory (OFF) lentivirus were injected intraperitoneally. (B) H&E and MT staining. (C-D) Histology and fibrosis scores. (E) Cecal collagen and fibrogenic mediator (Col1a2 and Zeb1), fibroblast markers (Acta2 and Vim), and inflammatory markers (Tnf and Emrl) mRNA expression. Lentiviral miR205-5p overexpression (OE) inhibited cecal fibrosis in Salmonella-infected mice. Lentiviral elafin overexpression reversed cecal fibrosis, which was reversed by lentiviral miR205-5p inhibition (OFF). Five mice per group.

FIG. 24. The anti-fibrogenic effects of elafin in Salmonella-infected mice was Zeb1-dependent. (A) Experimental plan. Control or elafin-overexpressing lentivirus, Zeb1-overexpressing lentivirus, or Zeb1-shRNA inhibitory lentivirus were injected intraperitoneally. (B) H&E and MT staining. (C-D) Histology and fibrosis scores. (E) Cecal collagen and fibrogenic mediator (Col1a2 and Zeb1), fibroblast markers (Acta2 and Vim), and inflammatory markers (Tnf and Emr1) mRNA expression. Lentiviral Zeb1shRNA inhibition reduced cecal fibrosis in Salmonella-infected mice. Lentiviral elafin overexpression inhibited cecal fibrosis, which was reversed by lentiviral Zeb1overexpression. Five mice per group.

FIG. 25. The anti-fibrogenic effects of elafin in Salmonella-infected mice was PAR2-dependent. (A) Experimental plan. Control or elafin-overexpressing lentivirus were injected intraperitoneally. PAR2 inhibitor GB88 and PAR2 agonist GB110 were given via oral gavage. (B) H&E and MT staining. (C) Histology score. (D) Fibrosis score. (E) Cecal collagen and fibrogenic mediator (Col1a2 and Zeb1), fibroblast markers (Acta2 and Vim), and inflammatory markers (Tnf and Emrl) mRNA expression. GB88 inhibited cecal fibrosis. Lentiviral elafin overexpression inhibited cecal fibrosis, which was reversed by GB110. Five mice per group.

FIG. 26. Ileal microbiome data of elafin-overexpressing SAMP1/YitFc mice. (A-C) 16S sequencing analysis of mouse ileal microbiome. (A) Alpha diversity (bacterial species diversity within a sample) in ileal microbiota samples was represented by the Shannon index. (B) Beta diversity of the ileal microbiota samples (how samples are different from each other) is shown in the principal coordinate analysis (PCoA) plot. (C) Ileal microbiome composition at the genus level in each sample group. Family is shown for microbes that could not be assigned to the genus levels. Four mice per group. (D) Antibiotics-treated SAMP1/YitFc mice with and without lentiviral elafin overexpression. H&E and MT staining. (E) Histology and fibrosis scores. (F) Colonic mRNA expression. Four mice per group.

FIG. 27. Elafin inhibited ERK1/2 phosphorylation in intestinal fibroblasts via PAR2. (A) Serum starved CCD-18Co and primary stricturing CD patient-derived colonic fibroblasts were transiently transfected with either control siRNA or ZEB1 siRNA overnight, followed by treatment with 10 ng/ml TGF-β1 or 100 μg/ml stricturing CD patient-derived serum exosomes (CDSE) for 24 hours. (B) CCD-18Co fibroblasts were treated with DMSO, PAR1 inhibitor (SCH79797), or PAR2 inhibitor (GB88) for 2 hours. GB88 inhibited ERK1/2 phosphorylation. (C) CCD-18Co fibroblasts were pretreated with DMSO, PAR1 agonist, or PAR2 agonists for 30 minutes, followed by TGF-β1 10 ng/ml. Two hours later, elafin (1 μg/ml) and incubated for 2 hours. Elafin inhibited ERK1/2 phosphorylation, which was reversed by PAR2 agonist. (D) CCD-18Co or primary stricturing CD patient-derived colonic fibroblasts were pretreated with DMSO or GB88 for 30 minutes, followed by incubation with 10 ng/ml TGF-β1 or 100 μg/ml stricturing CD patient-derived serum exosomes (CDSE) for 24 hours. (E-F) Serum-starved CCD-18Co fibroblasts were pretreated with DMSO, PAR1 agonist (10 μM), or PAR2 agonist (10 μM). An hour later, TGF-β1 (10 ng/ml) was added. Two hours later, elafin (1 μg/ml) added and further incubated for 24 hours. Phospho-ERK1/2, ProCOL1A1, COL1A2, and ZEB1 protein were determined by ELISA. miR205-5p expression was determined by real-time RT-PCR. Results were pooled from three independent experiments.

FIG. 28. Elafin reduced fibrogenesis via inhibition of cathepsin S-mediated PAR2 activity in fibroblasts. (A-B) Serum starved CCD-18Co or primary stricturing CD patient-derived colonic fibroblasts were pretreated with DMSO, PAR1 agonist (10 μM), or PAR2 agonist (10 μM). An hour later, the fibroblasts were exposed to 10 ng/ml TGF-β1 or 100 μg/ml stricturing CD patient-derived serum exosomes (CDSE). Two hour later, elafin (1 μg/ml) added and further incubated for 24 hours. (C) Serum starved primary stricturing CD patient-derived colonic fibroblasts were treated with 100 □g/ml stricturing CD patient-derived serum exosomes (CDSE) for 2 hours. Serum starved CCD-18Co colonic fibroblasts were treated with TGF-β1 (10 ng/ml) for 2 hours. Conditioned media were loaded to Proteome Profiler Human Protease Arrays (ARY021B, R&D Systems). The images were captured by Bio-Rad ChemiDoc Imaging system. The rectangles highlighted increased Cathepsin S expression after exposure to CDSE or TGF-β1. (D) Quantification of cathepsin S signals (B7-8) and control signals (A1-2 and E1-2) on the protease array using Bio-Rad Image Lab Software. (E) CCD-18Co fibroblasts were pretreated with either TFA 0.1% or cathepsin S (0.4 μg/ml) for 30 minutes, followed by incubation with elafin 1 μg/ml for 2 hours. Phospho-ERK1/2 was determined by ELISA. (F) Serum starved CCD-18Co and primary stricturing CD patient-derived colonic fibroblasts were pretreated with either TFA 0.1% or cathepsin S (0.4 □g/ml) for 30 minutes, followed by treatment with TGF-β1 (10 ng/ml) or 100 μg/ml stricturing CD patient-derived serum exosomes (CDSE). Two hours later, elafin 1 μg/ml was added and further incubated for 24 hours. ProCOL1A1 and COL1A2 were determined by ELISA. Results were pooled from three independent experiments.

FIG. 29. Elafin increased intestinal miR205-5p expression in fibrotic mice. (A) Primary non-IBD colonic fibroblasts were transfected with either control mimic or miR205-5p mimic overnight, followed by incubation with either 0.1% TFA or TGF-β1 (10 ng/ml) for 24 hours. miR205-5p mimic inhibited TGF-1β induced ProCOL1A1 protein expression. (B) Ileal miR205-5p expression in 42-week-old SAMP1/YitFc mice. (C) Cecal miR205-5p expression in Salmonella-infected mice (day 21). (D-E) Colonic miR205-5p expression in TNBS-treated mice (7th week). (F) Colonic miR205-5p expression in the colons of 40 non-IBD, 52 UC, 28 non-stricturing CD, and 15 stricturing CD patients.

FIG. 30. Cytokine levels in mouse and human sera and CD-PBMCs. (A) Mouse serum cytokine levels were measured by a 23-plex multiplex assay (#M60009RDPD, Bio-Rad). n=5 mice per group. (B) Human serum cytokines were measured by a 27-plex multiplex assay (#M500KCAFOY, Bio-rad). 12 normal, 23 UC, and 43 CD patients. (C) CD patient-derived PBMCs were preconditioned with stricturing CD patient (CDS) or non-stricturing CD patient (CDNS)-derived serum exosomes for two hours, followed by exposure to either 0.1% TFA or 1 μg/ml elafin for 6 hours. The cell supernatants were collected for a 13-plex cytokine multiplex assay (HSTCMAG28SPMX13, Millipore Sigma). (D) A comparison of cytokine levels in mouse sera, human sera, and conditioned media of CDS and CDNS serum exosome-conditioned CD-PBMCs. ns=no significant difference. ND=below detection range. N/A=unavailable. Mo=monocytes. MO=macrophages, DC=dendritic cells.

FIG. 31 Serum elafin levels were inversely correlated with hyperglycemia and hyperinsulinemia in men with T2DM. (A, B) Serum elafin and fasting blood glucose levels in patients. (C) The inverse correlation between serum elafin levels and fasting blood glucose levels in men with T2DM. No correlation between elafin and fasting blood glucose levels in patients without prediabetes/diabetes, patients with prediabetes, and women with T2DM. (D) HbA1c levels in patients. (E) The inverse correlation between serum elafin levels and HbA1c levels in men with T2DM but not women with T2DM. (F) Fasting blood insulin levels in patients. (G) The inverse correlation between serum elafin levels and fasting blood insulin levels in men with T2DM but not women with T2DM. (H) There is no association between serum elafin levels and age among all patients.

FIG. 32. Lentiviral elafin overexpression reduced food consumption and fat mass in HFD-treated male mice. (A, upper panel) RD/HFD/HCD treatment and lentiviral elafin overexpression in mice. (A, lower panel) Ct values in the real-time RT-PCR experiments for detecting the presence of elafin mRNA signal in the adipose tissues of HFD-treated mice. Each group consists of 6 mice. (B) Change in body weight over 14 days. (C) Change in percentage of fat mass. (D) Fasting blood glucose levels. (E) Daily food consumption. (F) Serum leptin levels. Elafin did not affect food consumption, fat mass, or fasting blood glucose levels in ob/ob mice and HCD-treated mice. (G) Serum leptin levels in patients. Women have significantly higher serum leptin levels than men in all groups. (H) The correlation between serum elafin levels and fasting blood glucose levels in patients. Serum elafin levels are positively correlated with leptin levels in men with T2DM.

FIG. 33. Circulating immune cells mediated the anti-obesity effects of elafin in HFD-treated male mice. (A) Circulating immune cell mRNA expression in the HFD-treated mice with and without lentiviral elafin overexpression. (B) Splenocyte transplantation to HFD-treated Rag−/− mice. (C) Physiological parameters of HFD-treated Rag−/− recipient mice after splenocyte transplantation. Transplantation of splenocytes from elafin-overexpressing donors caused reduced fat mass, body weight, and food consumption and increased leptin levels in the Rag−/− recipient mice. (D) Mesenteric fat and epididymal fat tissue mRNA expression in HFD- treated mice with and without lentiviral elafin overexpression. (E) Determination of the physiological effects of intraperitoneal IL-1β and IFNγ injection to HFD-treated elafin-expressing mice. IL-1β or IFNγ was injected on the same day as elafin-expressing lentivirus injection. (F) Daily food consumption, (G) serum leptin levels. (H) Change in body weight. (I) Change in fat mass. (J) Fasting blood glucose levels. Each group consists of 8 mice.

FIG. 34. Immune cell-derived miR181b-5p and miR219-5p induced leptin mRNA expression in 3T3-L1 adipocytes. (A) Mouse serum exosomal miRNA expression was profiled by a PCR array. miR219-5p, miR210-3p, and miR181-5p were undetectable in the serum exosomes of HFD-treated mice without elafin overexpression. These three miRNAs were detected in the serum exosomes of elafin-overexpressing HFD-treated mice only. Each group consists of 6 mice. (B) miR219-5p, miR210-3p, and miR181-5p expression in the circulating immune cells were significantly increased in the elafin-overexpressing HFD-treated mice, compared to HFD- treated mice without elafin overexpression. (C) The positive correlation between serum elafin levels and miRNAs (miR181b-5p and miR210-3p, but not miR219-5p) in the men with T2DM. (D) The negative correlation between fasting blood glucose levels and miRNAs (miR181b-5p and miR210-3p, but not miR219-5p) in men with T2DM. (E) The positive correlation between serum leptin levels and miRNAs (miR181b-5p and miR210-3p, but not miR219-5p) in the men with T2DM. (F) Oil red 0 staining of serum-starved 3T3-L1 adipocytes after 24-h exposure to elafin (10 ng/ml, no transfection), miRNA mimics (75 ng/ml via overnight transfection), or mouse serum exosomes (10 μg/ml). (G) Leptin, Cd36, and adiponectin mRNA expression in 3T3-L1 adipocytes after 24-h exposure to elafin (10 ng/ml, no transfection), miRNA mimics (75 ng/ml via overnight transfection), and serum exosomes (10 μg/ml). Results were pooled from three independent experiments.

FIG. 35. Elafin-dependent serum exosome-mediated inhibition of hyperphagia and hyperglycemia was reversed by miR181b-5p and miR219-5p inhibitors. (A) Serum exosome transplantation to HFD-treated mice. The miRNA inhibitors were injected on the same day as exosome injection. (B) Change in fat mass. (C) No significant change in body weight among all groups over 7 days after miRNA inhibitor and exosome injection. (D) Fasting blood glucose levels 7 days after exosome and miRNA inhibitor injection. (E) Daily food consumption per mouse. (F) Serum leptin levels measured on day seven post-injection. (G, H) Leptin, Cd36, and adiponectin mRNA expression in the mesenteric fat and epididymal fat of HFD-treated mice with exosome and miRNA inhibitor injection. (I) Leptin sensitivity test: The RD/HFD-treated mice were injected with leptin for 3 days after elafin-LV injection. (J, K) Daily food consumption and fasting blood glucose levels at 24 h after the last leptin injection. Each group consists of 8 mice.

FIG. 36. Subcutaneous and oral administration of modified elafin inhibited hyperphagia and hyperglycemia in HFD-treated male mice. (A) Elafin (1 mg/kg) was injected into RD-treated male mice subcutaneously. Tail-vein blood samples were collected for elafin ELISA. This mouse experiment was intended for the determination of basic pharmacokinetics of elafin, but not the determination of physiology of elafin. (B) HFD-treated mice were treated with either oral gavage of Elafin-Eudragit formulation (10 mg/kg) daily or subcutaneous injection of PEG-elafin (3.25 mg/kg) every 48 h for 14 days. Serum elafin levels. (C) Serum leptin levels. (D) Serum IFNγ and IL-1β levels. (E) Daily food consumption. (F) Change in fat mass. (G) Change in body weight over 14 days. (H) Fasting blood glucose levels. Each group consists of 8 mice.

### DETAILED DESCRIPTION

Described herein are methods and kits for detecting and treating intestinal stricture, developed through the surprising discovery that circulating elafin levels positively correlate with clinical disease activity and the presence of stricture. Intestinal elafin overexpression provides a novel therapeutic strategy against intestinal fibrosis

Elafin is the best indicator of CD intestinal stricture thus far. Elafin is a surrogate biomarker of IBD disease activity. Low colonic elafin protein expression is associated with intestinal stricture in Crohn's disease patients. Elafin inhibits collagen synthesis in human colonic fibroblasts in a G protein-coupled receptor-independent pathway. Intracolonic administration of elafin overexpressing constructs inhibit colitis-associated intestinal fibrosis in a mouse model of chronic colitis.

Colonic elafin mRNA expression is increased in UC, but not CD, patients. Colonic elafin mRNA expression is inversely correlated to histology score of CD patients. More details are provided in the Examples below. However, Elafin is associated with higher relative risk of intestinal stricture formation than LL-37. Therefore, elafin is more accurate than LL-37 or CRP as an indicator of the presence of intestinal strictures. CRP is not a good marker for indicating CD clinical disease activity. Low elafin levels represent an accurate biomarker indicating CD clinical remission. LL-37 cannot indicate CD clinical remission. Elafin may be expressed in the intestine using elafin-overexpressing bacteria or DNA vector, providing a novel therapeutic or preventive drug target against intestinal strictures in CD.

### Definitions

All scientific and technical terms used in this application have meanings commonly used in the art unless otherwise specified. As used in this application, the following words or phrases have the meanings specified.

As used herein, a “control” or “reference” sample means a sample that is representative of normal measures of the respective marker, such as would be obtained from normal, healthy control subjects, or a baseline amount of marker to be used for comparison. Typically, a baseline will be a measurement taken from the same subject or patient. The sample can be an actual sample used for testing, or a reference level or range, based on known normal measurements of the corresponding marker.

As used herein, a “significant difference” means a difference that can be detected in a manner that is considered reliable by one skilled in the art, such as a statistically significant difference, or a difference that is of sufficient magnitude that, under the circumstances, can be detected with a reasonable level of reliability. In one example, an increase or decrease of 10% relative to a reference sample is a significant difference. In other examples, an increase or decrease of 20%, 30%, 40%, or 50% relative to the reference sample is considered a significant difference. In yet another example, an increase of two-fold relative to a reference sample is considered significant. As described herein, in some embodiments, a cut point is used to identify a significant increase or decrease.

“Nucleotide sequence” refers to a heteropolymer of deoxyribonucleotides, ribonucleotides, or peptide-nucleic acid sequences that may be assembled from smaller fragments, isolated from larger fragments, or chemically synthesized de novo or partially synthesized by combining shorter oligonucleotide linkers, or from a series of oligonucleotides, to provide a sequence which is capable of expressing the encoded protein.

As used herein, “pharmaceutically acceptable carrier” or “excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well-known conventional methods (see, for example, Remington's Pharmaceutical Sciences, 18th edition, A. Gennaro, ed., Mack Publishing Co., Easton, PA, 1990).

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects.

As used herein, a “processor” refers to a device capable of processing information, such as in the form of signal processing. One example of a processor is a digital signal processor circuit or an application specific integrated circuit (ASIC). One or more processors may be contained within a single device.

As used herein, an “analyzer” refers to a device capable of analyzing data. One or more analyzers may be contained within a single device.

As used herein, “a” or “an” means at least one, unless clearly indicated otherwise.List of abbreviations:

AUC=Area Under Curve

CD=Crohn's Disease

CRP=C-reactive Protein

ESR=Erythrocyte Sedimentation Rate

FC=Fecal Calprotectin

HBI=Harvey Bradshaw Index

IBD=Inflammatory Bowel Disease

LL-37=Human Cathelicidin

NPV=Negative Predictive Value

PMS=Partial Mayo Score

PPV=Positive Predictive Value

ROC=Receiver Operating Characteristics

UC=Ulcerative Colitis

### Methods of the Invention

Described herein is a method of treating a subject having intestinal fibrosis and/or intestinal stricture. The method comprises administering to the subject elafin in a pharmaceutically acceptable form. Administration can be direct or indirect, and examples of delivery routes include, but are not limited to, intracolonic, intravenous, or oral. Elafin may be directly delivered to the affected intestine by colonoscopy. In some embodiments, elafin is delivered to the subject by intracolonic administration of elafin-overexpressing vector. In some embodiments, administration is by elafin-containing pH-release capsules that release elafin in ileum and colon. In some embodiments, the elafin is administered subcutaneously.

In one embodiment, the invention provides a method of treating intestinal stricture in a subject having Crohn's disease. In a typical embodiment, the method comprises administering elafin to the subject. In some embodiments, the elafin has the full amino acid sequence (including signal sequence): MRASSFLIVVVFLIAGTLVLEAAVTGVPVK GQDTVKGRVPFNGQDPVKGQVSVKGQDKVKAQEPVKGPVSTKPGSCPIIL IRCAMLNPPNRCLKDTDCPGIKKCCEGSCGMACFVPQ (SEQ ID NO: 1). In some embodiments, the elafin has the amino acid sequence: AQEPVKGPVSTKPGSCPIIL IRCAMLNPPNRCLKDTDCPGIKKCCEGSCGMACFVPQ (SEQ ID NO: 2). The foregoing sequence represents a natural and active form of elafin, which has been shown herein to have therapeutic efficacy. In some embodiments, such as for oral administration, one can package elafin having this sequence in Eudragit. Alternatively, in some embodiments, one can administer a lentivirus or DNA construct that expresses this sequence of elafin. In some embodiments, the amino acid sequence is modified and/or conjugated to other substances for improved anti-fibrogenic or anti-inflammatory effects.

In one embodiment, a serum sample obtained from the subject has been assayed for elafin, and the assay detects an elevated level of elafin relative to a control sample. Treatment is then administered to the subject exhibiting elevated serum elafin. In some embodiments, the elevated elafin is greater than or equal to 8,000 μg/ml.

Also provided is a method of inhibiting or treating intestinal fibrosis, inflammatory bowel disease (IBD), metabolic disease, or obesity in a subject. The method comprises administering elafin to the subject. In some embodiments, the elafin is administered to the subject via an elafin-overexpressing vector. Elafin may be expressed in the intestine using elafin-overexpressing bacteria or DNA vector. Examples of an elafin-overexpressing vector include, but are not limited to, elafin-overexpressing lactic acid bacteria. Other means of administering elafin as a method of treating a subject having intestinal fibrosis and/or stricture include, for example, elafin-containing pH-release capsules or elafin mimics.

In one embodiment, described is a method of detecting intestinal stricture in a subject. The method comprises assaying a serum sample obtained from the subject for elafin. Elafin can be detected in serum samples using immunoassay techniques, such as ELISA. Elafin levels >8000 μg/ml significantly indicates the presence of intestinal stricture in CD patients with relative risk=2.45. Elafin protein can be detected in colonic tissues using immunoassay techniques, such as ELISA, and amplification-based detection means, such as real-time RT-PCR. Elafin expression in colonic tissues exhibits an inverse relationship with occurrence of intestinal stricture in CD patients. CD patients with low colonic elafin protein levels (<140 μg/μg protein) are more likely (odds ratio 15, p=0.0379) than high colonic elafin protein levels to have intestinal stricture.

In some embodiments, the method further comprises use of clinical data and/or a machine learning algorithm. These steps improve the accuracy of detection. Also described herein is a method for improving the accuracy of detecting the presence of stricture through the use of an algorithm developed through machine learning and/or through the use of clinical data. In some embodiments, the method of detecting stricture further comprises determining a probability score, for example, between 0 and 1. In some embodiments, the probability score is based on a serum elafin level in pg/ml and at least three clinical scores.

In some embodiments, the clinical scores are selected from the group consisting of: (1) age of the subject in years, (2) years of disease duration, (3) serum C-reactive protein (CRP) level in mg/L, (4) erythrocyte sedimentation rate (ESR) in mm/hour, (5) Harvey Bradshaw Index number (HBI), (6) number of inflammatory bowel disease related surgeries, (7) gender, (8) smoking status, (9) status of biologics (e.g., anti-TNF inhibitor) use, (10) status of steroid use, (11) status of immunomodulator use, (12) status of aminosalicylate use, and (13) presence of fistula. Additional, optional clinical scores include, but are not limited to: (14) serum LL-37 level in ng/ml, (15) serum TGF-b1 level in pg/ml, and (16) serum Cyr61 level in μg/ml. In some embodiments, the probability score is determined using a machine learning algorithm. In some embodiments, a probability score between 0 and 0.5 is indicative of absence of stricture, and a probability score of 0.51 to 1.0 is indicative of stricture. In some embodiments, the algorithm is that available through Microsoft Azure Machine Learning Studio at gallery.cortanaintelligence.com/Experiment/Use-elafin-and-clinical-data-for-indicating-stricture-Predictive-Exp.

The study described in the Examples herein used the same serum patient cohorts as described in BMC Gastroenterol. 2017 May 12; 17(1):63. doi: 10.1186/s12876-017-0619-4. Circulating cathelicidin levels correlate with mucosal disease activity in ulcerative colitis, risk of intestinal stricture in Crohn's disease, and clinical prognosis in inflammatory bowel disease, and commercially available ELISA for measuring serum elafin levels in IBD patients. Serum Elafin levels are significantly increased in UC and CD patients. Elafin <18000 pg/ml significantly indicates UC clinical remission (PMS=0-1) with odds ratio=9 Elafin <8000 pg/ml significantly indicates CD clinical remission (HBI=0-4) with odds ratio=3.5 Elafin >8000 pg/ml significantly indicates CD moderate or severe activity (HBI=5-9) with odds ratio 7.5 Elafin >8000 pg/ml significantly indicates the presence of intestinal stricture in CD patients with relative risk=2.45, which is higher than LL-37 or CRP. Elafin is the best indicator of CD intestinal stricture so far. Elafin is a surrogate biomarker of IBD disease activity. Low colonic elafin protein expression is associated with intestinal stricture in Crohn's disease patients. Elafin inhibits collagen synthesis in human colonic fibroblasts in a G protein-coupled receptor-independent pathway. Intracolonic administration of elafin overexpressing constructs inhibit colitis-associated intestinal fibrosis in a mouse model of chronic colitis. We included the same colonic tissue cohorts as in Sci Rep. 2017 Nov. 27; 7(1):16351. doi: 10.1038/s41598-017-16753-z. CSA13 inhibits colitis-associated intestinal fibrosis via a formyl peptide receptor like-1 mediated HMG-CoA reductase pathway.

### Kits and Assay Standards

Also provided are kits comprising a set of reagents as described herein, such as antibodies or probes that specifically bind elafin, and optionally, one or more suitable containers containing reagents of the invention. Reagents include molecules that specifically bind elafin, directly or indirectly. Some examples of a reagent include an antibody, primer, or probe that is specific for the marker. Reagents can optionally include a detectable label. Labels can be fluorescent, luminescent, enzymatic, chromogenic, or radioactive.

Kits of the invention optionally comprise an assay standard or a set of assay standards, either separately or together with other reagents. An assay standard can serve as a normal control by providing a reference level of normal expression for a given marker that is representative of a healthy individual.

Kits can include probes for detection of alternative gene expression products in addition to antibodies for protein detection. The kit can optionally include a buffer. Optionally, the kit can include reagents for detecting additional expression products and other markers of interest.

Also provided are compositions comprising a therapeutically effective amount of elafin or a construct that expresses elafin. In some embodiments, the composition is a pharmaceutical composition, optionally further comprising a pharmaceutically acceptable excipient, carrier, or other agents to facilitate effective treatment. In some embodiments, the composition further comprises one or more additional therapeutic agents.

### EXAMPLES

The following examples are presented to illustrate the present invention and to assist one of ordinary skill in making and using the same. The examples are not intended in any way to otherwise limit the scope of the invention.

### Example 1

### High Circulating Elafin Levels are Associated with Crohn'S Disease-Associated Intestinal Strictures

Antimicrobial peptide expression is associated with disease activity in inflammatory bowel disease (IBD) patients. IBD patients have abnormal expression of elafin, a human elastase-specific protease inhibitor and antimicrobial peptide. This example determined elafin expression in blood, intestine, and mesenteric fat of IBD and non-IBD patients. This example demonstrates that high circulating elafin levels are associated with the presence of stricture in CD patients. Serum elafin levels may help identify intestinal strictures in CD patients.

Serum samples from normal and IBD patients were collected from two UCLA cohorts. Surgical resection samples of human colonic and mesenteric fat tissues from IBD and non-IBD (colon cancer) patients were collected from Cedars-Sinai Medical Center.

High serum elafin levels were associated with a significantly elevated risk of intestinal stricture in Crohn's disease (CD) patients. Microsoft Azure Machine learning algorithm using serum elafin levels and clinical data identified stricturing CD patients with high accuracy. Serum elafin levels had weak positive correlations with clinical disease activity (Partial Mayo Score and Harvey Bradshaw Index), but not endoscopic disease activity (Mayo Endoscopic Subscore and Simple Endoscopic Index for CD) in IBD patients. Ulcerative colitis (UC) patients had high serum elafin levels. Colonic elafin mRNA and protein expression were not associated with clinical disease activity and histological injury in IBD patients, but stricturing CD patients had lower colonic elafin expression than non-stricturing CD patients. Mesenteric fat in stricturing CD patients had significantly increased elafin mRNA and protein expression, which may contribute to high circulating elafin levels. Human mesenteric fat adipocytes secrete elafin protein.

Intestinal stricture formation is a debilitating complication of inflammatory bowel disease (IBD) [1]. Chronic inflammation in Crohn's disease (CD) patients leads to multiple cycles of tissue injury and healing [2]. The transforming growth factor-beta 1 (TGF-β1)-activated myofibroblasts produce an excessive amount of extracellular matrix, such as collagen, in the submucosa and mucosa [3], which obstructs bowel movement.

Around one-third of CD patients develop strictures (Vienna classification B2) over ten years after diagnosis [4]. The IBDchip European project that included 1528 CD patients with more than ten years of follow-up showed 48.2% of patients with stricturing behavior [5]. Imaging and endoscopic evaluations of intestinal strictures are expensive and time-consuming [6, 7]. Several serum factors (miR-19, miR29, collagen, fibronectin, tissue inhibitor of matrix metalloproteinase-1, basic fibroblasts growth factor, chitinase 3-like 1 (YKL-40), anti-Saccharomyces cerevisiae antibodies, and fibrocytes) had shown conflicting results with low specificity for stricturing CD patients [8]. We are interested in discovering novel biomarkers for intestinal strictures because there are none established for indicating the presence of intestinal strictures.

Antimicrobial peptides and proteins such as serum cathelicidin, stool lactoferrin, and fecal calprotectin (FC) demonstrated clinical utilities as IBD biomarkers [9, 10]. Fecal calprotectin is useful for assessment of IBD disease activity [11]. Cathelicidin has anti-inflammatory and anti-fibrogenic effects in colitis models [12-15]. Elafin is a small (6 kDa) elastase-specific protease inhibitor with antimicrobial functions, primarily expressed in immune cells, intestinal tract, vagina, lungs, and skin [16]. Increased serum elafin levels are significantly associated with the presence of rheumatoid arthritis and the diseased area of psoriasis [17, 18].

Colonic elafin mRNA expression was increased in ulcerative colitis (UC) patients [19]. However, there was no increase of colonic elafin mRNA and protein expression in CD patients [20]. Zhang's group reported the reduced elafin mRNA expression in peripheral blood leukocytes of IBD patients [21, 22]. However, the relevance of elafin in intestinal strictures is unknown. Interestingly, UVA irradiation induces elafin expression in dermal fibroblasts, leading to the accumulation of elastic fibers in the actinic elastosis of sun-damaged skin [23]. Therefore, this evidence suggests the potential association between elafin and fibrogenesis.

A recent study suggests that mesenteric fat wrapping (creeping fat) may be associated with the risk of intestinal stricture in CD patients, but the mechanism of this association is unknown and has not yet been identified [24]. Elafin expression in the adipose tissue of IBD patients is unknown. We hypothesize that a link between elafin expression and intestinal fibrosis may exist. This study examined the expression of elafin in circulation, intestine, and mesenteric fat in non-IBD, UC, stricturing CD, and non-stricturing CD patients.

Materials and Methods

Human Serum Samples

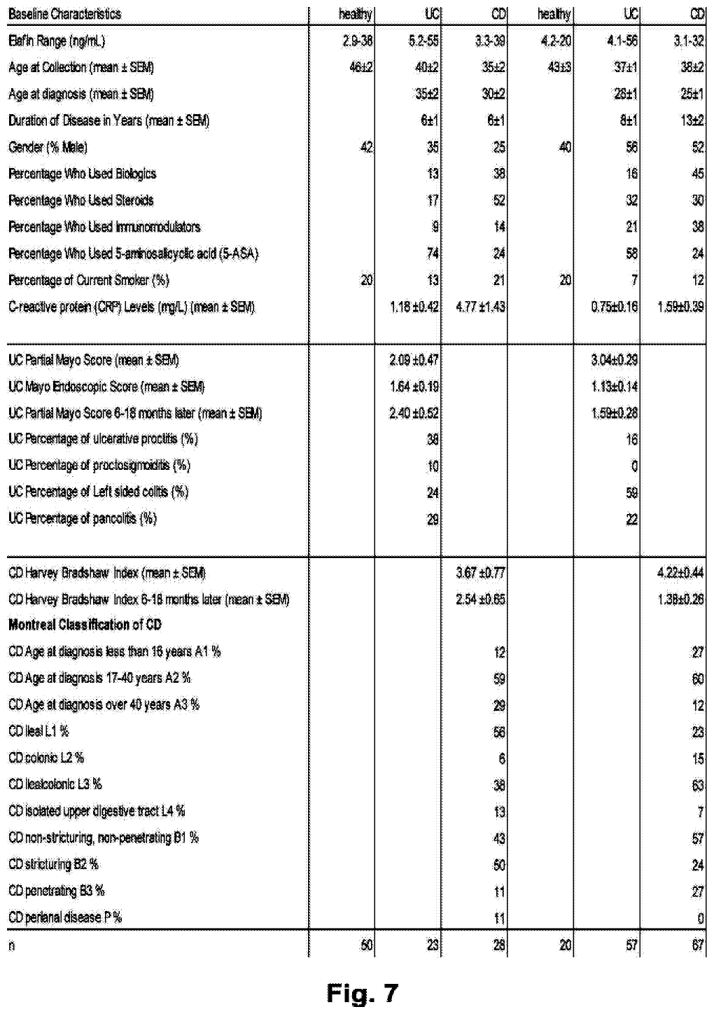

For serum samples, IBD patients of cohort 1 were recruited from UCLA Gastroenterology clinic, and control normal patients of cohort 1 were recruited from UCLA Internal Medicine clinic. This cohort consists of 50 healthy, 23 UC, and 28 CD patients (S1 Table in FIG. 7). All serum samples of cohort 1 were prepared by UCLA Department of Pathology. All serum samples from cohort 2 were obtained from UCLA Center for IBD Biobank, which consists of 20 healthy, 57 UC, and 67 CD patients. Patients of these two cohorts did not overlap. All samples were collected during the indicated diagnostic procedure between 2012-2015 prospectively. The serum sample study was approved by the UCLA Institutional Review Board (protocol number IRB 12-001499 and IRB 13-001069). Written informed consent was obtained from all subjects by either UCLA Pathology or IBD Center. Separate informed consent was waived by UCLA IRB.

Inclusion criteria: IBD diagnosis was confirmed by UCLA gastroenterologists. Both cohorts included patients with a wide range (from remission to severe) of clinical and endoscopic disease activity. Intestinal strictures in CD patients were identified by magnetic resonance enterography (MRE), computed tomography (CT), or endoscopy. The intestinal strictures in CD patients were defined by prestenotic dilation, luminal narrowing, and increased wall thickness. The gastroenterologists requested the IBD patients to blood collection procedures as medically indicated. The internal medicine physician requested the normal patients to blood collection procedures as medically indicated. The healthy subjects (control group) visited the UCLA Internal Medicine clinic for regular body checkups. The healthy subjects did not have concurrent cancer, infection, obesity (BMI>30), prediabetes, or diabetes.

Exclusion criteria: Pregnant women, prisoners, or minors under age 18 were not included. Additionally, IBD patients with concurrent acute infection (CMV, C. difficile, and tuberculosis) and malignant conditions were excluded. Serum samples with hemolysis were excluded.

Human Colonic and Mesenteric Fat Samples

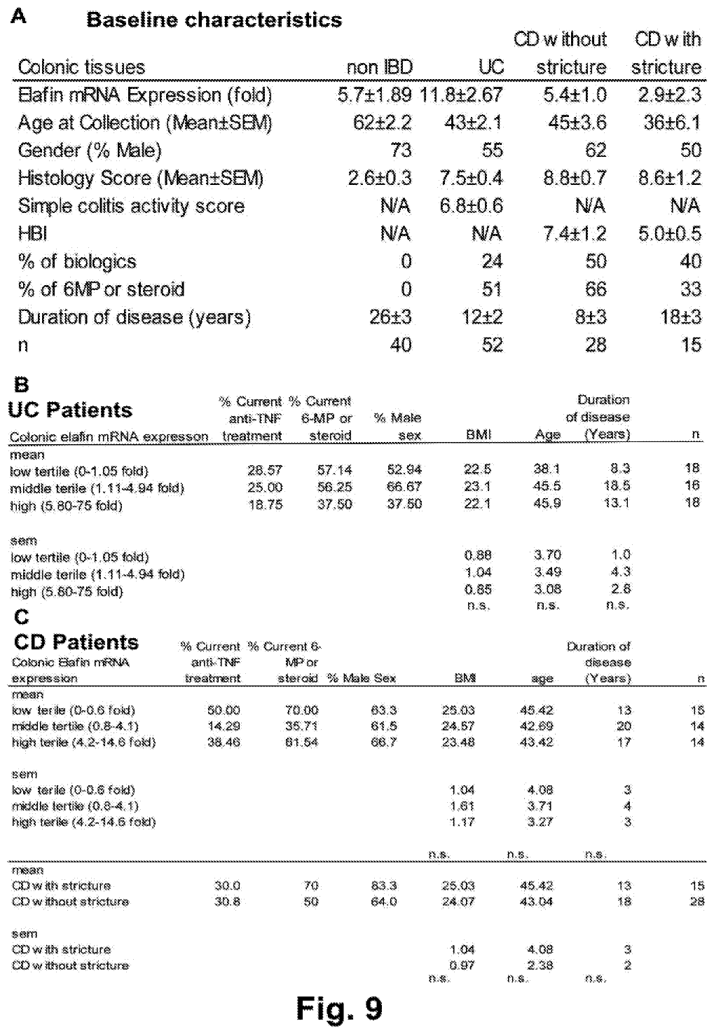

Patient-matched human colonic and mesenteric fat samples were collected from the Cedars-Sinai Medical Center [25]. This cohort consists of 40 non-IBD, 52 UC, 28 non-stricturing CD, and 15 stricturing CD patients (S3 Table in FIG. 9). All colonic and mesenteric fat samples were collected at the same time from surgical operations. All samples were collected during the indicated diagnostic procedure between 2010-2014 prospectively. The colonic and fat sample study was approved by institutional review boards (Cedars-Sinai Institutional Review Board, IRBs 3358 and 23705, and UCLA Institutional Review Board IRB-11-001527). Written informed consent was obtained from all subjects by the Cedars-Sinai Medical Center. Separate informed consent was waived by UCLA IRB. All methods were carried out in accordance with relevant guidelines and regulations.

Inclusion criteria: IBD diagnosis was confirmed by Cedars-Sinai Medical Center gastroenterologists. The Cedars-Sinai Medical Center gastroenterologists referred the patients to surgical procedures, as medically indicated. These IBD patients mostly had severe disease activity or severe strictures after drug treatments that were justified for surgical resection. Colonic and mesenteric fat samples of IBD patients were collected during surgical removal of diseased tissues. Full-thickness involved regions of colonic tissues were used in this study. Colonic and mesenteric fat samples of control group patients were collected during surgical removal of colonic tumors and adjacent normal tissues. The colonic and mesenteric fat with normal histological structures were used as non-IBD control tissue samples. The presence of strictures in the colonic tissue was confirmed by the Cedars-Sinai Medical Center pathologists.

Exclusion criteria: Pregnant women, prisoners, or minors under age 18 were not included. Additionally, IBD patients with concurrent acute infection (CMV, C. difficile, and tuberculosis) and malignant conditions were excluded. Colonic and mesenteric fat samples of bad tissue quality or without significant proportions of mucosa were not included.

Serum Exosome Preparation

Serum exosomes were prepared by total exosome isolation reagent (#4478360, ThermoFisher) and quantified by bicinchoninic acid (BCA) protein assay (#23225, ThermoFisher).

ELISA

Human colonic tissues were homogenized in RIPA buffer with a protease inhibitors cocktail (sc-24948, Santa Cruz Biotechnology). Human sera were diluted ten-fold with reagent diluent and added to the ELISA plates. Elafin was detected with an ELISA kit (DY1747 R&D Systems) as described previously [26]. Serum cytokines were detected with multiplex ELISA (human 27-plex #m500kcaf0y, Bio-Rad).

Whole transcriptome RNA sequencing of human colonic RNA samples

Colonic total RNA samples from two stricturing CD and two non-stricturing CD patients (Cedars-Sinai Medical Center) were used for next-generation whole-transcriptome RNA sequencing (Omega Bioservices). Library was prepared by Illumina TruSeq Stranded mRNA library prep. Sequencing was run on HiSeq 4000/x Ten platform in PE 2x150 format with 5 million reads per sample.

Cell Cultures

Human CCD-18Co intestinal fibroblasts (ATCC) (2×10⁶ cells/plate) were cultured in minimal essential medium Eagle's medium (MEM) containing 10% fetal bovine serum (FBS) and 1% penicillin-streptomycin (P/S) (Invitrogen) [15, 25]. Serum-starved CCD-18Co cells were treated with 15 μg/ml of anti-elafin neutralizing antibody (AF1747, R&D Systems) or control antibody (AB-108-C, R&D Systems), followed by exposure to human sera from normal, UC, stricturing CD, and non-stricturing CD patients (100p1/mL). CCD18Co fibroblasts in MEM were incubated with 100 μg/ml of human serum exosomes for 24 hours. Human serum exosomes were obtained from 12 patients per group. For inhibition of miR-205-5p, serum-starved fibroblasts were pretreated with either 50 nM control (Y100199006) or miR205-5p (Y104101508-DDA) power inhibitors (Qiagen) for 24 hours. Power inhibitors were dissolved in Tris-EDTA buffer. The final concentration of miRNA inhibitor in cell culture was 50 μM.

Human primary intestinal fibroblasts (two CD patients) were isolated, as described previously [27]. The primary fibroblasts (1×10⁶ cells/plate) were cultured in Dulbecco's modified Eagle media (DMEM) containing 10% fetal bovine serum and 1% penicillin/streptomycin and serum-starved overnight before experiments [15].

Peripheral blood mononuclear cells (PBMCs) were obtained from a healthy donor (C-12907, Promocell). PBMCs in mononuclear cell medium (C-28030, Promocell) were incubated with 100 μg/mL of human serum exosomes for 24 hours. Human serum exosomes were obtained from 6 patients per group. At the end of the experiments, the treated PBMCs were centrifuged, and the cell pellets were used for RNA extraction.

Human mesenteric fat adipocyte experiments

Mesenteric fat preadipocytes from non-IBD, CD, and UC patients were collected from a previous study and stored in liquid nitrogen [28]. The human preadipocytes were thawed and cultured in DMEM/F12 media containing 10% calf serum and 1% P/S (Invitrogen) until >60% confluence was achieved. The preadipocytes were dissociated by trypsin/EDTA solution (Invitrogen) and seeded to 6-well plates (400,000 cells per plate) in DMEM/F12 media containing 10% calf serum and 1% P/S. Two days later, the preadipocytes underwent differentiation process by incubating with induction media (DMEM with FBS, P/S/G, bovine insulin (Sigma 1-5500; 1 μg/mL), dexamethasone (Sigma D-4902; 1 μM) and isobutylmethylxanthine (IBMX; Sigma 1-5500; 115 μg/mL) for two days, insulin media (DMEM with FBS, P/S/G and insulin (1 μg/mL)) for two days, and DMEM+FBS+P/S for two days [29]. The adipocytes were regarded as differentiated by the observation of lipid droplet deposition under microscope. The differentiated adipocytes were serum-starved for 8 hours, followed by incubation with human serum exosomes (100 μg/mL) for 16 hours. The conditioned cells were then switched to serum-free DMEM media for 6 hours to let the cells secrete elafin. The conditioned media were collected for elafin ELISA.

Histological Evaluation of Intestinal Injury

We prepared paraffin-embedded sections of each human colonic biopsies at UCLA Tissue Processing Core Laboratory (TPCL). Paraffin-embedded sections were cut at 4 μm thickness, and H&E staining of the tissue sections was performed as stated before [15, 25]. Microphotographs were recorded at multiple locations and blindly scored by two investigators using a previously described scoring system [30].

Elafin Immunohistochemistry

Elafin immunohistochemistry of human colonic and mesenteric fat tissues was performed by TPCL. Paraffin was removed with xylene. The sections were then rehydrated through graded ethanol. Endogenous peroxidase activity was blocked with 3% hydrogen peroxide in methanol for 10 minutes. Heat-induced antigen retrieval (HIER) was carried out for all sections in 0.01 M citrate buffer, pH=6 using a Biocare decloaker at 95° C. for 25 minutes. After treatment with blocking buffer (2% BSA) for 1 hour, the slides were then incubated overnight at 4° C. with rabbit polyclonal to elafin in 2% BSA at 1:100 dilution (Sigma, HPA017737). The signal was detected using the rabbit horseradish peroxidase EnVision kit (DAKOCytomation, K4003). This secondary antibody kit was directly applied to the slides without dilution. All sections were visualized with the diaminobenzidine reaction and counterstained with hematoxylin. Images were taken with a Zeiss AX10 microscope in a blind manner.

Quantitative Real-Time RT-PCR

Total RNA was isolated by an RNeasy kit (#74104, Qiagen) and reverse transcribed into cDNA by a high-capacity cDNA RT kit (#4368813, ThermoFisher). Quantitative PCR reactions were run with Fast Universal PCR master mix (#4352042, ThermoFisher) in a Bio-Rad CFX384 system [26]. The mRNA expression was determined by using cataloged primers (ThermoFisher) for human collagen COL1A2 (Hs01028956_m1), alpha smooth muscle actin ACTA2 (Hs00426835_g1), transforming growth factor-beta one TGF-b1 (Hs00998133_m1), and elafin P13 (Hs00160066_m1). Relative mRNA quantification was performed by comparing test groups and normal control group, after normalization with endogenous control gene human 18S (Hs99999901_s1).

Preliminary screening for the presence of serum exosomal miRNAs was determined using a miScript human miFinder PCR array (MIHS-001Z, Qiagen). RNA was converted to cDNA using miScript RT kit (218060). PCR reactions were performed with miScript SYBR Green PCR kit (218073). Since many miRNAs in the PCR arrays were undetectable in serum exosomes, we selected the detectable miRNAs and determined their relative expression using miRCURY LNA miRNA PCR assays. RNA was converted to cDNA using miRCURY LNA RT kit (339340) and PCR reactions were run with miRCURY LNA SYBR Green PCR kit (339346). The miRNA expression was detected using Qiagen miRCURY PCR assays. Relative miRNA quantification was performed by comparing test groups and normal control group, after normalization with housekeeping miRNA (RNU1A1). The measurement of miRNA was determined by miRCURY LNA PCR assays. All miRNA-related reagents were purchased from Qiagen.

The fold changes are expressed as 2ΔΔt. Fold-change values greater than one indicate a positive- or an up-regulation, and the fold-regulation is equal to the fold-change. Fold-change values less than one indicate a negative or down-regulation, and the fold-regulation is the negative inverse of the fold-change.

Power Analysis

Serum sample study: At least 30 patients per group were required to achieve a statistically significant difference of serum elafin levels between control (7939 pg/ml), UC (12987 pg/ml), and CD (12344 pg/ml) patients with standard deviation=4860, alpha=0.5, and power=0.8. The combined dataset from the two serum cohorts satisfied this requirement.

Surgical sample study: At least 30 patients per group were required to achieve a statistically significant difference of colonic elafin mRNA expression between control (1.39 fold), UC (12 fold), and CD (4 fold) patients with standard deviation=2.55, alpha=0.5, and power=0.8. Our cohort satisfied this requirement.

Statistical Analysis

Colonic elafin mRNA and protein expression were arranged in low-to-high order. The entire range of data was divided into three equal tertiles (⅓, ⅓, ⅓). Serum elafin concentrations were arranged in order from low to high. We compared the performance of multiple cut-off points of elafin levels at each disease parameter for optimization of test performance. After many calculations using various cutoff points, the optimized universal cut-off points yielding the highest area under the curve (AUC) values in receiver operating characteristic (ROC) curves (most accurate) were shown in this study. Calculation of prevalence of the disease, sensitivity, specificity, positive predictive value, negative predictive value, and relative risk was described previously [10]. AUCs of ROC curves were calculated online (easyROC web-tool, www.jrocfir.org, and Microsoft Azure Machine Learning Studio). Unpaired Student's t-tests were used for two-group comparisons of continuous data (GraphPad QuickCalcs) online. One-way ANOVAs with Tukey Honestly Significant Difference post-hoc tests were used for multiple-group comparisons (Statpages) online. Bar graphs and scatter plots were made using Microsoft Excel. Results were expressed as mean+/−SEM. Significant p values are shown in each figure.

Machine Learning Algorithm for Indicating The Presence of Stricturing in CD Patients

The combined CD cohort dataset containing 67 CD patients in CSV file format was loaded into the Microsoft Azure Machine Learning Studio. The dataset included serum elafin level and 14 clinical parameters, i.e., patient's age at blood collection (number), years of disease duration (number), C-reactive protein/CRP (number), ESR (number), HBI (number), count of IBD-related surgery (number), gender (male or female), smoking habit (yes or no), use of biologics (yes or no), use of steroid (yes or no), use of immunomodulator (yes or no), use of aminosalicylate (yes or no), presence of fistula (yes or no), and presence of stricture (yes or no).

The machine learning algorithm included the clinical data based on their relevance for the accurate indication of intestinal strictures. The entire dataset was split into 50% for training and 50% for evaluation. The trained model was built on a two-class decision forest algorithm. The algorithm utilized default parameters including bagging resampling method, single parameter create trainer mode, 8 decision trees, 32 maximum depth of the decision trees, 128 random splits per node, and 1 minimum number of samples per leaf node. The scored dataset showed score probability (0-0.5 indicates no stricture, 0.51-1.0 indicates stricture), scored labels (yes or no stricture), and AUC values of ROCs.

Results

High Serum Elafin Levels Indicated an Elevated Risk of Stricture in CD Patients

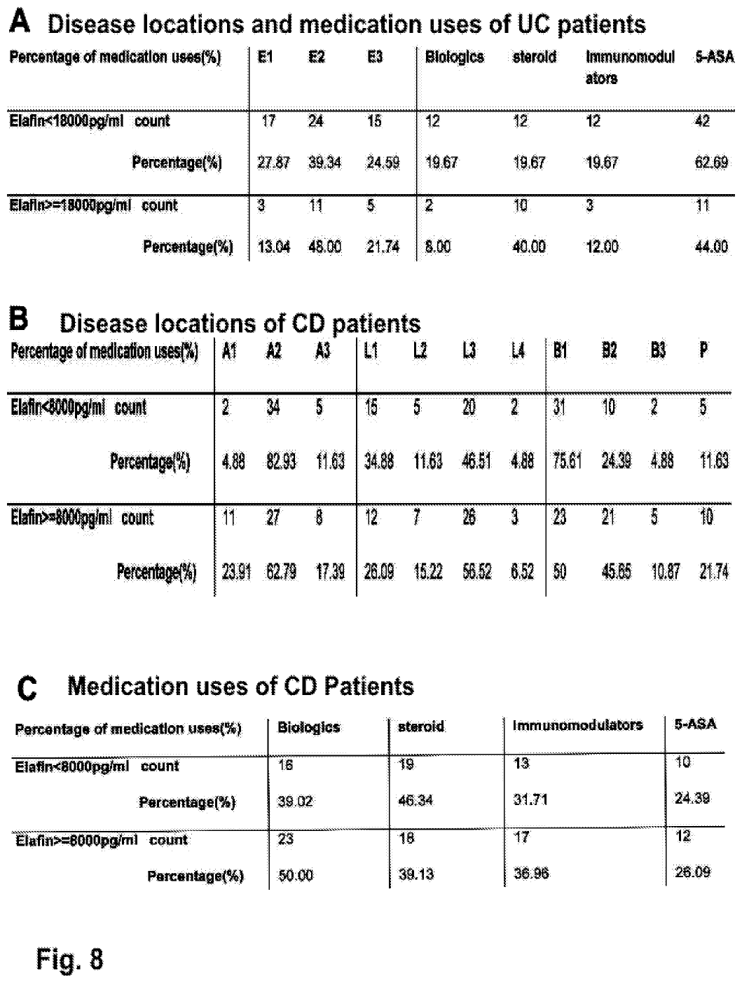

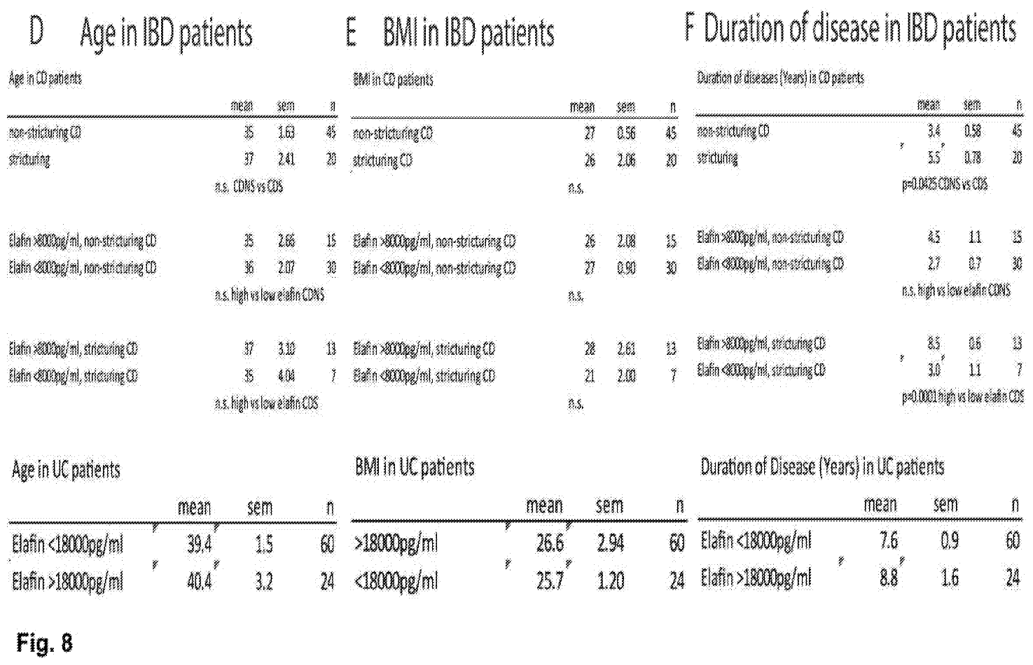

Baseline characteristics, disease locations, and medication use of the two serum sample cohorts are shown in 51 Table (FIG. 7). Demographic profiles, disease conditions, and medication uses of these two cohorts are comparable, but not the same. All IBD patients were not treatment naïve, but 20% of UC patients and 17% of CD patients did not have concurrent medication at the time of blood collection. Medication use statistics are shown in S2 Table (FIG. 8). Our study (80 UC and 95 CD) included more IBD patients than several other antimicrobial peptide-IBD studies [21, 31, 32].

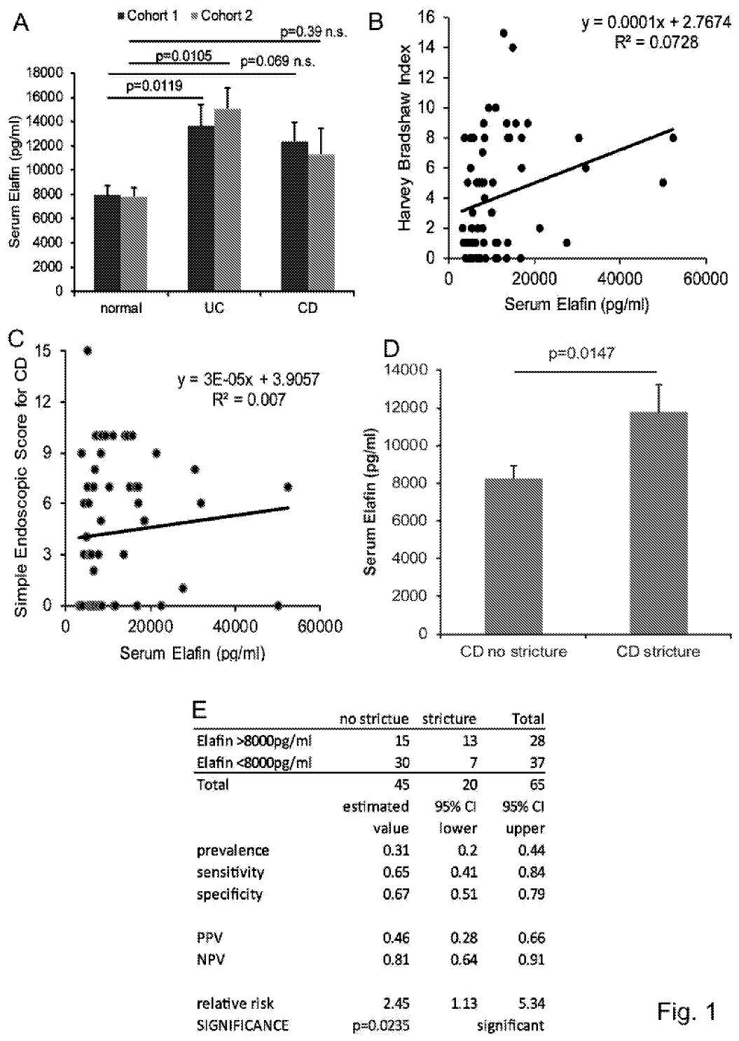

The detected serum elafin levels in nanogram per milliliter range were similar to the findings of other elafin-related studies [17, 33]. UC patients in both cohorts had significantly higher serum elafin levels than control patients (FIG. 1A). There was a trend of mildly increased serum elafin levels in CD patients, but the difference was not statistically significant (FIG. 1A). We combined the datasets of both cohorts to yield a sample size for statistical analysis. The combined dataset had also been used in our previous cathelicidin-IBD biomarker study [10]. Serum elafin levels were directly proportional to the Harvey Bradshaw Index (HBI) in CD patients (FIG. 1B), but linear regression suggests a weak positive correlation (low R2 value). Serum elafin levels were not associated with the Simple Endoscopic Score for Crohn's Disease (SES-CD) (FIG. 1C).

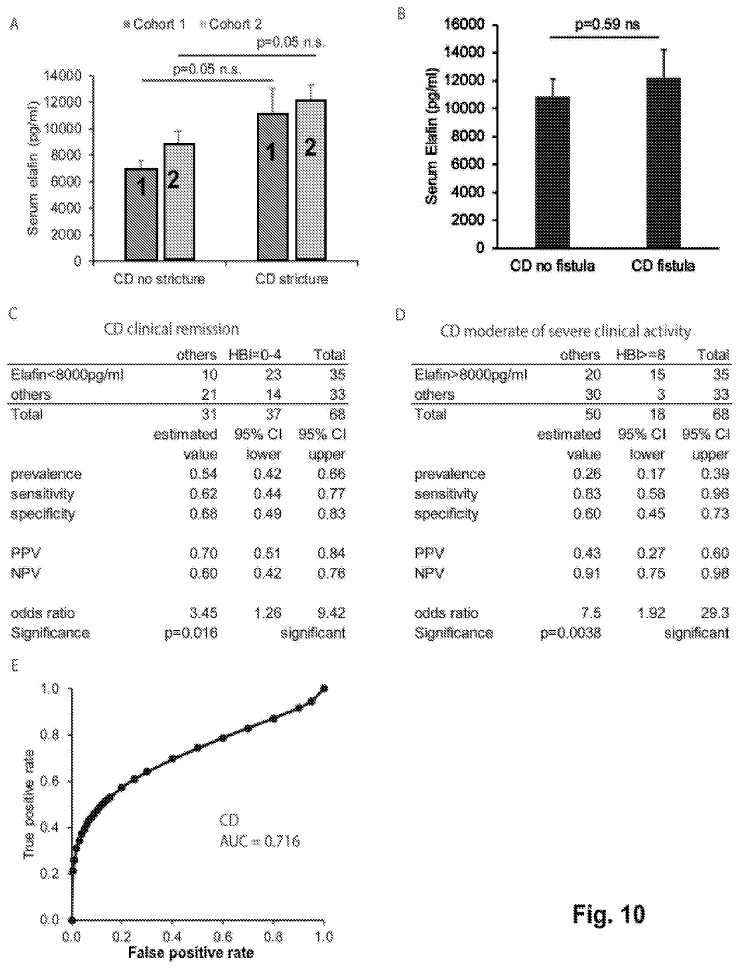

Cohort 1 had 50% stricturing CD patients, while cohort 2 had 24% stricturing CD patients (51 Table; FIG. 7). The number of stricturing CD patients in individual cohorts was below the required sample size to achieve statistical significance. Separate calculations of individual cohorts showed increased serum elafin levels in stricturing CD patients, but the differences were statistically insignificant (FIG. 10A). The combined dataset shows that stricturing CD patients had significantly higher serum elafin levels than non-stricturing CD patients (FIG. 1D). The high elafin group had a significantly higher relative risk (RR=2.45) than the low elafin group in having intestinal strictures at the time of blood collection (FIG. 1E). However, the serum elafin levels in CD patients with and without fistulas were similar, suggesting that serum elafin levels are not associated with the occurrence of fistulas in CD patients (FIG. 10B).

We found no association between elafin levels, age (A1-3), disease location (L1-4), use of medication, and body mass index (BMI) at the time of blood collection (S2 Table, FIG. 8, panel A-E). However, stricturing CD patients had significantly longer duration of disease than non-stricturing CD patients did (S2 Table, FIG. 8, panel F). Among stricturing CD patients, the high serum elafin group also had significantly longer duration of disease than the low serum elafin group (S2 Table, FIG. 8, panel F).

Machine Learning Algorithm Improves the Accuracy of Elafin for Indicating Strictures in CD Patients

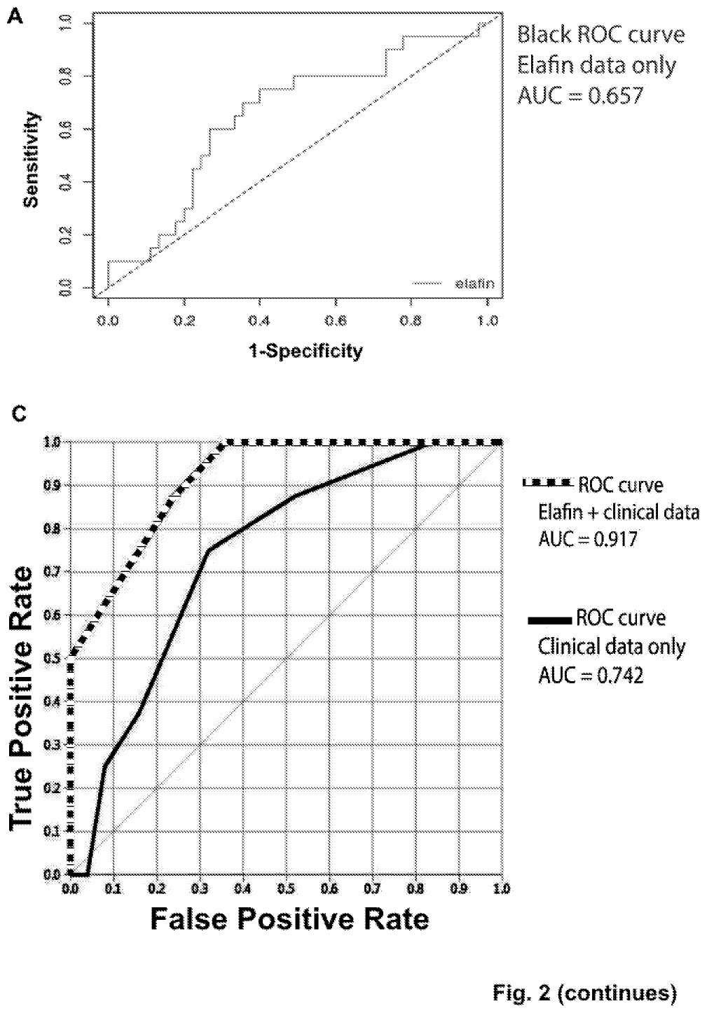

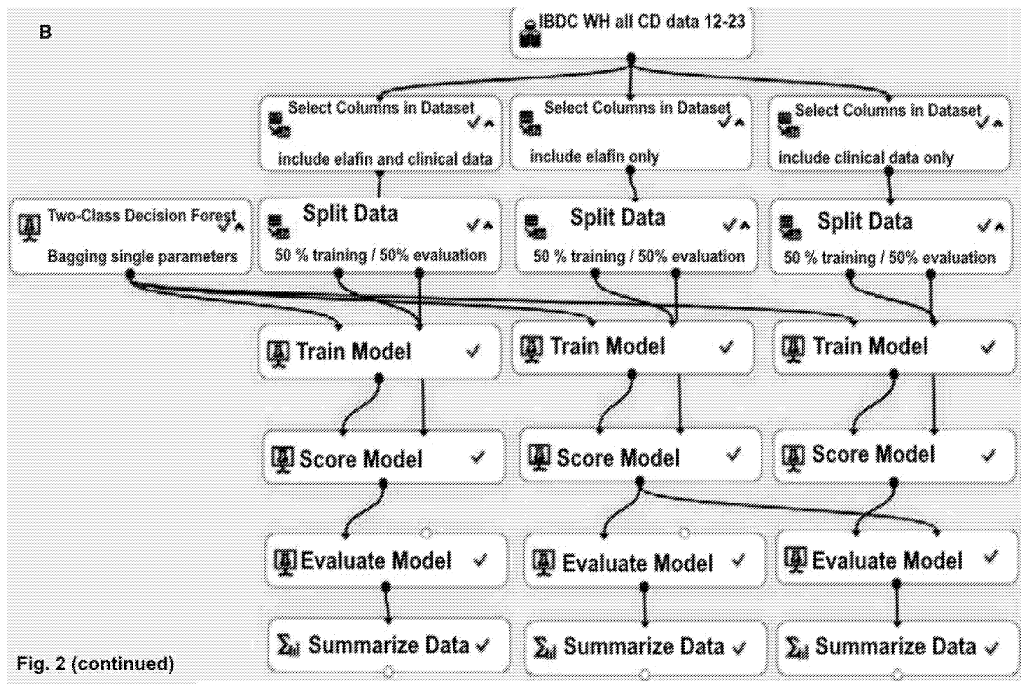

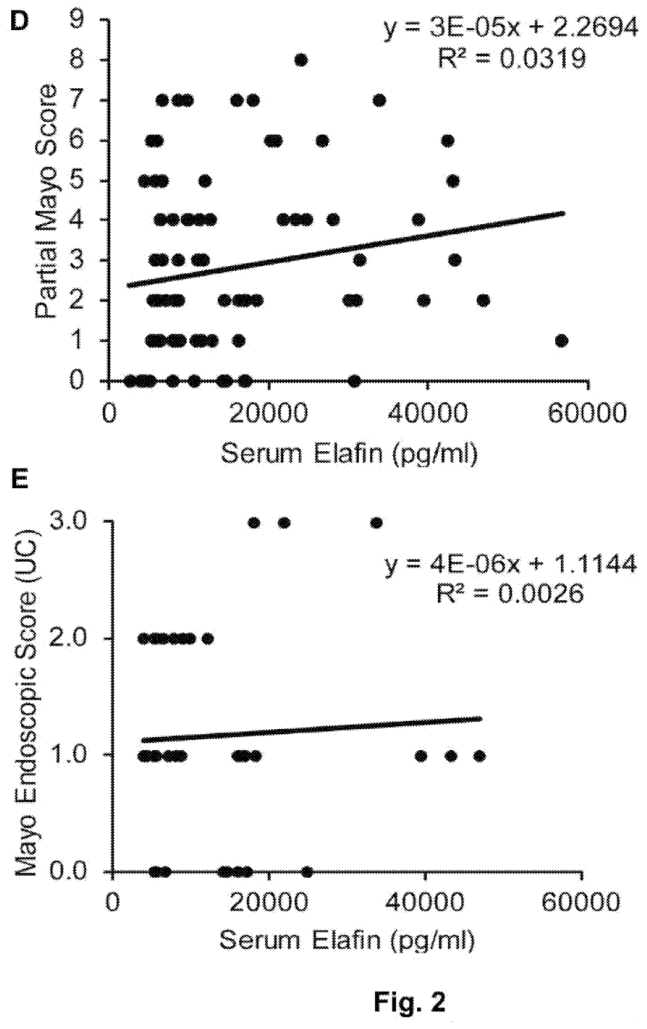

To evaluate whether circulating elafin alone is a good indicator for intestinal stricture, we determined its accuracy with ROC analysis. Serum elafin alone is moderately accurate for indicating stricture in CD patients (area under curve/AUC=0.657 using elafin alone) (FIG. 2A). We utilized machine learning to develop an algorithm for indicating the presence of intestinal strictures in CD patients (FIG. 2B). The optimized trained model using serum elafin levels and commonly available clinical data together is much more accurate than those using either elafin or clinical data alone (AUC=0.917 using combined data; 0.742 using clinical data alone) (FIG. 2C). Therefore, a combination of high serum elafin level and other characteristics are strongly associated with the presence of stricture in CD patients.

Serum Elafin Levels are Not Correlated with Endoscopic Disease Activity in UC Patients

High serum elafin levels had a weak positive correlation with increased Partial Mayo

Score (PMS) in UC patients, as indicated by low R2 value (FIG. 2D). Serum elafin levels had no association with the Mayo Endoscopic subscore in the same set of UC patients (FIG. 2E). There was no association found between elafin levels, disease location (E1-3), use of medication, age, BMI, and duration of disease at the time of blood collection (S2 Table, FIG. 8, panel A, D-F).

Colonic Elafin mRNA and Protein Expression Were Low in Stricturing CD Patients

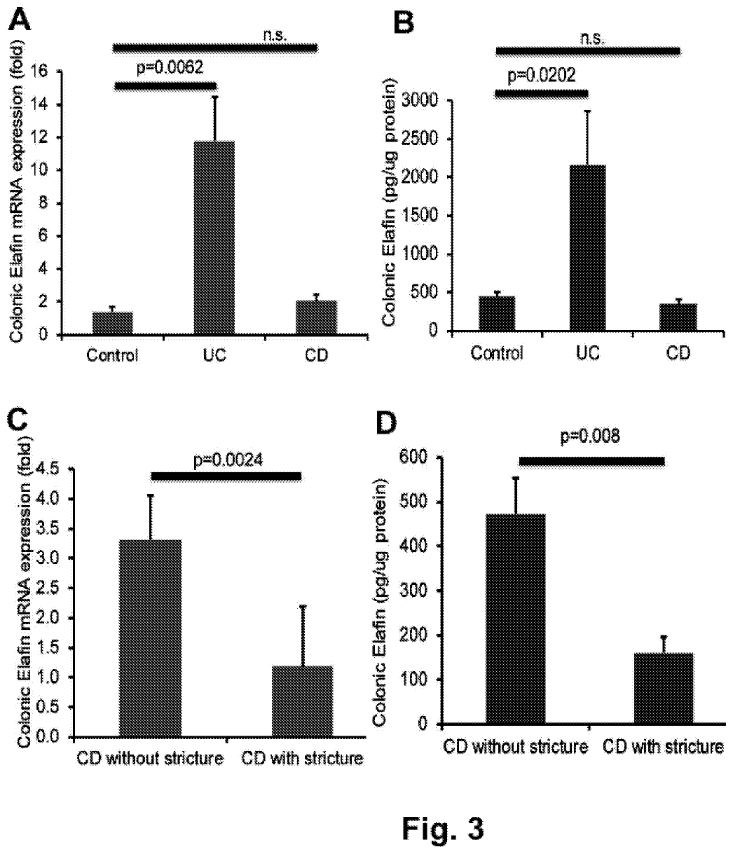

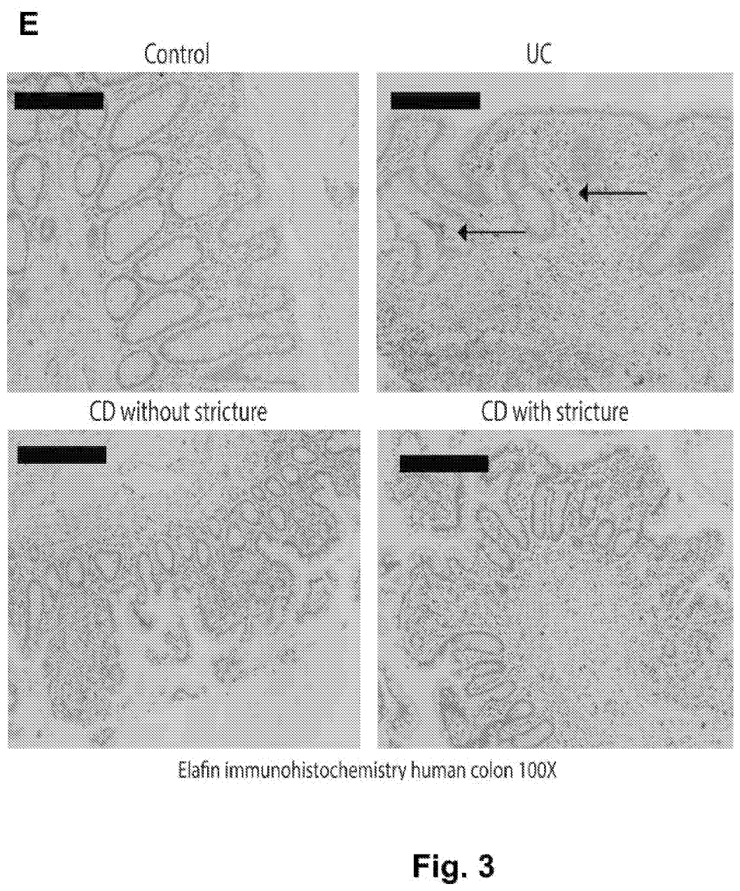

Baseline characteristics of the colonic tissue cohort are shown in S3 Table, FIG. 9, panel A [25]. Consistent with a previous study [19], UC patients had significantly higher colonic elafin mRNA and protein expression than control non-IBD patients (FIG. 3A and 3B). CD patients with stricture had significantly lower colonic elafin mRNA and protein expression than those without stricture (FIG. 3C and 3D).

Colonic elafin protein expression in the control patients was weak (FIG. 3E). Elafin immunoreactivity was found in the colonic mucosa and lamina propria of UC patients (FIG. 3E). Consistent with another study [20], colonic elafin protein expression was low in CD patients with and without stricture (FIG. 3E).

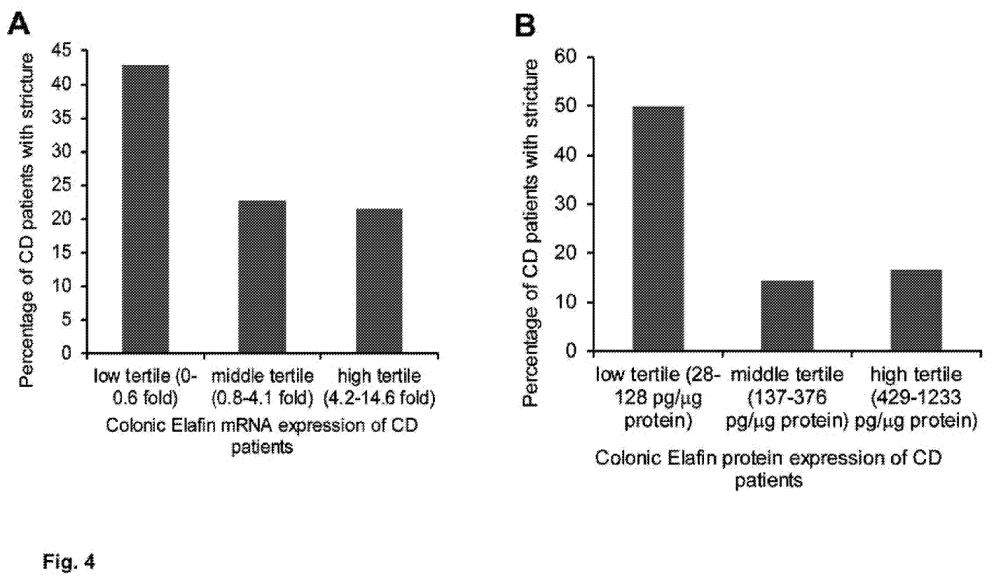

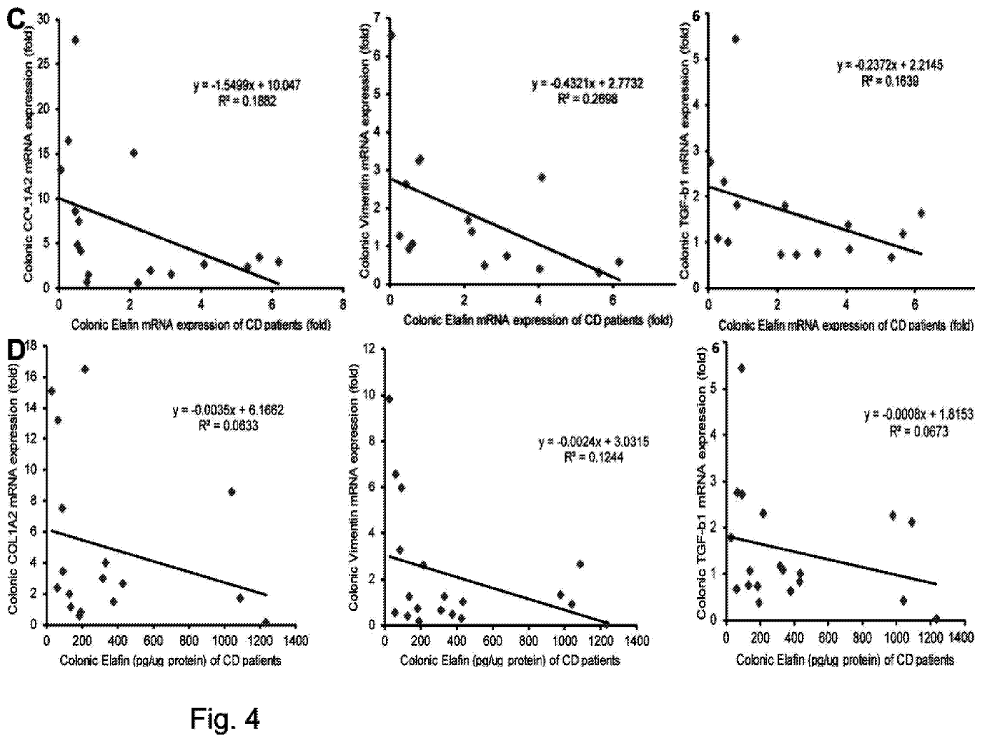

When the entire CD patient cohort was divided into tertiles of colonic elafin mRNA and protein expression, the low tertile tended to have a higher incidence of intestinal stricture than the middle and high tertiles (FIG. 4A and 4B). This evidence suggests that stricturing CD patients have very low colonic elafin expression. Similarly, we found that colonic elafin mRNA and protein expression have a modestly negative correlation with colonic fibrogenic factors (COL1A2, VIM, and TGF-b1) mRNA expression in CD patients (FIG. 4C and 4D). Therefore, stricturing CD patients with increased colonic fibrogenic factor expression have low colonic elafin expression.

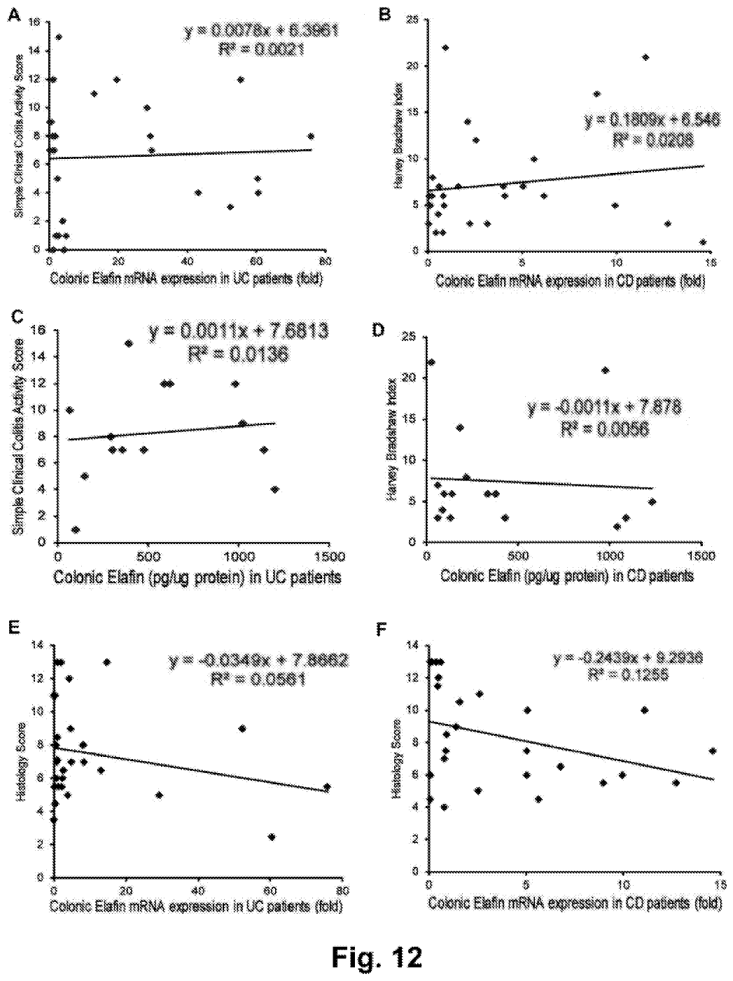

Colonic elafin mRNA and protein expression were not associated with clinical disease activity in UC and CD patients (FIG. 12A-D). Colonic elafin mRNA expression had a modest negative correlation with histology score of the colonic tissues in UC and CD patients (FIG. 12E-F). The colonic elafin mRNA expression or the presence of intestinal strictures had no association with current use of anti-TNF medication, current use of steroid or 6-mercaptopurine, gender, BMI, age at biopsy collection, or duration of diseases in both UC and CD patients (S3 Table, FIG. 9, panel B-C).

Mesenteric Fat Adipocytes Are a Source of Circulating Elafin in Stricturing CD Patients

l Since stricturing CD patients had high serum elafin levels and low colonic elafin expression, we continued to discover the source of elafin. Stricturing CD patients have a higher visceral to subcutaneous fat area ratio than non-IBD patients [34]. Stricturing CD patients also have a higher visceral fat/total fat mass ratio than non-stricturing CD patients [35]. Mesenteric fat may be a potential source of elafin in circulation.

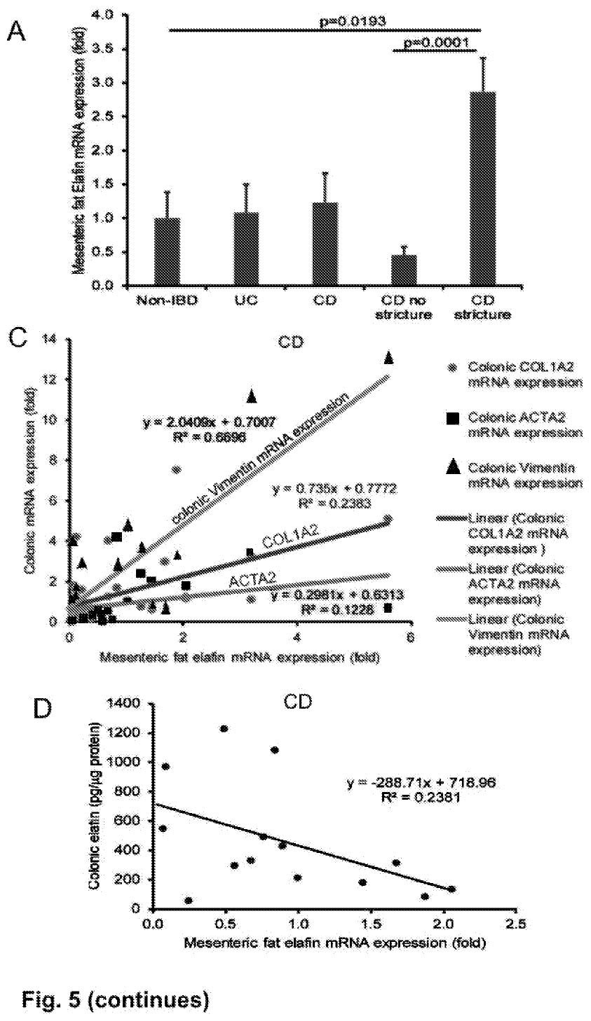

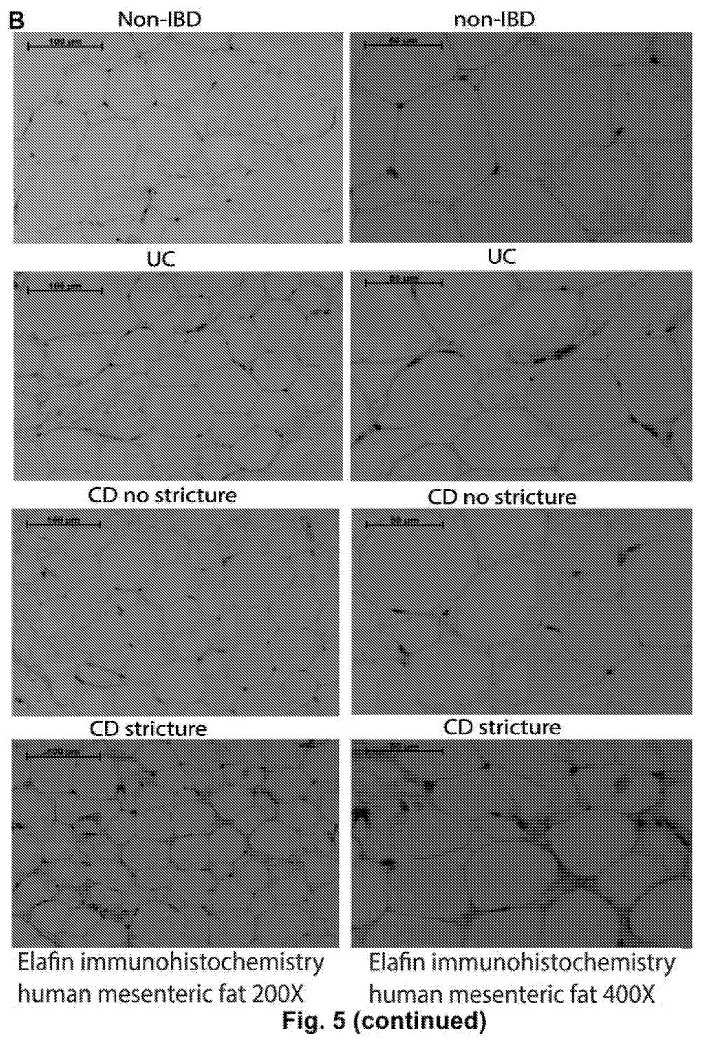

Sticturing CD patients had significantly higher mesenteric fat elafin mRNA expression than control and non-stricturing CD patients (FIG. 5A). Immunohistochemistry indicates that elafin protein expression in mesenteric fat of stricturing CD patients was much higher than those in non-IBD, UC, and non-stricturing CD patients (FIG. 5B, left side). At high magnification (400×), the elafin-positive signal is located around adipocytes of stricturing CD patients (FIG. 5B, right side).

Interestingly, our patient-matched biopsy collection indicates that mesenteric fat elafin mRNA expression is positively correlated with the mRNA expression of colonic fibrogenic factors (COL1A2, ACTA2, VIM) and negatively correlated with the colonic elafin protein expression in CD patients (FIG. 5C and 5D). Therefore, increased elafin expression in mesenteric fat is associated with low elafin expression and intestinal strictures in CD patients.

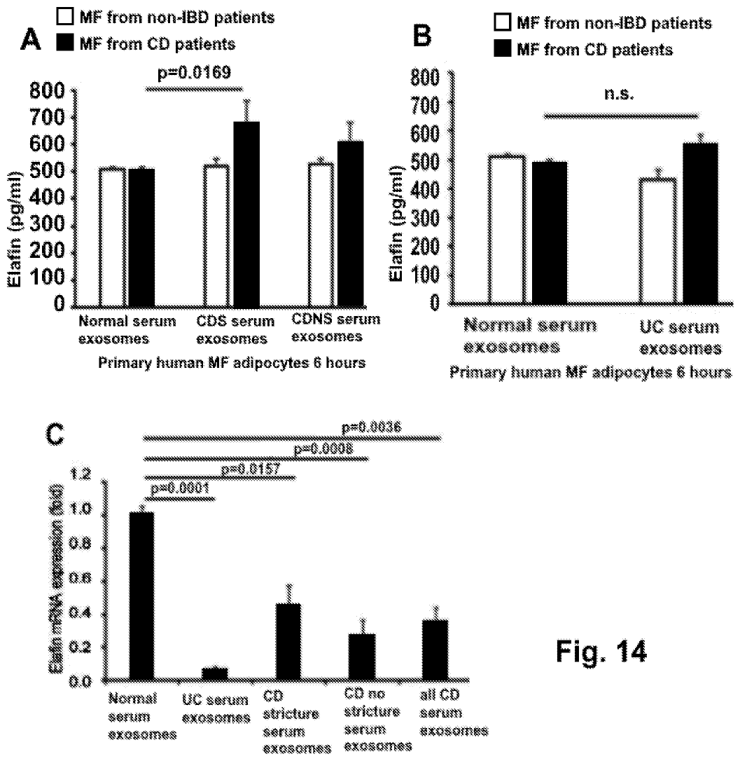

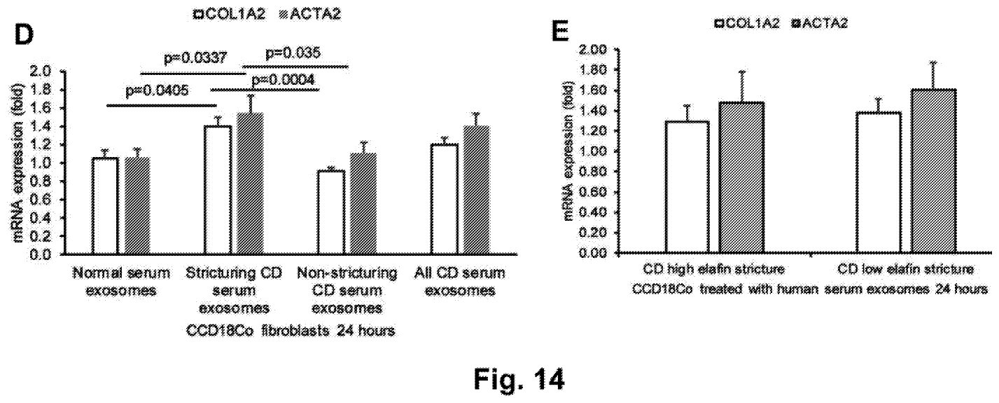

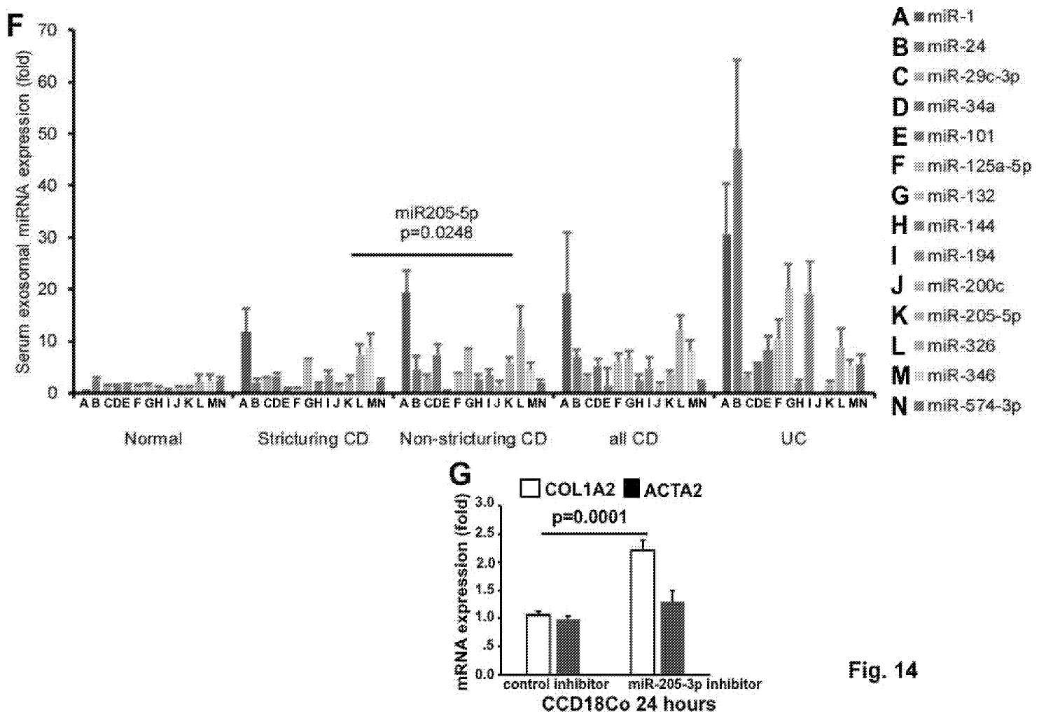

To identify the link between intestinal strictures and adipose-derived elafin expression, we exposed primary human differentiated mesenteric fat adipocytes to human serum exosomes and determined their elafin secretion. Circulating exosomes mediate long-distance communication between organs and affect disease activity in IBD [36]. When the adipocytes from non-IBD, CD, and UC patients were exposed to normal serum exosomes, their elafin secretion was similar (FIG. 14A-B). Serum exosomes from stricturing CD patients, but not non-stricturing CD patients, significantly increased elafin secretion of the adipocytes from CD patients (S5A FIG.), while serum exosomes from UC patients did not affect elafin secretion of the adipocytes from UC patients (FIG. 14B). Therefore, differentiated mesenteric fat adipocytes are a source of elafin in the stricturing CD patients.

Elafin Induces Fibrogenesis in Human Colonic Fibroblasts

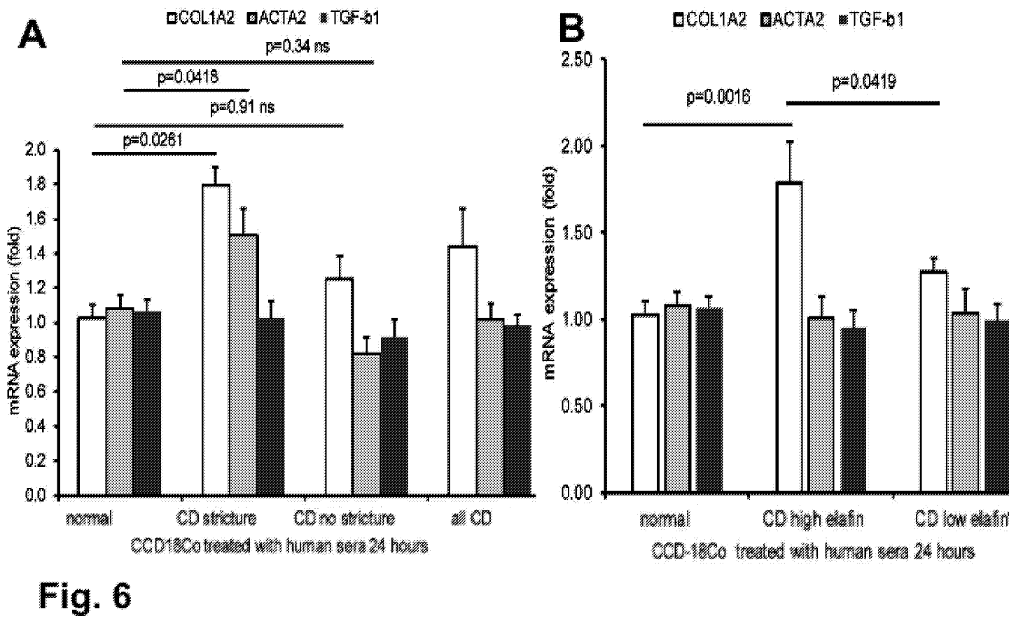

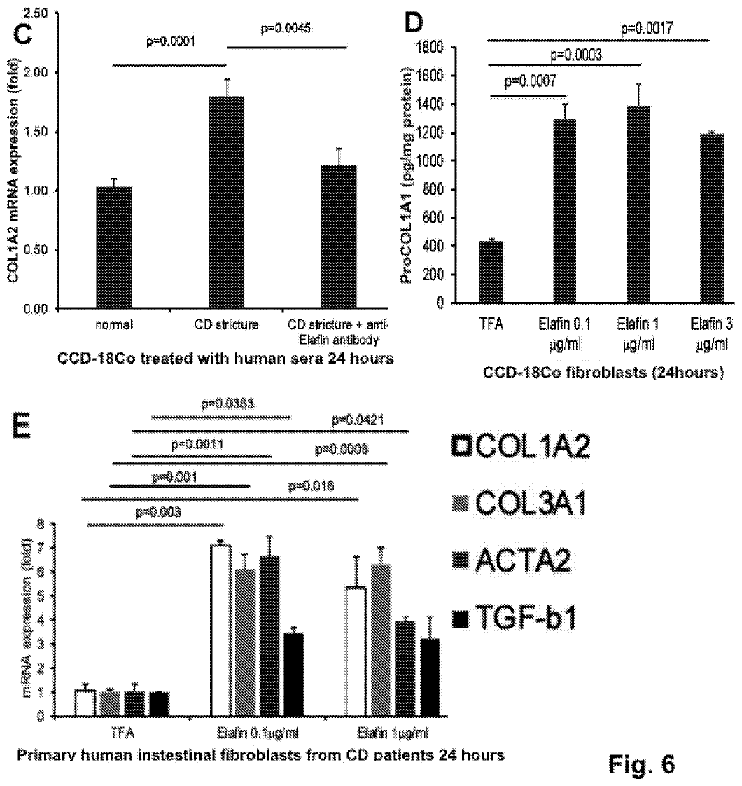

To determine whether the circulating elafin regulates fibrogenesis, we treated the human colonic CCD-18Co fibroblasts with human sera from normal, stricturing CD, and non-stricturing CD patients (FIG. 6A). Sera from stricturing CD patients, but neither from healthy control nor non-stricturing CD patients, significantly increased collagen and ACTA2, but not TGF-b1, mRNA expression in the CCD-18Co fibroblasts (FIG. 6A). Exposure to sera from high elafin CD patients also significantly increased COL1A2, but not ACTA2 and TGF-b1, mRNA expression in CCD-18Co fibroblasts (FIG. 6B). Neutralization of elafin with anti-elafin antibody partially reduced the increased collagen mRNA expression in fibroblasts exposed to sera from stricturing CD patients (FIG. 6C).

Elafin significantly increased pro-collagen 1A1 protein expression in CCD-18Co fibroblasts (FIG. 6D) and increased collagen (COL3A1 and COL1A2), ACTA2, and TGF-b1 mRNA expression in primary human intestinal fibroblasts from CD patients (FIG. 6E), suggesting that elafin mediates direct pro-fibrogenic effects on human intestinal fibroblasts.

Discussion

This report indicates that circulating elafin is associated with intestinal strictures in CD patients. Some of the non-stricturing CD patients also had high circulating elafin levels, leading to moderate accuracy when only elafin was used in identifying stricturing CD patients. Elafin alone is insufficient to indicate intestinal strictures accurately due to the complexity of the patients' many clinical characteristics not taken into consideration. Machine learning improved the accuracy of identifying the presence of intestinal fibrosis among CD patients. Machine learning, a branch of artificial intelligence, is increasingly important for IBD biomarker discovery and disease activity prediction [37, 38]. We have included the Tune Model Hyperparameters module during the tuning and cross-validation step, so the current algorithm has the highest accuracy. The ensemble model of a decision forest worked by voting on the most popular output class of multiple decision trees. Bagging also reduced the chance of overfitting complex models. The resulting algorithm should have high robustness and generalizability. Our predictive experiment is now available on Microsoft Azure Al Gallery (https://gallery.cortanaintelligence.com/Experiment/Use-elafin-and-clinical-data-for-indicating-stricture-Predictive-Exp) for test-run on Microsoft Azure Machine Learning Studio (Classic).

Two expert panels had attempted to establish consensus endpoints and criteria for diagnosis and response to therapy in stricturing CD [39, 40]. Diagnostic approaches for intestinal strictures are based on radiological and endoscopic assessment, which are inherently inconvenient and expensive. The current imaging assessments, including CT and MRE, are unable to differentiate inflammatory versus fibrotic strictures, while some strictures are inaccessible to endoscopy [6]. We are uncertain whether elafin expression is different between inflammatory strictures, fibrotic strictures, and mixed strictures. However, elafin, as a minimally invasive circulating biomarker, may be suitable for identifying high-risk stricturing CD patients for further evaluation. We expect elafin can be used to predict future development of intestinal strictures.

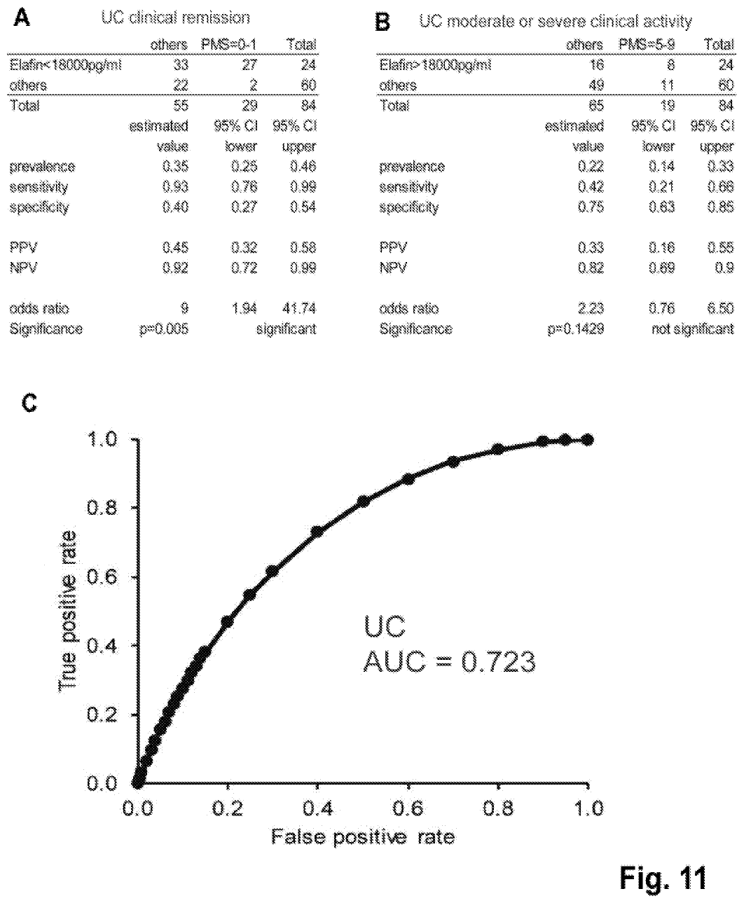

Circulating elafin has moderate sensitivity and specificity in indicating clinical disease activity in CD and UC patients (FIGS. 10C, 10D, 11A and 11B). The accuracies of elafin in indicating clinical disease activities (AUC=0.716 in CD and AUC=0.723 in UC) were similar to the accuracy of CRP (AUC=0.63 in CD and AUC=0.70 in UC) (FIGS. 10E and 11C) [10]. Our biobank is continuing to collect samples and monitor the disease activity of IBD patients.

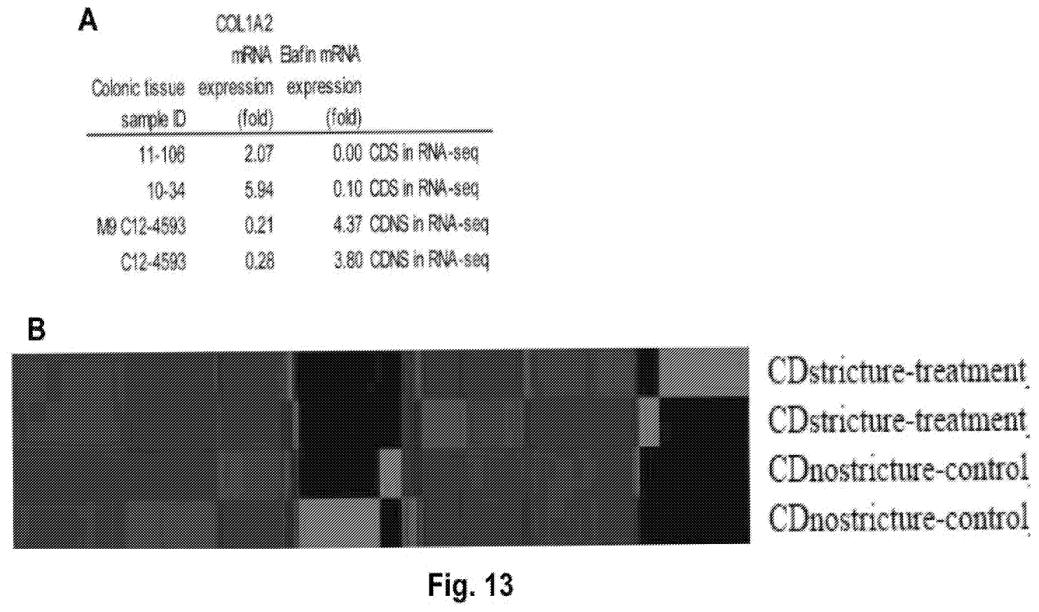

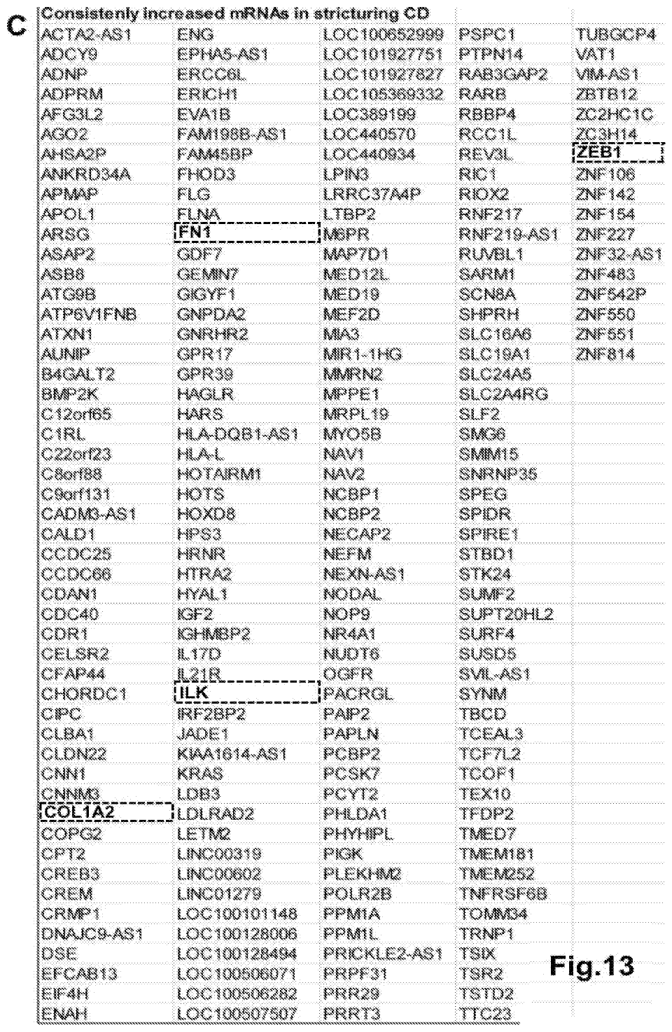

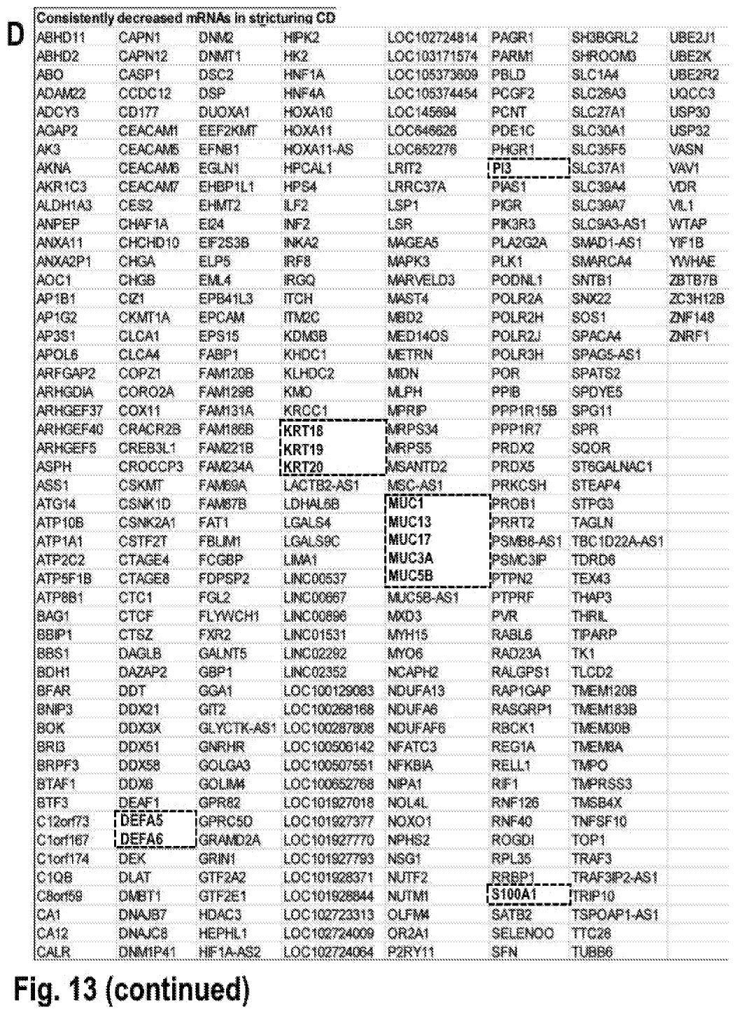

To understand the significance of reduced colonic elafin expression in stricturing CD patients, we determined the gene signature in intestinal fibrosis using whole-transcriptome RNA sequencing (RNA-seq). High collagen COL1A2 mRNA expression in the stricturing CD colonic tissue samples indicated fibrosis (FIG. 13A). Heat map indicated that the colonic gene expression patterns of stricturing CD and non-stricturing CD patients were different (FIG. 13B). Intestinal stricture affected the expression of —800 genes in CD patients, indicating the specific intestinal host responses to strictures (FIG. 13B). Notably, stricturing CD patients consistently had increased fibrosis-related genes such as collagen (COL1A2) and fibronectin (FN1), suggesting that the fibrotic intestinal tissues were occupied by extracellular matrix (FIG. 13C, upper panel).

On the other hand, stricturing CD patients had low expression of epithelial cell-related genes such as keratin (KRT), mucin (MUC), and solute carrier SLC superfamily (FIG. 13C, lower panel), suggesting impaired epithelial functioning. Colonic expression of antimicrobial peptide genes such as elafin (P13) and alpha defensin (DEFAS-6) was also consistently low in stricturing CD patients (FIG. 13C, lower panel). Based on these findings, reduced colonic elafin mRNA expression is associated with impaired functioning of the colonic mucosa in the stricturing CD patients.

Colonic mucosa of UC patients has increased antimicrobial peptide expression, such as cathelicidin [31] and beta-defensin 2 [41]. This response may be a protective mechanism against the invasion of luminal bacteria [12, 42, 43]. Since neutrophil accumulation is commonly observed in the colonic mucosa of UC patients [44], the contribution of neutrophil-derived elafin may increase colonic elafin expression, which possibly regulates neutrophil elastase activity and tissue damage in UC patients [45]. Intestinal stricture development involves multiple CD-specific factors. Many UC patients have increased elafin expression (FIGS. 1A, 3A and 3B), but none of them develop intestinal strictures.

This Example supports the association between adipose tissue and stricture development [34, 35]. The increased mesenteric fat elafin production may be an attempt to compensate for the down-regulated colonic elafin expression by raising circulating elafin levels in the stricturing CD patients (FIG. 5). We discovered that serum exosomes from stricturing CD patients induced elafin secretion in mesenteric fat adipocytes from CD patients (FIG. 14A), but the exosomal elafin-inducing factors are unknown.

A previous study demonstrated that peripheral blood leukocytes from IBD patients had reduced elafin mRNA expression [21, 22]. Similarly, exposure to serum exosomes from stricturing CD, non-stricturing CD, and UC patients significantly reduced elafin mRNA expression in peripheral blood mononuclear cells (PBMCs) from normal subjects (FIG. 14C). PBMCs are not a significant source of circulating elafin in IBD patients.

Serum exosomes from stricturing CD patients induced COL1A2 and ACTA2 mRNA expression in CCD-18Co fibroblasts (FIG. 14D). The pro-fibrogenic effects of these serum exosomes was not affected by the circulating elafin levels of the stricturing CD donors (FIG. 14E). Therefore, serum exosomes are an elafin-independent pro-fibrogenic factor in CD patients.

Approximately 83-99% of circulating miRNAs are carried by serum exosomes [46]. Therefore, exposure of fibroblasts, adipocytes, or PBMCs to serum exosomes from IBD patients may mimic the circulating environment in IBD. Stricturing CD patients had significantly lower serum exosomal miR205-5p expression than non-stricturing CD patients (FIG. 14F). Inhibition of miR205-5p induced COL1A2 mRNA expression in CCD-18Co fibroblasts (S5G Fig). The low serum exosomal miR205-5p expression may be associated with the pro-fibrogenic effect of circulating exosomes from stricturing CD patients because miR205-5p is anti-fibrogenic [47]. The current analysis of serum exosomal miRNAs was limited and did not find the correlations between the tested miRNAs and circulating elafin levels. We will perform RNA sequencing and proteomic analysis of serum exosomes in the future.

Circulating elafin is not associated with the endoscopic severity of colitis in CD and UC patients (FIGS. 1C and 2E) because colonic elafin expression is not strongly associated with mucosal histology scores in CD patients (FIG. 12E). The low colonic elafin expression did not affect the mucosal histology scores in CD patients (FIG. 12F). However, it is unfeasible to evaluate the influence of endogenous elafin in the development of intestinal fibrosis in mice because they do not have the elafin gene.

Conclusions

The study reported in this Example is the first to recognize elafin as a communication signal between mesenteric fat, blood, and intestine during stricture development. Intestinal stricture is associated with increased circulating elafin levels, reduced intestinal elafin expression, and increased mesenteric fat elafin expression. Machine learning integrated the elafin level and clinical data to develop an improved algorithm for indicating the presence of intestinal strictures in CD patients accurately.

Elafin test may be an adjunct to currently available modes of investigation in CD patients in general. Gastroenterologists need to assess clinical disease activity (HBI) and have required clinical data ready as in current clinical practice. If there is a suspicion of the presence of intestinal strictures in the CD patients, we suggest including elafin tests in regular blood tests along with CRP and ESR during clinical visits of CD. With the required data, the machine-learning algorithm calculates score probability instantly. If the score probability is >0.5, further diagnosis of intestinal strictures, such as endoscopy or imaging, is recommended.

### REFERENCES

1.Kugathasan S, et al. Lancet. 2017; 389(10080):1710-8.
2. Chan W P W, et al. J Gastroenterol Hepatol. 2018; 33(5):998-1008.
3. Graham M F, et al. Gastroenterology. 1988; 94(2):257-65.
4. Louis E, et al. Gut. 2001; 49(6):777-82.
5. Cleynen I, et al. Gut. 2013; 62(11):1556-65.
6. Bettenworth D, et al. World J Gastroenterol. 2016; 22(3):1008-16.
7. Lenti M V, Di Sabatino A. Mol Aspects Med. 2019; 65:100-9.
8. Giuffrida P,et al. United European Gastroenterol J. 2016; 4(4):523-30.
9. Mosli M H, et al. Am J Gastroenterol. 2015; 110(6):802-19.
10. Tran D H, et al. BMC Gastroenterol. 2017; 17(1):63.
11. Walsham N E, Sherwood R A. Clin Exp Gastroenterol. 2016; 9:21-9.
12. Ho S, Pothoulakis C, Koon H W. Curr Pharm Des. 2013; 19(1):40-7.
13. Hing T C, et al. Gut. 2013; 62(9):1295-305.
14. Tai E K, et al. Exp Biol Med (Maywood). 2007; 232(6):799-808.
15. Yoo J H, et al. Cell Mol Gastroenterol Hepatol. 2015; 1(1):55-74.
16. Shaw L, Wiedow O. Biochem Soc Trans. 2011; 39(5):1450-4.
17. Olewicz-Gawlik A, et al. Hum Immunol. 2017; 78(9):559-64.
18. Elgharib I, et al. Int J Dermatol. 2019; 58(2):205-9.
19. Flach C F, et al. Inflamm Bowel Dis. 2006; 12(9):837-42.
20. Schmid M, et al. J Leukoc Biol. 2007; 81(4):907-15.
21. Zhang W, et al. Inflamm Bowel Dis. 2017; 23(12):2134-41.
22. Zhang W, et al. Zhonghua Yi Xue Za Zhi. 2016; 96(14):1120-3.
23. Muto J, et al. J Invest Dermatol. 2007; 127(6):1358-66.
24. Mao R, et al. Inflamm Bowel Dis. 2019; 25(3):421-6.
25. Xu C, et al. Sci Rep. 2017; 7(1):16351
26. Koon HW, et al. Gastroenterology. 2011; 141(5):1852-63 el-3.
27. Strong S A, et al. Gastroenterology. 1998; 114(6):1244-56.
28. Sideri A, et al. Cell Mol Gastroenterol Hepatol. 2015; 1(4):420-32.
29. Hoang-Yen Tran D, et al. Int J Obes (Lond). 2016. 10.1038/ijo.2016.90
30. D'Haens G R, et al. Gastroenterology. 1998; 114(2):262-7.
31. Schauber J, et al. Eur J Gastroenterol Hepatol. 2006; 18(6):615-21.
32. Dai J, Liu W Z, et al. Scand J Gastroenterol. 2007; 42(12):1440-4.
33. Paczesny S, et al. Sci Transl Med. 2010; 2(13):13ra2
34. Erhayiem B, et al. Clin Gastroenterol Hepatol. 2011; 9(8):684-7 el.
35. Buning C, et al. Inflamm Bowel Dis. 2015; 21(11):2590-7.
36. Zhang H, et al. Front Immunol. 2019; 10:1464
37. Isakov O, et al. Inflamm Bowel Dis. 2017; 23(9):1516-23.
38. Waljee A K, et al. Inflamm Bowel Dis. 2017; 24(1):45-53.
39. Danese S, et al. Gastroenterology. 2018; 155(1):76-87.
40. Rieder F, et al. Aliment Pharmacol Ther. 2018; 48(3):347-57.
41. Wehkamp J, et al. Eur J Gastroenterol Hepatol. 2002; 14(7):745-52.
42. Wehkamp J, et al. Curr Opin Gastroenterol. 2007; 23(1):32-8.
43. Kim J M. Intest Res. 2014; 12(1):20-33.
44. Muthas D, et al. Scand J Gastroenterol. 2017; 52(2):125-35.
45. Kuno Y, et al. J Gastroenterol. 2002; 37 Suppl 14:22-32.
46. Gallo A, et al. PLoS One. 2012; 7(3):e30679
47. Gregory P A, et al. Nat Cell Biol. 2008; 10(5):593-601. 10.1038/ncb1722 .

### Example 2

### Elafin Therapy Reverses Intestinal Fibrosis by Modulating Protease-Activated Receptor 2, miR205-5p, and Zinc Finger E-Box Binding Homeobox 1 in Mice

Some Crohn's disease (CD) patients develop intestinal fibrosis. Elafin is a human protease inhibitor and antimicrobial peptide. Elafin-overexpressing bacteria are useful in ameliorating colitis in mice. We hypothesize that elafin is effective against intestinal fibrosis. This Example demonstrates the efficacy of elafin against intestinal fibrosis and elucidating its mechanism of action. Human colonic tissues, CD patient-derived primary peripheral blood mononuclear cells, colonic fibroblasts, and three mouse models of intestinal fibrosis were included. Elafin (0.1-10 micrograms/ml) significantly reduced collagen mRNA expression, increased miR205-5p expression, and decreased Zinc finger E-box-binding homeobox 1 (ZEB1) mRNA expression in transforming growth factor-beta 1 (TGF-β1)-treated colonic fibroblasts. Systemic lentiviral elafin overexpression reversed preexisting ileal fibrosis in SAMP1/YitFc mice and cecal fibrosis in Salmonella-infected mice. Intracolonic elafin overexpression also inhibited colonic fibrosis in trinitrobenzene sulfonic acid (TNBS)-treated mice. Elafin increased miR205-5p expression and decreased ZEB1 expression in human colonic fibroblasts and intestines of three intestinal fibrosis models. Elafin inhibited ERK1/2 phosphorylation via PAR2. Protease-activated receptor 2 (PAR2) agonist, inhibition of miR205-5p, and overexpression of ZEB1 reversed the elafin's anti-fibrogenic effects in human colonic fibroblasts, colons of TNBS-treated mice, and ceca of Salmonella-infected mice. Oral Elafin-Eudragit FS3OD formulation significantly inhibited TNBS-mediated colonic fibrosis in mice. This Example thus shows that elafin suppresses collagen synthesis in intestinal fibroblasts via PAR2 inhibition, increased miR205-5p expression, and decreased ZEB1 expression, leading to the inhibition of collagen synthesis and reversal of intestinal fibrosis. Modified elafin provides a therapeutic tool for treating intestinal fibrosis.

Intestinal stricture is a debilitating complication of inflammatory bowel disease (IBD) [1]. Anti-tumor necrosis factor-alpha (anti-TNFa) neutralizing antibodies fail to reverse intestinal strictures in CD patients [2]. Surgical resection may be used to treat intestinal fibrosis. However, surgery may affect the quality of life of the patients adversely. Thus, new therapeutic approaches to intestinal fibrosis are needed.

Antimicrobial peptides have anti-inflammatory and anti-fibrogenic effects in colitis models [3-6]. Elafin is an elastase-specific protease inhibitor and antimicrobial peptide [7, 8]. Elafin mRNA expression is reduced in the peripheral blood leukocytes of IBD patients [9, 10]. Colonic elafin mRNA expression is increased in ulcerative colitis (UC) patients [11, 12], while there is no increase of colonic elafin mRNA and protein expression in CD patients [13]. Interestingly, the colonic elafin expression is almost abolished in stricturing CD patients [12].

We hypothesize that elafin may be useful for treating intestinal fibrosis. Adenoviral delivery of elafin ameliorated chemically induced colitis in mice [14]. Oral administration of elafin-expressing Lactococcus inhibited colitis and gluten-related disorders in mice [15, 16]. However, lactic acid bacteria may cause adverse effects among the immunosuppressed population [17, 18]. Intravenous elafin injection (200 mg/person or >3000-fold increase of plasma concentration) does not cause any adverse effects in humans [19], but it is invasive and inconvenient.

This study discovered novel targets in intestinal strictures and examined the anti-fibrogenic mechanism of elafin using stricturing CD patient-derived primary fibroblasts and three CD-relevant mouse models of intestinal , including SAMP1/YitFc mice carrying CD-like microbial signature [20, 21], Salmonella-infected mice with cecal Th1/Th17 cytokine activation [22], and well-established chronic TNBS colitis [6, 23, 24]. Our group utilized advanced pharmaceutical technologies to develop an orally active elafin formulation.

Materials and Methods

Human Colonic Tissue Samples

Human colonic tissue samples of non-IBD, UC, and CD patients were collected from the Cedars-Sinai Medical Center [20]. The study was approved by the Cedars-Sinai Institutional Review Board (IRBs 3358 and 23705) and UCLA Institutional Review Board (IRB-11-001527). All samples were collected during the indicated diagnostic procedure from 2010 to 2014 prospectively. Informed consent was obtained from all subjects by the Cedars-Sinai Medical Center. Separate informed consent was waived by UCLA IRB. All methods were carried out in accordance with relevant guidelines and regulations.

Inclusion criteria: IBD and intestinal strictures were diagnosed by gastroenterologists. Intestinal strictures were defined by prestenotic dilation, luminal narrowing, and increased wall thickness in magnetic resonance enterography or endoscopy observations. Exclusion criteria: Pregnant women, prisoners, or minors under age 18 were not included. Additionally, patients with concurrent acute infection (CMV, C. difficile, and tuberculosis) and malignant conditions were excluded.

Colonic tissue samples of IBD patients were collected during surgical resection of diseased tissues. Colonic tissue samples of control group patients were collected during surgical removal of colonic tumors and adjacent normal tissues. The surgeons and pathologists confirmed the presence of intestinal stricture or colon cancer. The colonic tissues with normal histology were used as non-IBD control tissue samples. Baseline characteristics are shown in Table 1.

TABLE 1
Baseline characteristics of human colonic tissue samples
(upper panel) and human serum samples (lower panel).

| | | | CD without | CD without |
|---|---|---|---|---|
| Colonic tissues | non IBD | UC | stricture | stricture |
| Elafin mRNA | 5.7 ± 1.89 | 11.8 ± 2.67 | 5.4 ± 1.0 | 2.9 ± 2.3 |
| Expression (fold) | | | | |
| Age at Collection | 62 ± 2.2 | 43 ± 2.1 | 45 ± 3.6 | 36 ± 6.1 |
| (Mean ± SEM) | | | | |
| Gender (% Male) | 73 | 55 | 62 | 50 |
| Histology Score | 2.6 ± 0.3 | 7.5 ± 0.4 | 8.8 ± 0.7 | 8.6 ± 1.2 |
| (Mean ± SEM) | | | | |
| Simple colitis activity | N/A | 6.8 ± 0.6 | N/A | N/A |
| score | | | | |
| HBI | N/A | N/A | 7.4 ± 1.2 | 5.0 ± 0.5 |
| % of biologics | 0 | 24 | 50 | 40 |
| % of 6MP or steroid | 0 | 51 | 66 | 33 |
| Duration of disease | 26 ± 3 | 12 ± 2 | 8 ± 3 | 18 ± 3 |
| (years) | | | | |
| n | 40 | 52 | 28 | 35 |
| | | | CD without | CD without |
| Serum samples | Normal | UC | stricture | stricture |
| Serum elafin levels | 7939 ± 791 | 12987 ± 1124 | 7042 ± 520 | 11263 ± 1817 |
| (pg/ml) | | | | |
| Age at Collection | 46 ± 12 | 40 ± 2 | 34 ± 2 | 40 ± 3 |
| (Mean ± SEM) | | | | |
| Gender (% Male) | 42 | 35 | 40 | 60 |
| Harvey Bradshaw | N/A | N/A | 3.7 ± 0.7 | 65 ± 1.2 |
| Index | | | | |
| Partial Mayo Score | N/A | 2.1 ± 0.4 | N/A | N/A |
| % of biologics | N/A | 13 | 40 | 46 |
| % of 6MP or steroid | N/A | 12 | 33 | 42 |
| Duration of disease | N/A | 6 ± 2 | 11 ± 1.4 | 11 ± 2.3 |
| (years) | | | | |
| n | 40 | 52 | 28 | 35 |

Human Serum Samples

Serum samples of normal, UC, and CD patients were collected from UCLA from 2012 to 2015 prospectively. The physicians requested the medically indicated blood collection.

This study was approved by the UCLA Institutional Review Board (IRB 12-001499). Separate informed consent was waived by UCLA IRB because UCLA Pathology obtained written informed consent from all subjects. Baseline characteristics are shown in Table 1.

Inclusion criteria: IBD and intestinal strictures were identified by gastroenterologists. Exclusion criteria: IBD patients with concurrent acute infection (CMV, C. difficile, and tuberculosis) and malignancy were excluded. Pregnant women, prisoners, or minors under age 18 were excluded.

Human Colonic Fibroblasts

Stricturing CD patient-derived primary colonic fibroblasts (CD-HIFs) were obtained from Cleveland Clinic via material transfer agreement (MTA2020-00000154) and under

Cleveland Clinic IRB directives (IRB #17-1167). Primary non-IBD patient-derived colonic fibroblasts (H6231) were purchased from Cell Biologics. All primary colonic fibroblasts were cultured in fibroblast medium (M2267, Cell Biologics). Human colonic CCD-18Co fibroblasts (ATCC) were cultured in minimal essential medium Eagle's medium (MEM) containing 10% fetal bovine serum and 1% penicillin-streptomycin 6, 20. All fibroblasts were grown to 80% confluence and then switched to serum-free media overnight for experiments.

For activation of protease-activated receptors (PARs), fibroblasts were pretreated with 0.8% DMSO, 10 μM PAR1 agonist TRAP-6 (HY-P0078, MCE), 10 μM PAR2 agonist SLIGKV-NH2 (Protease-Activated Receptor-2, amide, HY-P0283, MCE), or 0.4 μg/ml PAR2-activating protease recombinant cathepsin S (1183-CY-010, R&D Systems) for 30 minutes. For miR205-5p activation, fibroblasts were transfected with (30 picomoles/well) either control (YM00479902) or miR205-5p miRCURY LNA mimic (YM00472340) from Qiagen via Lipofectamine 3000 transfection reagent (L3000001, ThermoFisher) in Opti-MEM (#31985062, ThermoFisher) overnight. For miR205-5p inhibition, fibroblasts were pretreated with 50nM either control (Y100199006) or miR205-5p power inhibitors (Y104101508-DDA) from Qiagen overnight. For overexpression of zinc finger E-box homeobox 1 (ZEB1), fibroblasts were infected with 104 infectious units/plate either control (PS100064V) or human ZEB1-overexpressing lentivirus (RC217704L1V) from Origene, Inc. overnight.

The fibroblasts were pretreated with either 0.1% TFA (vehicle), 10 ng/ml transforming growth factor-beta 1 (TGF-p1), or 100 μg/ml serum exosomes from stricturing CD (CDSE) patients for two hours to induce fibrogenesis [12], followed by incubation with elafin (AS-61641, Anaspec) for 2-24 hours. Serum exosomes from CD patients were used to mimic the chemical environment of CD [12]. At the end of the experiments, the cells were lysed with either RLT buffer for RNA experiments or radioimmunoprecipitation assay (RIPA) buffer for ProCOL1A1 (DY6220-05, R&D Systems), COL1A2 (MBS2701496, MyBioSource), ZEB1 (MBS774017, MyBioSource), or phosphorylated ERK1/2 (DYC1018B, R&D Systems) ELISAs.

CD Patient-Derived Primary Peripheral Blood Mononuclear Cells (CD PBMCs)

CD PBMCs (#70052, STEMCELL Technologies) were cultured in RPMI1640 media containing 10% exosome-depleted fetal bovine serum (A2720803, ThermoFisher) and 1% penicillin-streptomycin. Some groups were conditioned with 100 μg/ml of human serum exosomes from either CDSE or non-stricturing CD (CDNSE) patients to mimic the CD chemical environment for two hours [12], followed by addition with either 0.1% TFA or 1 μg/ml elafin. After six hours of incubation, the cells were removed by centrifugation at 10,000 g for five minutes at 4 oC. The cell-free supernatants were collected for multiplex assay.

Animal Experiments

All animal studies were approved by UCLA Institutional Animal Research Committee (#2007-116). All methods were compliant with the ARRIVE guidelines. Mice were randomized and assigned to cages by animal facility staff in a blind manner and housed in the UCLA animal facility under standard environmental conditions. All interventions were performed during the light cycle. At the end of the experiments, serum samples were collected for mouse cytokine 23-plex multiplex assay (#M60009RDPD, Bio-Rad).

Trinitrobenzene Sulfonic Acid (TNBS)-Associated Colonic Fibrosis in Mice

Eight-week-old male and female CD-1 mice (#022, Charles River Laboratories) were injected with 50pL TNBS solution (to induce colonic fibrosis) or 30% ethanol (vehicle) via enema weekly for five weeks [6, 23, 25]. After the last TNBS injection, the mice were held for two additional weeks to develop colonic fibrosis. Some of the mice were injected with 5 μg/mouse of either control (PS100001) or elafin-expressing construct (RC203136) from

Origene via InvivoJetPEI transfection reagent (201-10G, Polyplus) intracolonically on the 9th day after the last TNBS injection or five days before the end of experiment [26].

Some of the mice received either a single intraperitoneal injection of 10 mg/kg anti-tumor necrosis factor-alpha (anti-TNFα) neutralizing antibody (6E0058, BioXCell) or oral antibiotic mixture in drinking water ad libitum from the 9th day after the last TNBS injection to the endpoint of the experiment. The antibiotics mixture contains kanamycin (0.4 mg/mL), gentamicin (0.035 mg/mL), colistin (850 U/mL), metronidazole (0.215 mg/mL), and vancomycin (0.045 mg/mL), which was routinely used by our laboratory for suppressing intestinal microbiota [4, 27].

For PAR1/2 activation, some of the mice were injected with DMSO (50pL/mouse), 5 mg/kg PAR1 agonist TRAP-6, or 5 mg/kg PAR2 agonist SLIGKV-NH2 via enema on the 9th, 11th, and 13th days after the last TNBS injection.

For miRNA inhibition, some of the mice were injected with 5 mg/kg either control inhibitor or miR205-5p power inhibitor (Qiagen) daily via enema on the 9th, 11th, and 13th days after the last TNBS injection.

For Zeb1 manipulation, some mice received a single intraperitoneal injection (10⁷ infectious units/mouse) of control lentivirus (PS100064V), Zeb1-overexpressing lentivirus (MR223095L2V), or Zeb1-shRNA lentivirus (TL513177V) from Origene, Inc. nine days after the last TNBS injection.

Dr. Xingguo Cheng produced the oral elafin-Eudragit-FS3OD formulation via a material transfer agreement (UCLA MTA2019-00000337) with the Southwest Research Institute (SWRI) in Texas. This pH-responsive formulation is insoluble in acid but dissolves in a mildly alkaline environment (i.e., pH 7-9). The mice were fed with either control-Eudragit or elafin-Eudragit in mildly acidified (pH 5) water containing 0.5% hydroxypropyl methylcellulose (HPMC) daily via oral gavage during the last five days of the experiment.

Salmonella-mediated cecal fibrosis in mice

Eight-week-old male and female 129Sv/J mice (#000691, Jackson Laboratories) were administered 20 mg streptomycin via oral gavage. Twenty-four hours later, the mice were orally infected with Salmonella typhimurium SL1344 strain 1×10⁸ colony-forming units by oral gavage to induce cecal fibrosis [22]. Some infected mice received a single intraperitoneal injection (1×10⁷ infectious units per mouse) of control lentivirus, elafin-overexpressing lentivirus, Zeb1-shRNA lentivrus, or Zeb1-overexpressing lentivirus from

Origene or miR205-5p-OFF lentivrus (mm35328) or miR205-5p-OE lentivirus (mm15214) from Applied Biological Materials on day 14. Some infected mice received oral antibiotics mixture in drinking water ad libitum from day 14 to day 21 to suppress intestinal microbiota [4, 27]. Other infected mice received 10mg/kg/day either PAR2 agonist GB110 (HY-120528A, MCE) or PAR2 inhibitor GB88 (#2300100052, Eton Bioscience) via oral gavage from day 14 to day 21. Cecal tissues were collected for analysis on day 21.

Spontaneous Ileal Fibrosis in SAMP1/YitFc Mice

Eight-week-old male and female SAMP1/YitFc mice (#009355) and normal AKR strain control mice (#000648) were purchased from Jackson Laboratories. SAMP1/YitFc mice developed spontaneous ileal inflammation and fibrosis at 40 weeks of age [20, 28].

Some groups were treated with antibiotics mixture in drinking water ad libitum from 10 to 42 weeks of age. Others received a single intraperitoneal injection (1×10⁷ infectious units/mouse) of either control lentivirus or elafin-expressing lentivirus (RC203136L1V, Origene) at 40-weeks of age. Ileal tissues were collected for analysis two weeks after the lentiviral injection.

Microbiome Analysis

(Genomic DNA Extraction and Amplification) Genomic DNA was extracted from the ileal samples using ZymoBIOMICS DNA Miniprep Kit (Zymo Research, Irvine, CA). The hypervariable regions V3-V5 of the 16S rRNA gene were amplified from purified genomic DNA using universal primers 341F and 926R. PCR amplification was performed according to the protocol developed by the Human Microbiome Project [29]. Illumina indices were added to the purified amplicons based on the Illumina MiSeq specifications (Illumina, Inc., San Diego, CA). 16S rRNA amplicon libraries were purified, quantified using qPCR, and pooled for sequencing on the Illumina MiSeq platform (Illumina, Inc., San Diego, CA).

(Microbial Taxonomic Composition Analysis) A data cleaning process was applied to all sequence data before analysis. Briefly, low-quality bases with a Phred quality value lower than 20 were trimmed off the read ends. The read pairs were removed if any of the two reads was trimmed to shorter than 200 bp or had 3% uncertain bases. The 16S rRNA sequences with paired reads were mapped against Greengenes (v13.5, non-redundant precalculated OTU references, 97_otus from PICRUSt) [30, 31]. The alignments were performed using Bowtie2 to search sequences with the best hits to the references [32]. Each sample's taxonomic composition was inferred at the phylum level, family level, and genus level.

(Analysis of the Microbial Community) The microbial community evenness and richness were measured by alpha diversity, and the similarity between individual microbial communities was measured by beta diversity. Alpha diversity (Shannon index), beta diversity (weighted UniFrac), and Principal Coordinate Analysis (PCoA) were calculated or performed using QIIME [33].

Histological Evaluation of Intestinal Injury and Fibrosis

H&E and Masson Trichrome (MT) staining were performed as described previously [6, 24]. Microphotographs were recorded at multiple locations and scored by two investigators blindly [6, 24]. Intestinal injuries were scored (0-12) [34]. The severity of intestinal fibrosis was assessed by MT staining and scored (0-3) [35]. ZEB1 immunohistochemistry was performed using an anti-ZEB1 antibody (HPA027524, Sigma).

Quantitative Real-Time RT-PCR

Total RNA was isolated by an RNeasy kit (#74104, Qiagen) and reverse transcribed into cDNA by a high-capacity cDNA reverse transcription kit (#4368813, ThermoFisher). PCR reactions were run with Taq Universal SYBR Green Supermix (1725120, Bio-Rad) and TaqMan real-time PCR assays (ThermoFisher) in a Bio-Rad CFX384 system [36]. For the determination of miRNA expression, RNA was converted to cDNA using miRCURY LNA RT kit. PCR reactions were run with miRCURY LNA SYBR Green PCR kit and miRCURY LNA PCR assays (Qiagen). Catalog numbers of PCR reagents are shown in Table 2. Relative mRNA/miRNA quantification was performed by comparing test groups and control groups after normalization with endogenous control genes. The fold changes are expressed as 2^{ΔΔCt}. Fold-change values greater than one indicates a positive or an up-regulation, and the fold-regulation is equal to the fold-change. Fold-change values less than one indicate a negative or down-regulation, and the fold-regulation is the negative inverse of the fold-change.

TABLE 2
Reagents

| | Catalog number |
|---|---|
| Human primers | Source: Thermo Fisher |
| COL1A2 | (Hs01028956_m1) |
| COL3A1 | (Hs00943809_m1) |
| TGF-b1 | (Hs00998133_m1) |
| ACTA2 | (Hs00426835_g1) |
| Elafin or PI3 | (Hs00160066_m1) |
| ZEB1 | (Hs01566408_m1) |
| 18S (endogenous control) | (Hs99999901_s1) |
| Mouse primers | Source: Thermo Fisher |
| Col1a2 | (Mm00483888_m1) |
| Col3a1 | (Mm00802300_m1) |
| Tgf-b1 | (Mm01178820_m1) |
| Zeb1 | (Mm00495564_m1) |
| Vim | (Mm01333430_m1) |
| Acta2 | (Mm00725415_s1) |
| Tnf | (Mm00443258_m1) |
| Emr1 | (Mm00802529_m1) |
| Gapdh (endogenous control) | (Mm99999915_g1) |
| PCR reagents | Source: Thermo Fisher |
| high-capacity cDNA RT kit | 4368813 |
| Fast Universal PCR master mix | 4332042 |
| miRNA reagents | Source: Qiagen |
| miR205-5p miRCURY LNA PCR assay | YP00204487 |
| RNU1A1 miRCURY LNA PCR assay | YP00203909 |
| (endogenous control) | |
| miRCURY LNA RT kit | 339340 |
| miRCURY LNA SYBR Green PCR kit | 339346 |

Calculation of Overall Disease Activities

Overall disease activity (ODA) was calculated by % change of histology and fibrosis scores and colonic mRNA expression of fibrosis- and inflammation-related genes [6, 22-24]. Col1a2 is the typical fibrosis signature among models [22, 23, 37, 38]. Col3a1 was not 5 included in the Salmonella model because Salmonella infection did not affect cecal Col3a1 mRNA expression [22]. The mRNA expression lower than the control group in negative values were assigned zero number, indicating complete inhibition.

Power Analysis

Animal study: At least four mice per group were required to achieve a statistically significant difference of histology score between the TNBS group and TNBS+anti-TNFα group with standard deviation=0.9, alpha=0.5, and power=0.8. The present study satisfied this requirement.

The colonic tissue cohort consisting of 40 non-IBD, 52 UC, and 43 CD patients provided adequate power to detect the difference of colonic elafin mRNA expression [12]. We did not perform power analysis for cell culture experiments but followed the common practice of performing in vitro experiments three times independently.

Statistical Analysis

Results were expressed as mean+/−SEM. Unpaired Student's t-tests were used for two-group comparisons of continuous data (GraphPad) online. One-way ANOVAs with Tukey Honestly Significant Difference post-hoc tests were used for multiple-group comparisons (Statpages) online. The p values of statistical significance are shown in each figure.

Results

Elafin inhibited collagen expression in human colonic fibroblasts directly.

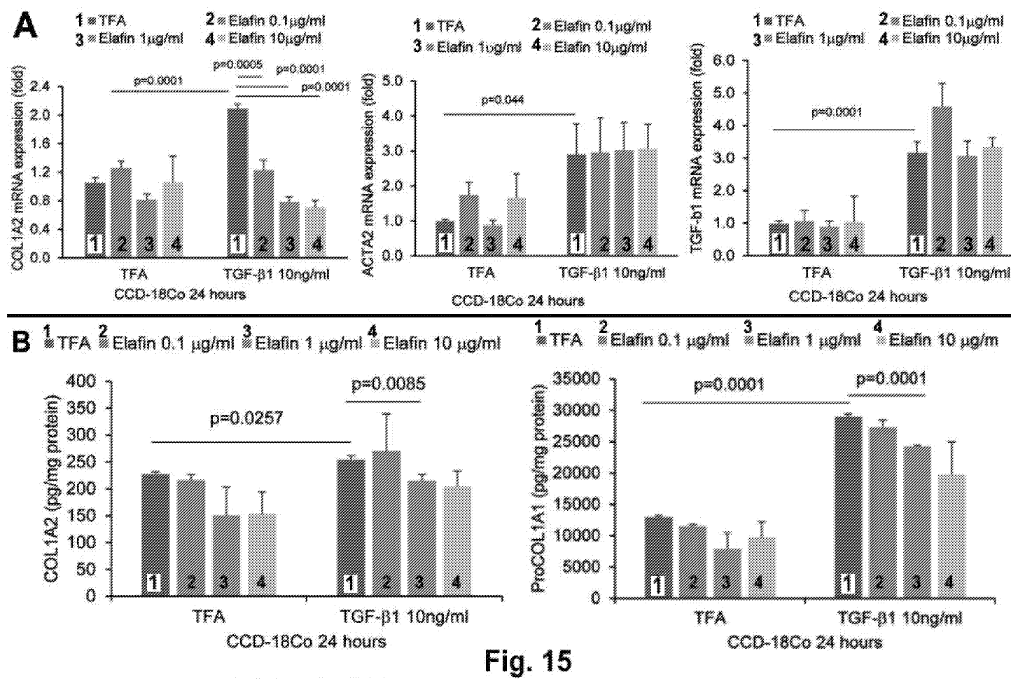

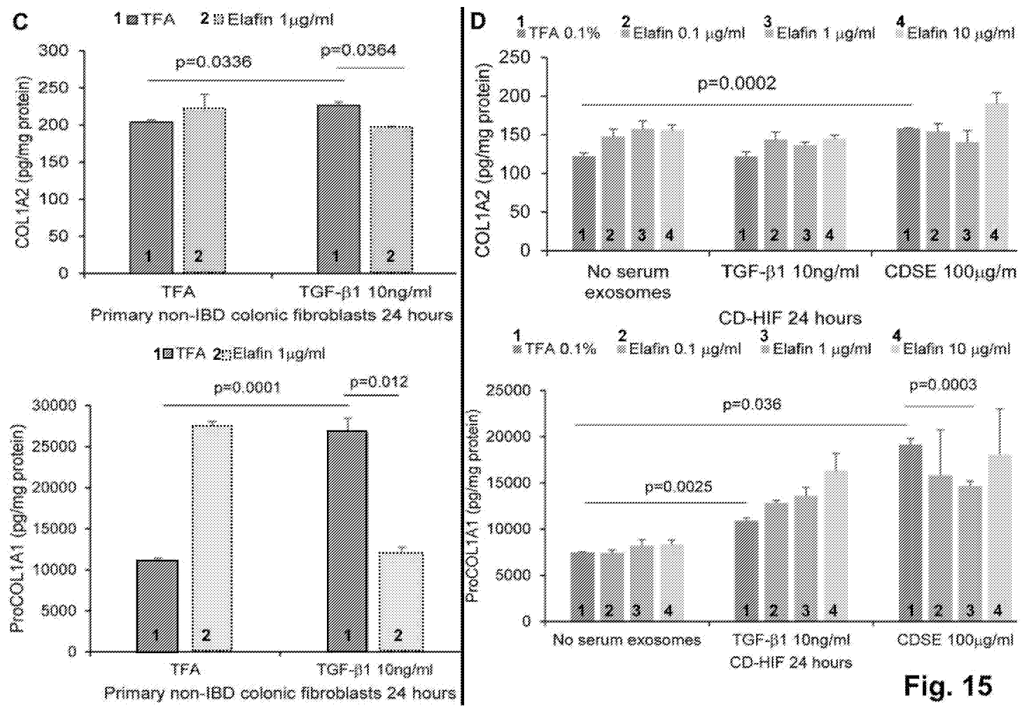

Collagens (COL1A1 and COL1A2) are stricture-dependent genes in the intestine of CD patients [2, 12]. To determine whether elafin affects fibrogenesis directly, we first induced collagen COL1A2 mRNA expression in human colonic CCD-18Co fibroblasts with TGF-β1 [6, 24]. Elafin reversed TGF-β1-induced COL1A2, but not actin alpha 2 (ACTA2) and TGF-β1 mRNA expression in CCD-18Co fibroblasts (FIG. 15A). Similarly, elafin 30 reversed TGF-β1 induced COL1A2 and ProCOL1A1 protein expression in CCD-18Co and primary non-IBD patient-derived colonic fibroblasts (FIG. 15B-C). Elafin (1 μg/ml) inhibited ProCOL1A1, but not COL1A2 protein expression in CDSE-treated primary stricturing CD patient-derived colonic fibroblasts (FIG. 15D) [12]. In general, elafin inhibits collagen synthesis in activated colonic fibroblasts.

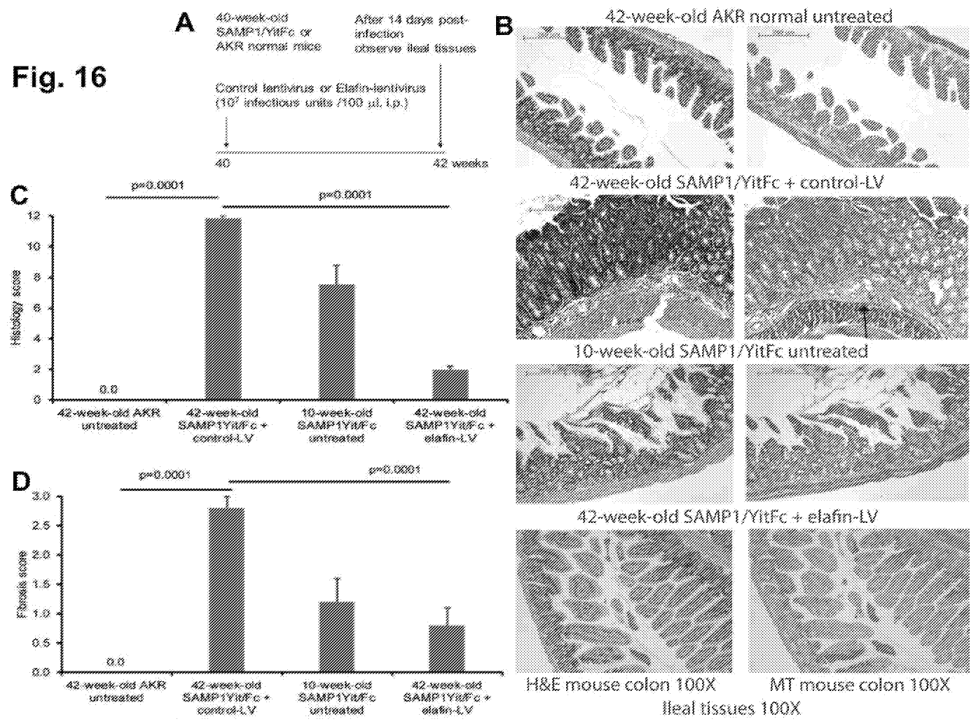

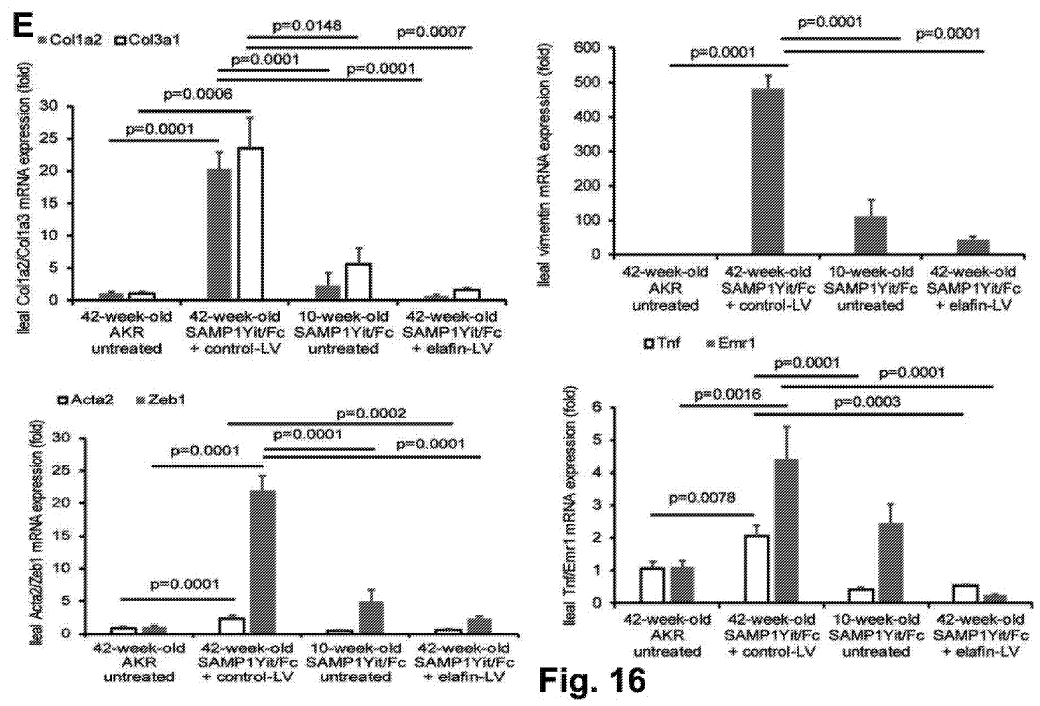

Systemic elafin overexpression inhibited ileal fibrosis in 42-week-old SAMP1/YitFc mice.
We used a spontaneous CD-like ileitis mouse model to evaluate whether elafin can reverse preexisting ileal fibrosis [20, 28]. Systemic elafin overexpression was achieved by injecting elafin-overexpressing lentivirus into the 40-week-old SAMP1/YitFc mice (FIG. 16A). Compared to 42-week-old normal AKR mice, the young 10-week-old SAMP1/YitFc mice developed spontaneous ileitis (FIG. 16B-D). Systemic elafin overexpression ameliorated the ileal mucosal disruption and collagen deposition (FIG. 16B-D). The increased ileal collagen (Col1a2, Col3a1), fibroblast (Vim, Acta2), tumor necrosis factor (Tnt), and macrophage (Emr1) mRNA expression was reversed by elafin overexpression (FIG. 16E), suggesting anti-fibrogenic and anti-inflammatory effects.

Systemic elafin overexpression inhibited cecal fibrosis in Salmonella-infected mice.

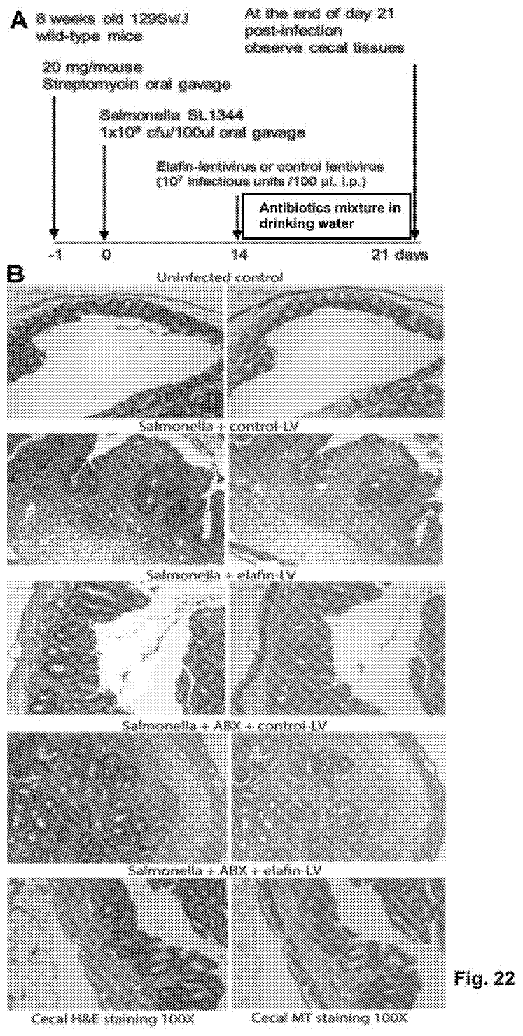

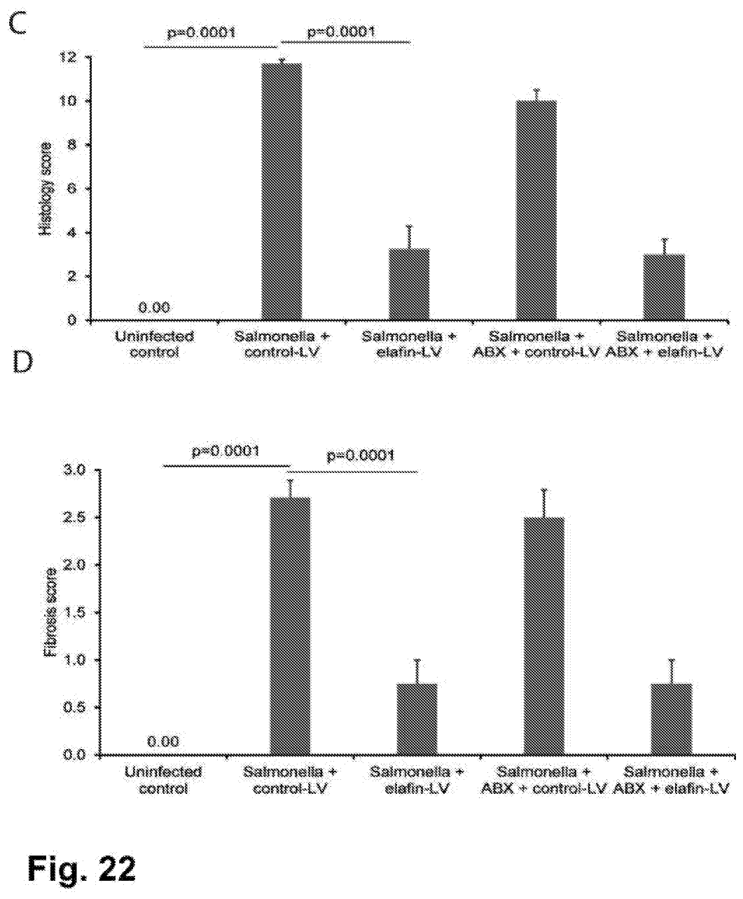

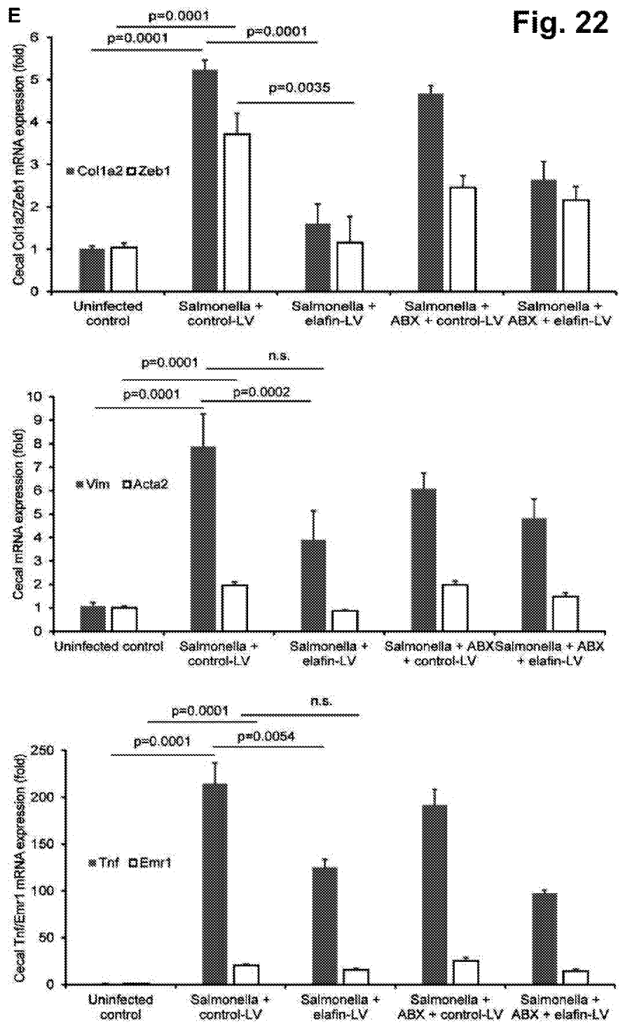

Salmonella model developed cecitis, followed by cecal fibrosis from day 14 to day 21 (FIG. 22A) [35, 39]. Lentiviral elafin overexpression ameliorated cecal epithelial disruption, immune cell infiltration, and collagen deposition in the infected mice, as reflected by low histology and fibrosis scores (FIG. 22B-D). Elafin overexpression also significantly reduced cecal Col1a2, Vim, and Tnf mRNA expression in the infected mice (FIG. 22E).

Elafin increased miR205-5p and inhibited ZEB1 expression in fibroblasts.

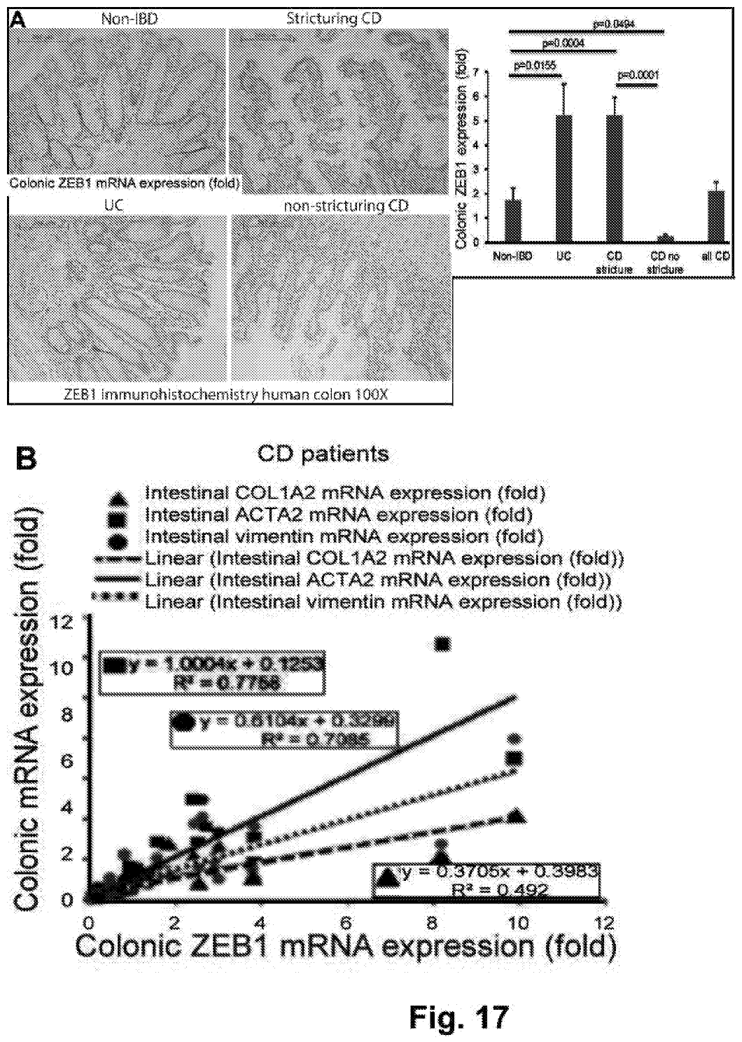

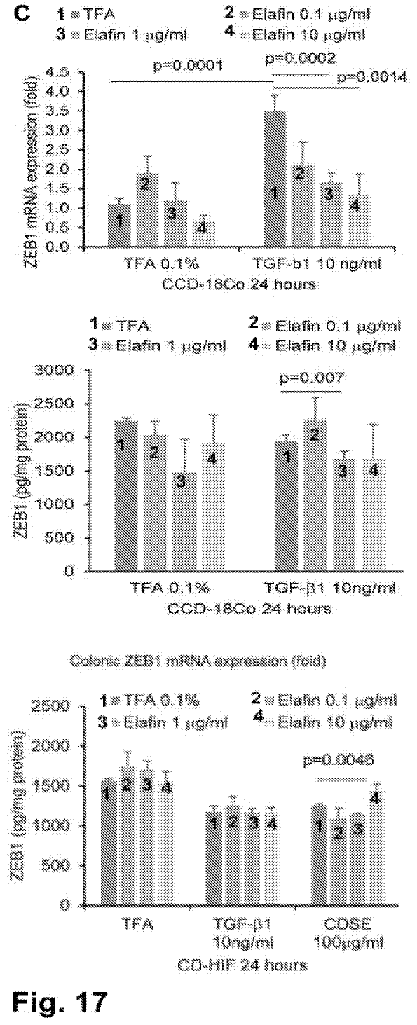

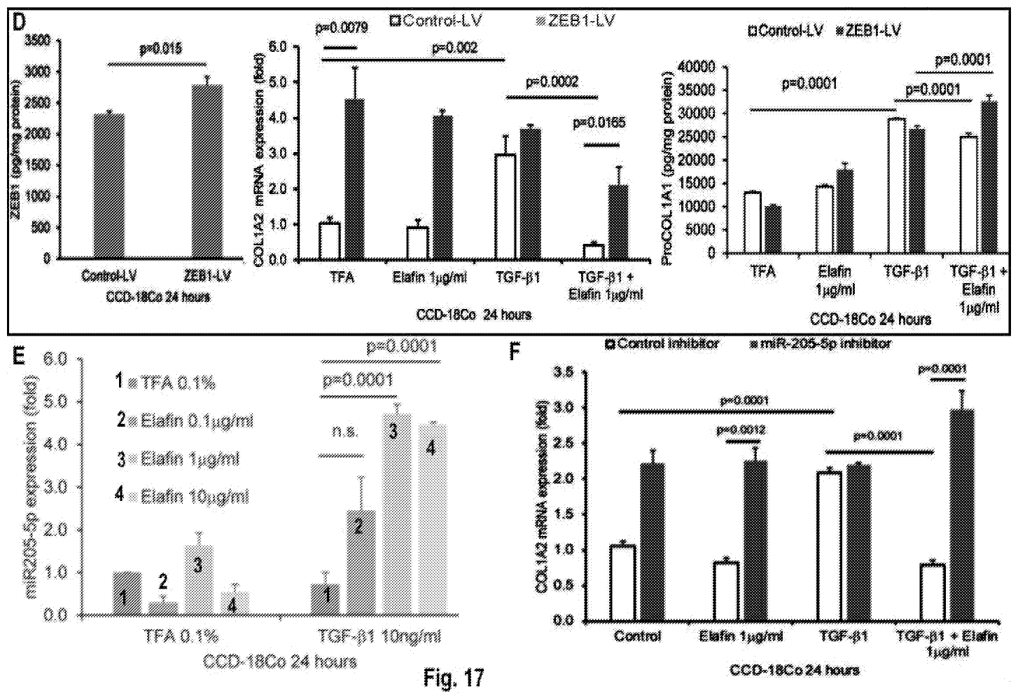

Our recent RNA-sequencing experiment revealed increased colonic ZEB1 mRNA expression in stricturing CD patients [12]. Stricturing CD patients had significantly higher colonic ZEB1 mRNA and protein expression than non-stricturing CD patients and control patients (FIG. 17A). Colonic ZEB1 mRNA expression was positively correlated with COL1A2, ACTA2, and vimentin mRNA expression in CD patients (FIG. 17B).

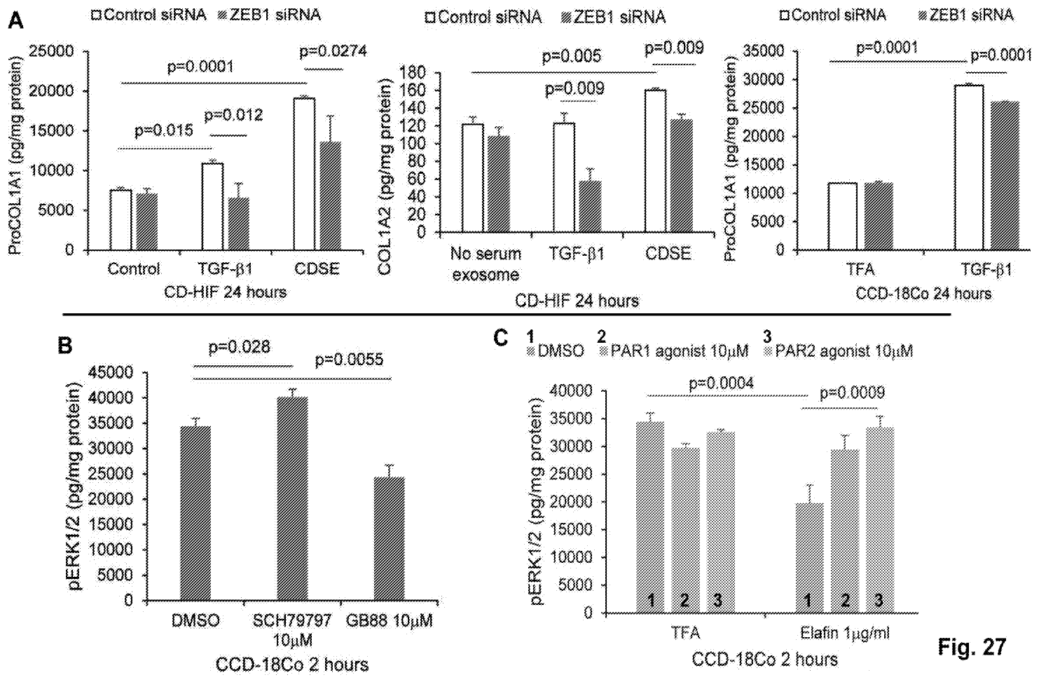

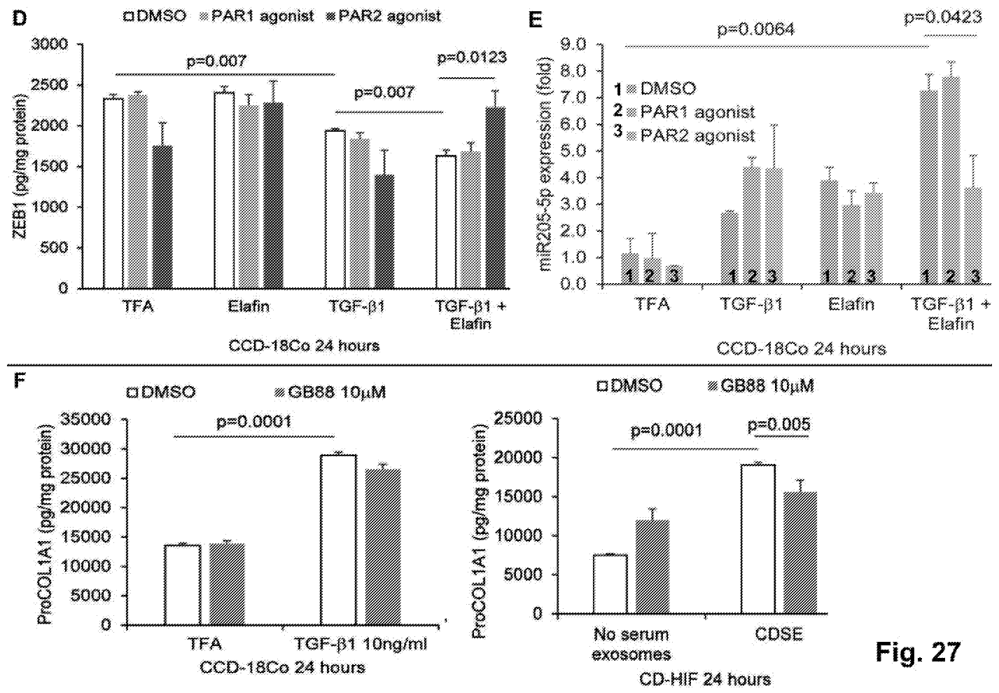

Elafin diminished ZEB1 mRNA and protein expression in TGF-β1-/CD serum-exosome-treated human colonic fibroblasts (FIG. 17C). ZEB1 knockdown significantly reduced collagen expression in fibroblasts (FIG. 27A). Lentiviral ZEB1 overexpression increased collagen protein expression and reversed elafin-mediated inhibition of collagen expression in the TGF-β1-treated CCD-18Co fibroblasts (FIG. 17D).

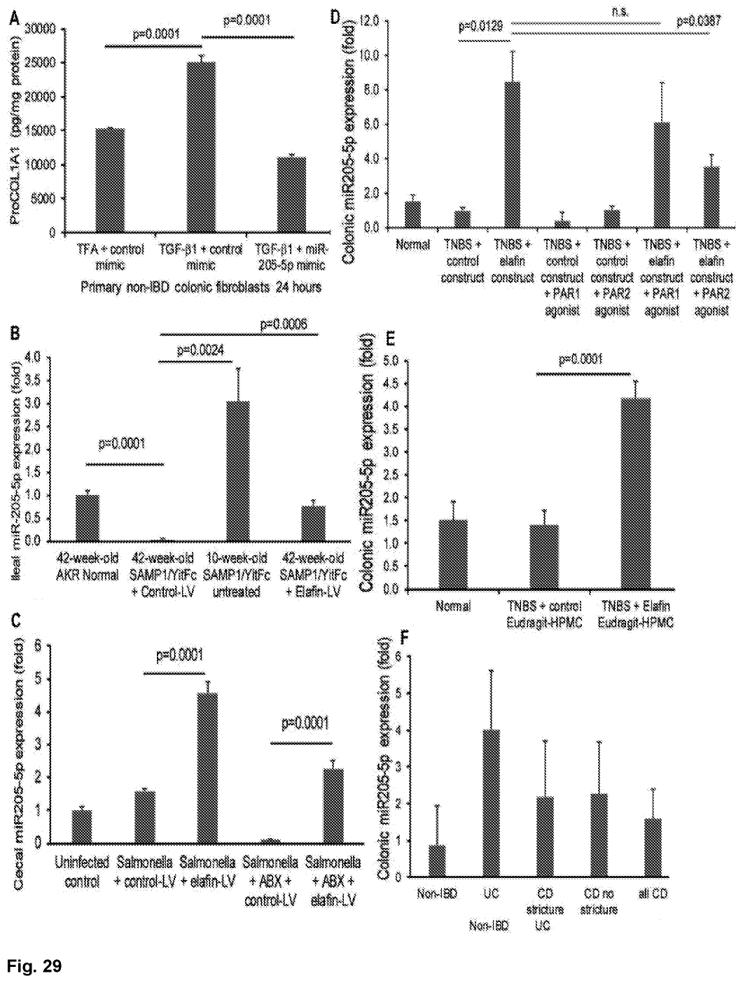

Target prediction databases (TargetScanHuman, miRWalk, and miRDB) and multiple studies suggested that miR205 targets ZEB1 expression and inhibits epithelial-mesenchymal transition (EMT) [40-42]. Stricturing CD patients have reduced serum exosomal miR205-5p expression [12]. Interestingly, elafin significantly increased miR205-5p expression in the TGF-β1-treated colonic CCD-18Co fibroblasts (FIG. 17E). miR205-5p mimic abolished TGF-p1-induced ProCOL1A1 protein expression (FIG. 29A), while miR205-5p inhibitor reversed the elafin-mediated reduction of COL1A2 mRNA expression (FIG. 17F). Thus, elafin inhibited fibrogenesis by inducing anti-fibrogenic miR-205-5p and inhibiting pro-fibrogenic ZEB1 expression.

Intracolonic elafin overexpression inhibited colonic fibrosis via miR205-5p in the TNBS-treated and Salmonella-infected mice.

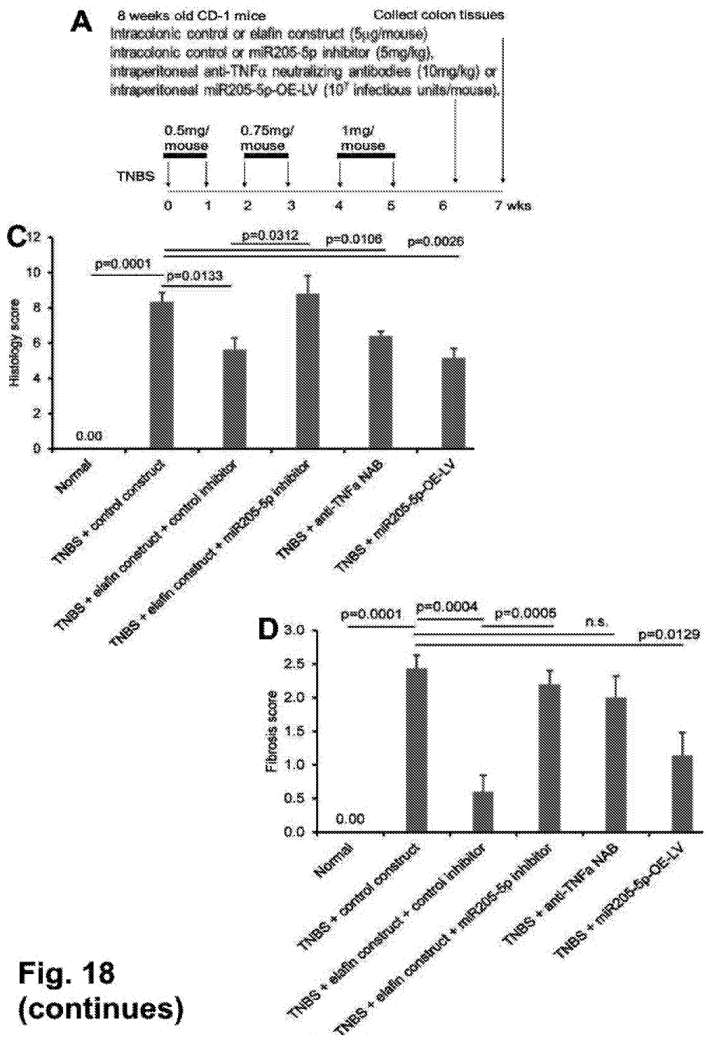

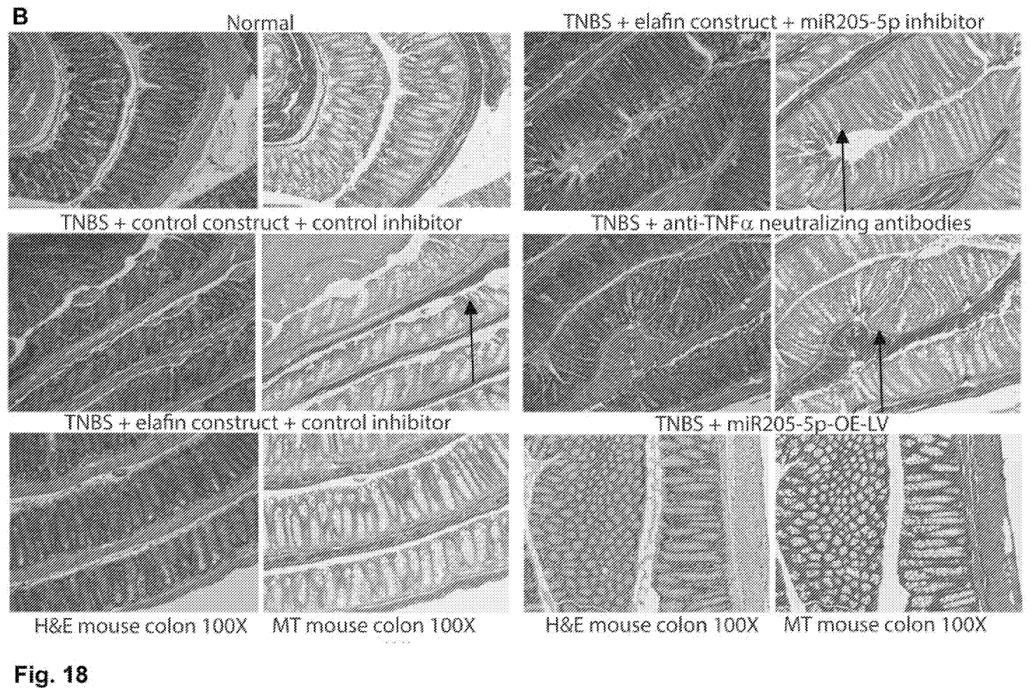

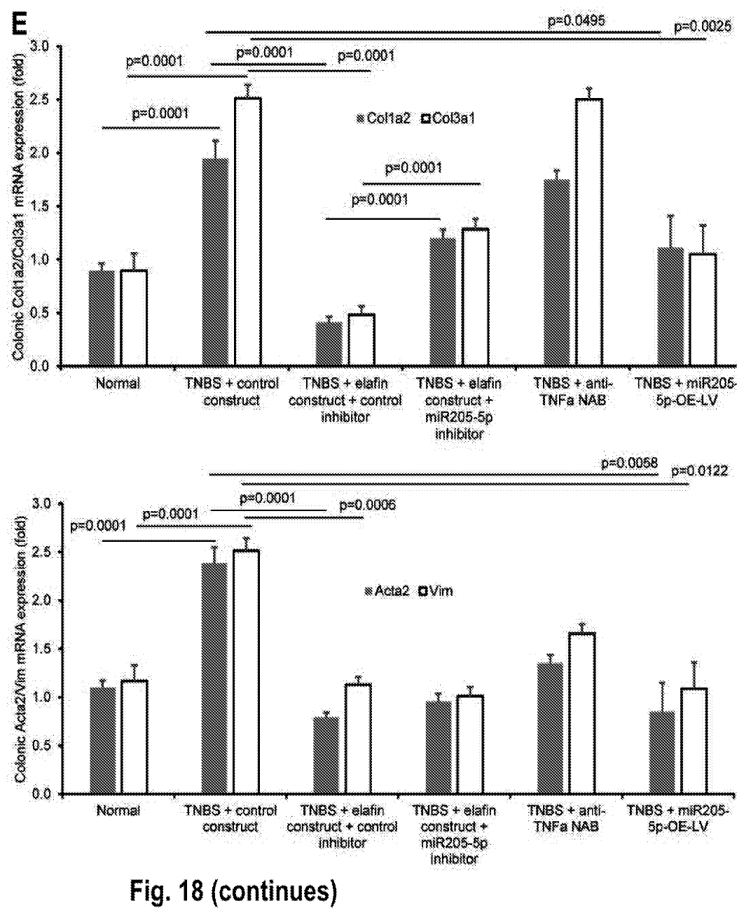

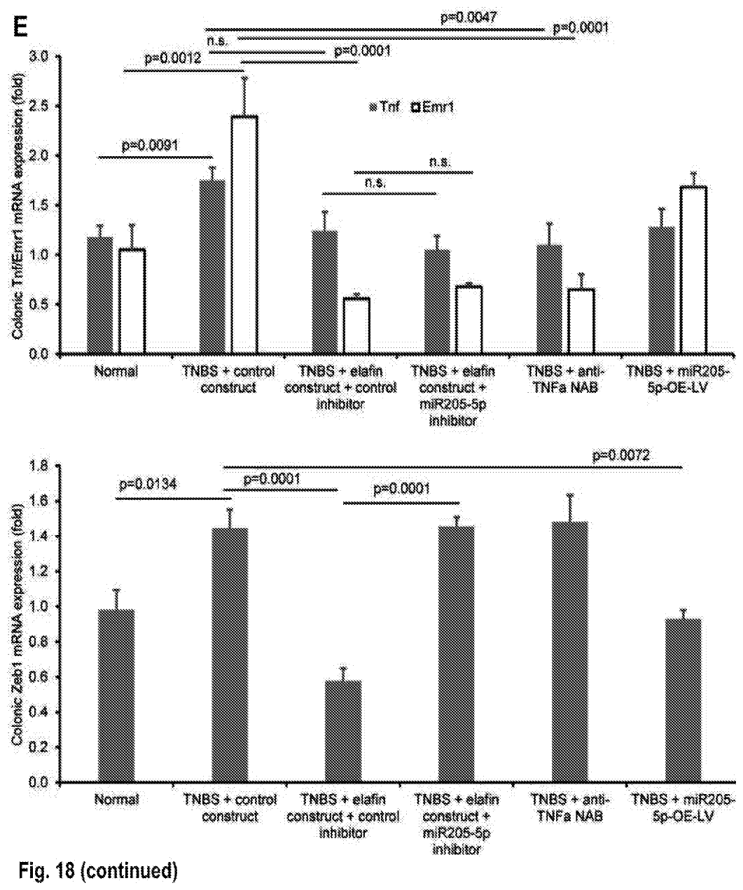

We induced colonic fibrosis with intracolonic TNBS injections [6, 24], followed by elafin-overexpressing construct and miR205-5p inhibitor transfection to determine the interactions between elafin and miR205-5p (FIG. 18A). Mice do not have the elafin gene, but the elafin-overexpressing construct expressed elafin mRNA signal in the colon (Ct value ˜35). Elafin overexpression induced ileal, cecal, and colonic miR205-5p expression in SAMP1/YitFc, Salmonella, and TNBS models, respectively (FIG. 29B-D). Intracolonic elafin overexpression also ameliorated colonic injury and fibrosis in the TNBS-treated mice (FIG. 18B-D).

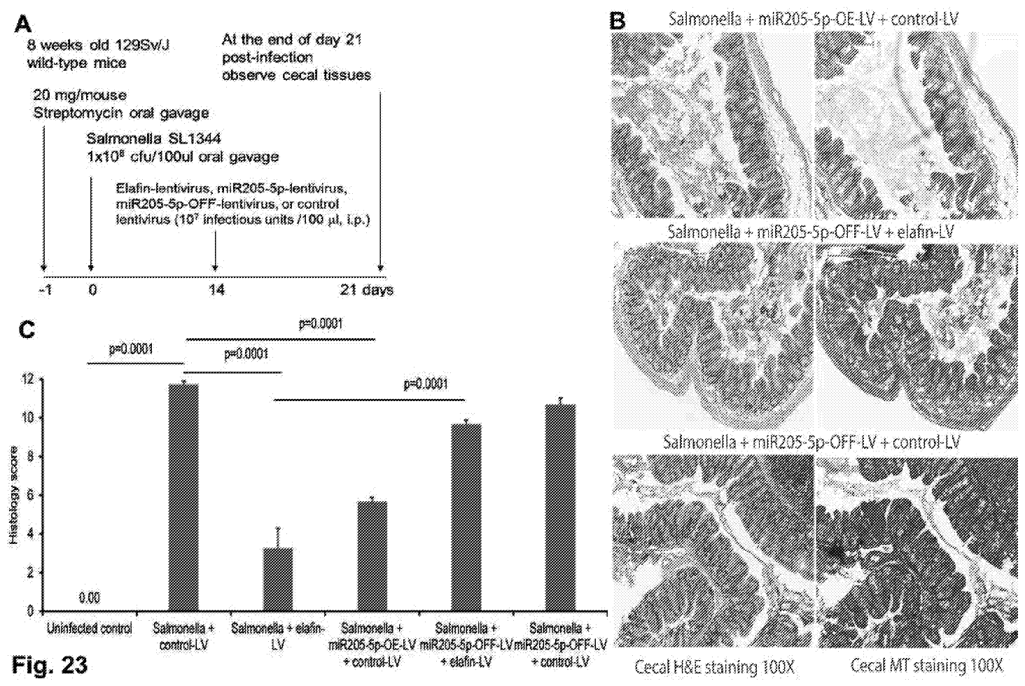

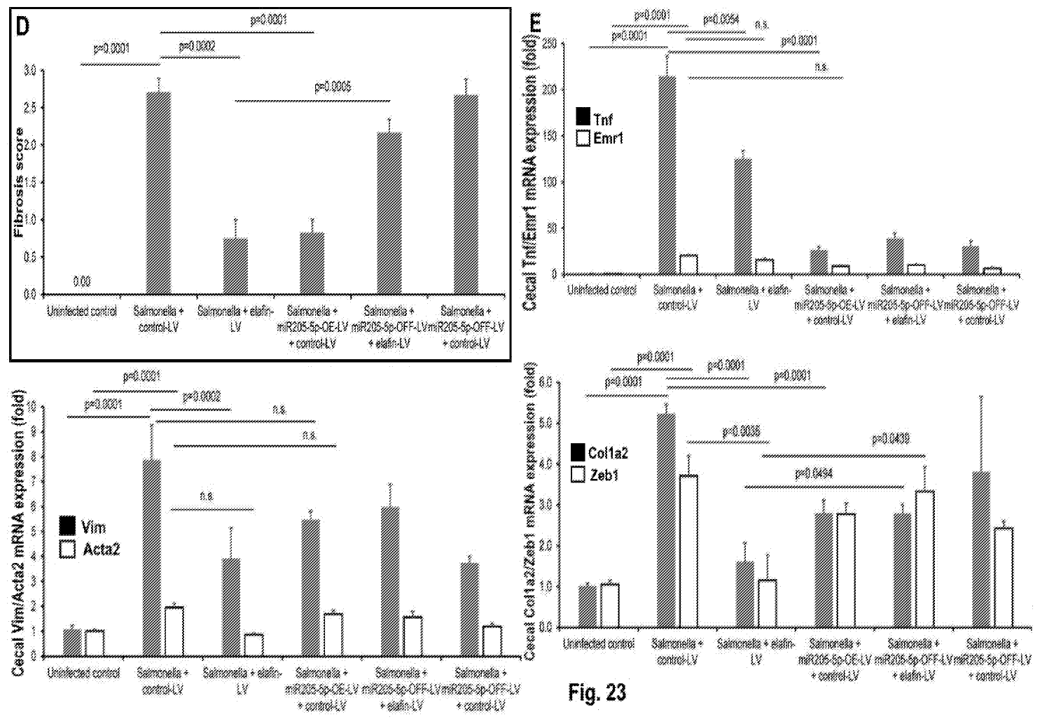

The TNBS-treated mice showed increased colonic Col1a2, Col3a1, Acta2, Vim, Tnf, and Emr1 mRNA expression (FIG. 18E). Anti-TNFa neutralizing antibody ameliorated colonic injury but failed to reduce colonic fibrosis (FIG. 18B-D). The miR205-5p inhibitor specifically reversed elafin-mediated inhibition of colonic fibrosis and collagen (Col1a2 and Col3a1) and Zeb1 mRNA expression (FIG. 18B, 18D-E). Similarly, the miR205-5p-OFF lentivirus reversed elafin-mediated inhibition of cecal fibrosis and Zeb1mRNA expression in Salmonella-infected mice (FIG. 23). Lentiviral miR205-5p overexpression ameliorated colonic and cecal injury and fibrosis in TNBS-treated (FIG. 18B-D) and Salmonella-infected mice (FIG. 23), respectively. Thus, miR205-5p mediates the anti-fibrogenic effect of elafin.

Elafin mediated the anti-fibrogenic effect via ZEB1 inhibition in TNBS-treated and Salmonella-infected mice.

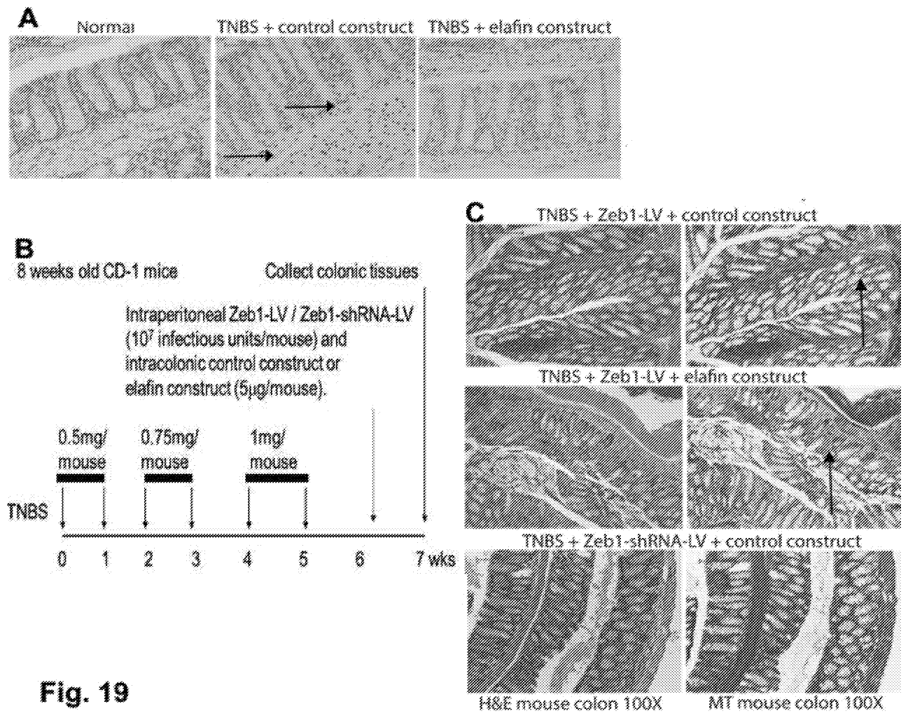

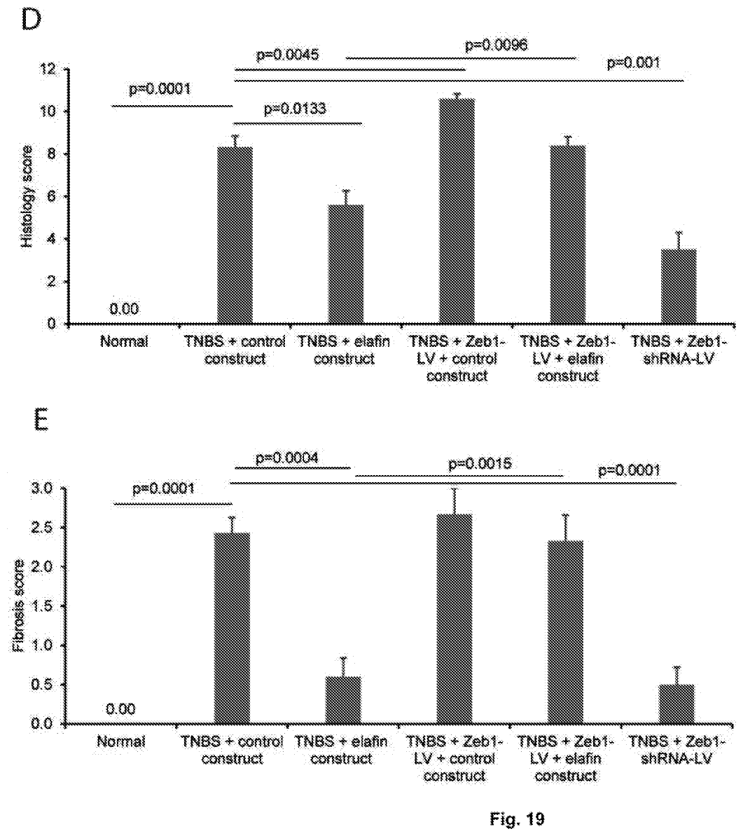

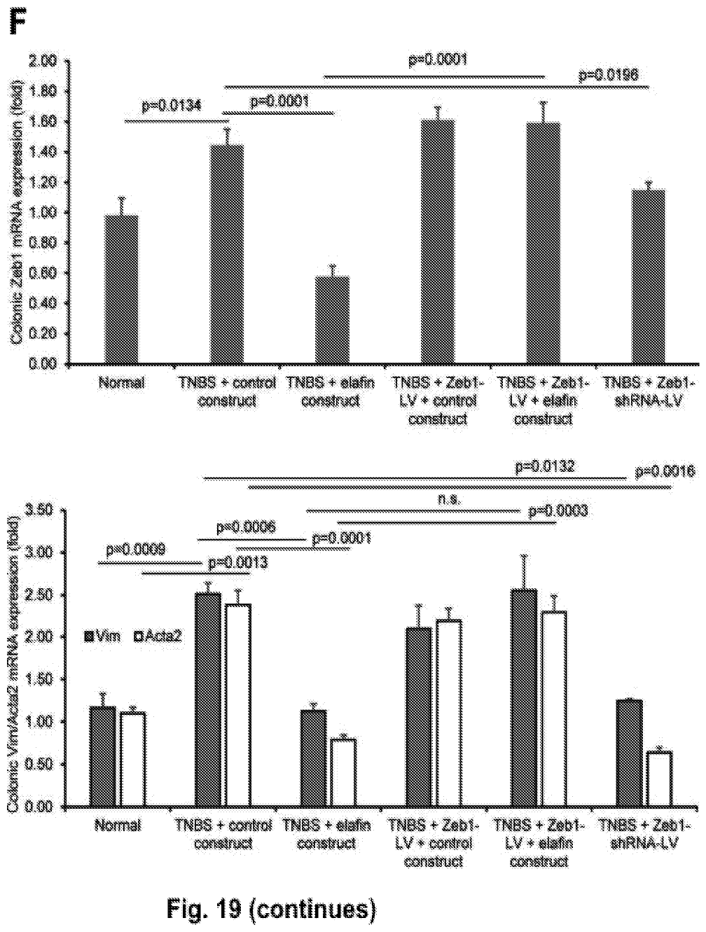

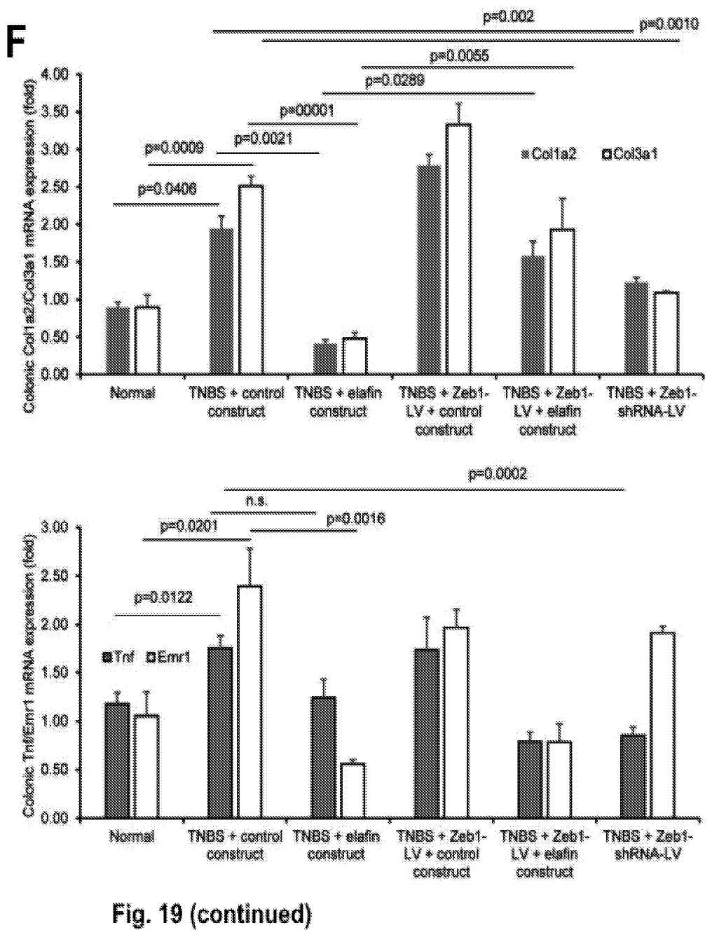

Elafin overexpression diminished colonic ZEB1 immunoreactivity in the TNBS-treated mice (FIG. 19A). We injected Zeb1-overexpressing lentivirus into the TNBS-treated mice to determine the involvement of ZEB1 in the anti-fibrogenic effect of elafin (FIG. 19B). Lentiviral Zeb1overexpression abolished the elafin-mediated inhibition of colonic injury, fibrorsis, and fibrogenic genes (Zeb1, Col1a2, Col3a1, and Acta2) (FIG. 19C-F). Zeb1shRNA lentivirus significantly reduced colonic histology and fibrosis scores and fibrogenic gene mRNA expression (FIG. 19C-F).

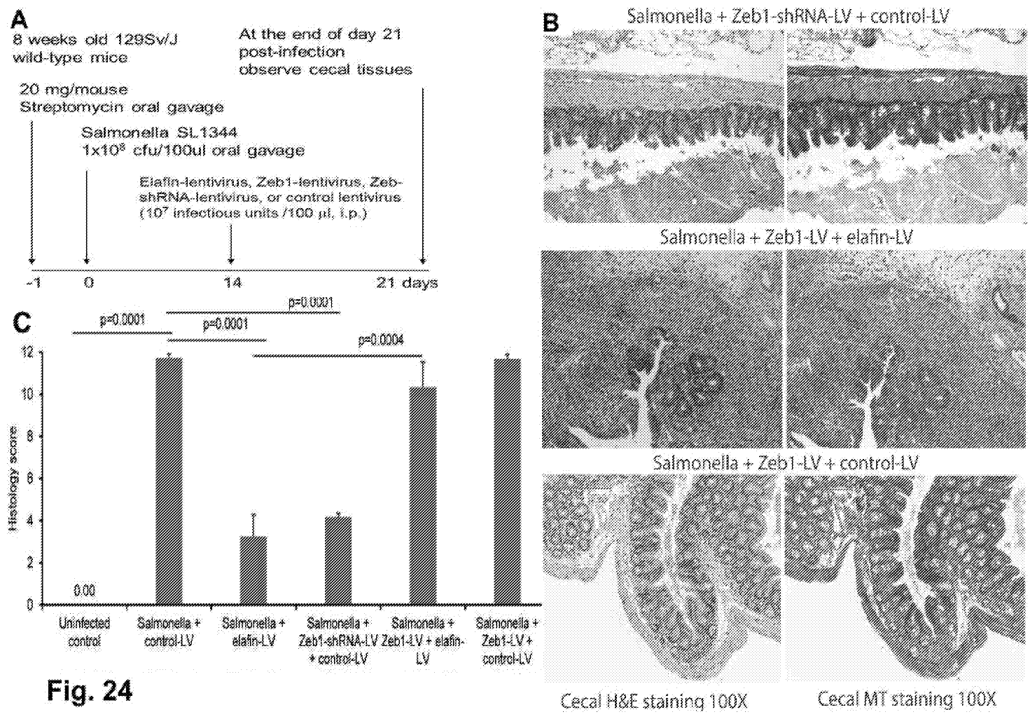

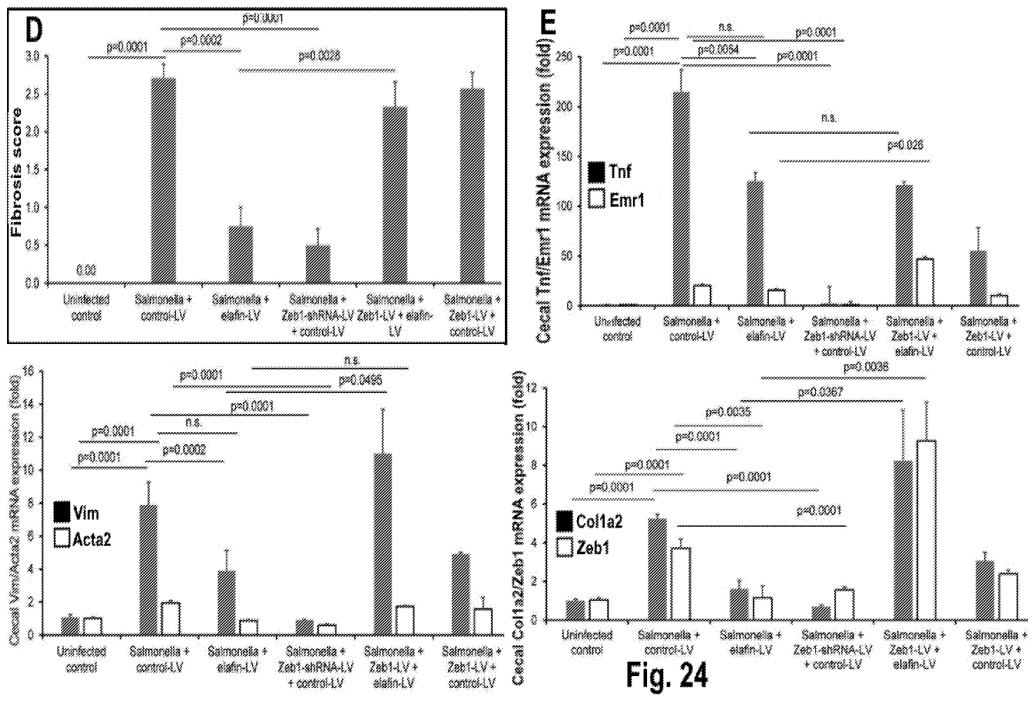

Similarly, Zeb1-shRNA lentivirus ameliorated cecal injury and fibrosis, while Zeb1-overexpressing lentivirus reversed elafin-mediated inhibition of cecal collagen deposition and Zeb1, Vim, and Col1a2 mRNA expression in Salmonella-infected mice (FIG. 24). The anti-fibrogenic effect of elafin is mediated by Zeb1 inhibition.

Elafin mediated anti-fibrogenic effect via cathepsin S-dependent PAR2 inhibition in fibroblasts.

Elafin inhibits elastase-mediated protease-activated receptor 2 (PAR2) activity [43], but the involvement of PAR2 in CD-associated intestinal fibrosis is unknown. PAR2 also activates ERK activity [44]. PAR2 inhibitor GB88 diminished ERK1/2 phosphorylation, while PAR2 agonist reversed elafin-mediated inhibition of ERK1/2 phosphorylation in human colonic CCD-18Co fibroblasts (FIG. 27B-C).

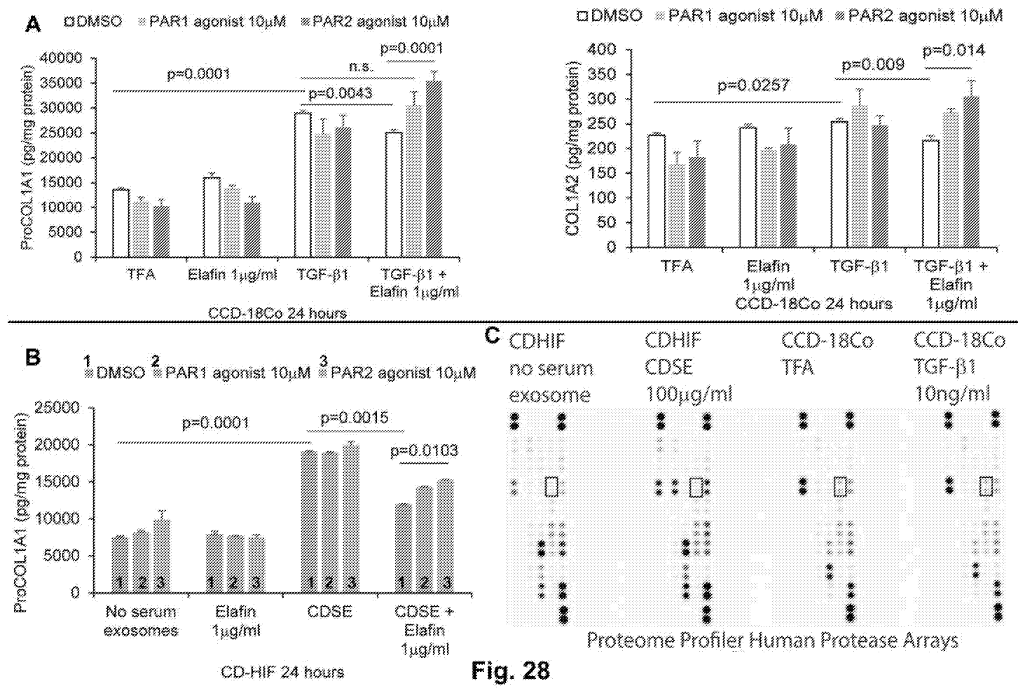

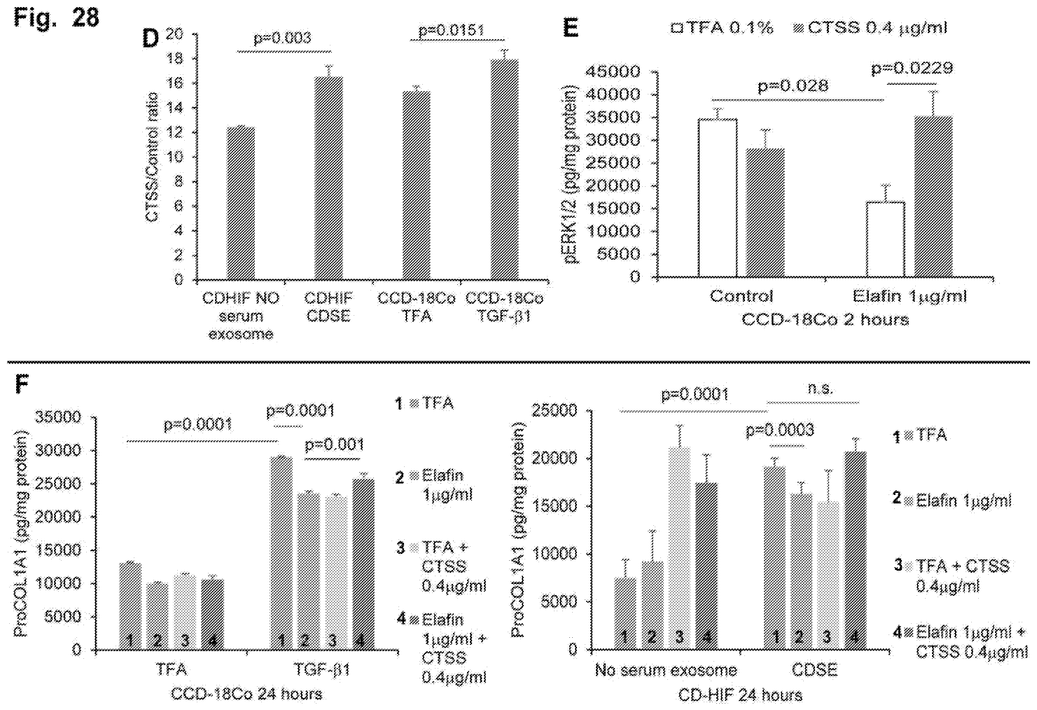

PAR2 can inhibit miR205 expression [45]. PAR2 agonist, but not PAR1 agonist, reversed elafin-mediated miR205-5p induction and ZEB1 inhibition in the TGF-β1-treated CCD-18Co fibroblasts (FIG. 27D-E). GB88 inhibited collagen expression (FIG. 27F), while PAR2 agonist also reversed elafin-mediated inhibition of collagen expression in TGF-β1-treated CCD-18Co fibroblasts and CDSE-conditioned primary human colonic fibroblasts (FIG. 28A-B).

Protease arrays indicated that both CDSE and TGF-β1 consistently induced cathepsin S secretion in the conditioned media of primary stricturing CD and CCD-18Co colonic fibroblasts (FIG. 28C-D). Cathepsin S, a PAR2-activating protease [46], reversed elafin-mediated inhibition of ERK1/2 phosphorylation and ProCOL1A2 expression in colonic fibroblasts (FIG. 28E-F). Thus, elafin inhibits fibrogenesis via cathepsin S-dependent PAR2 suppression.

Elafin inhibited intestinal fibrosis via PAR2 inhibition in TNBS-treated and Salmonella-infected mice.

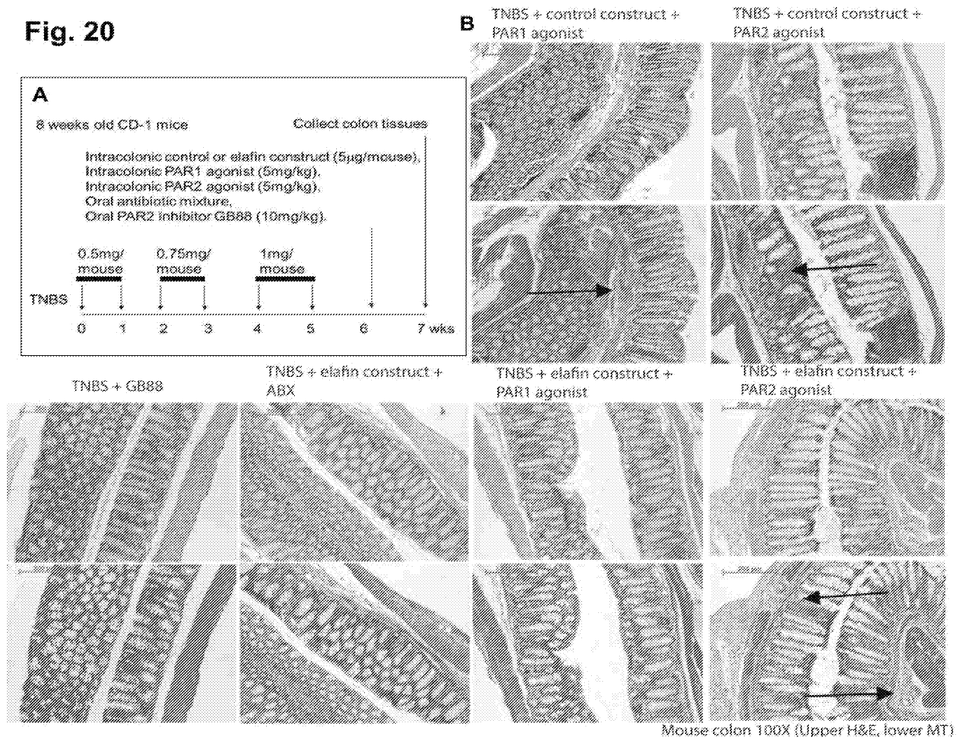

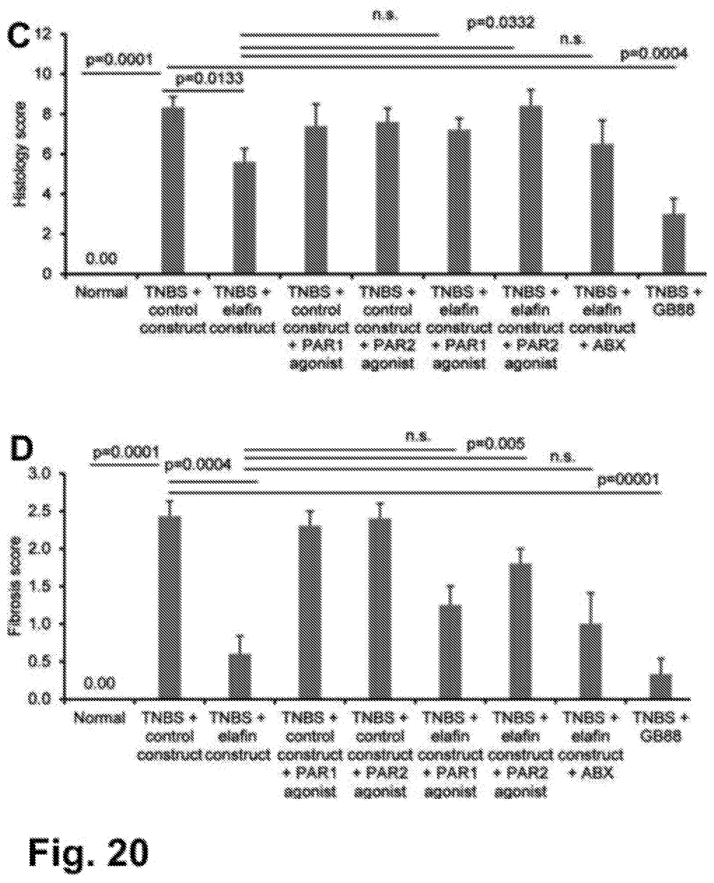

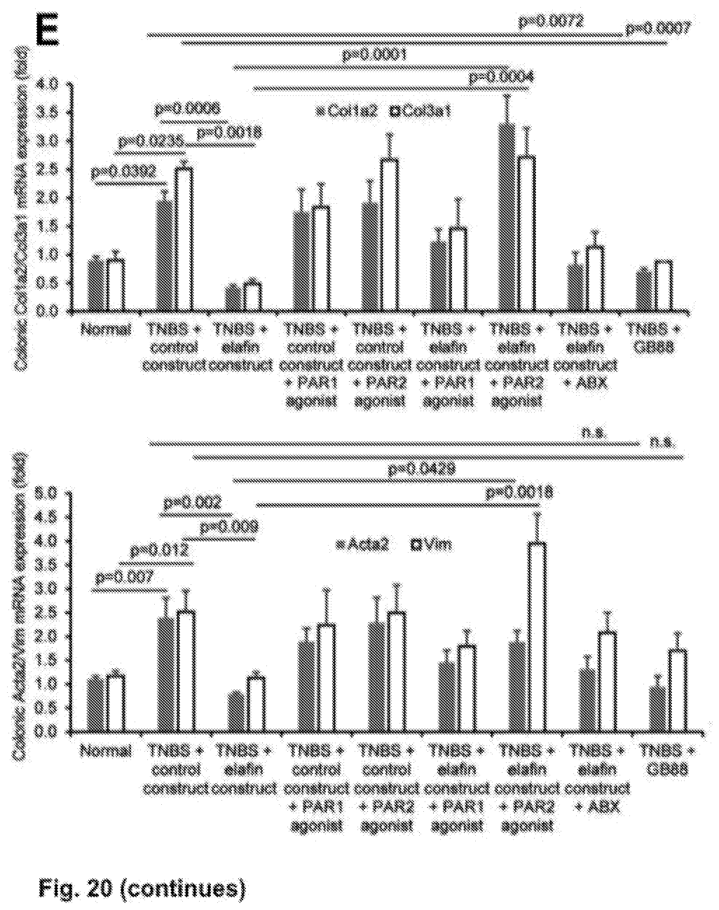

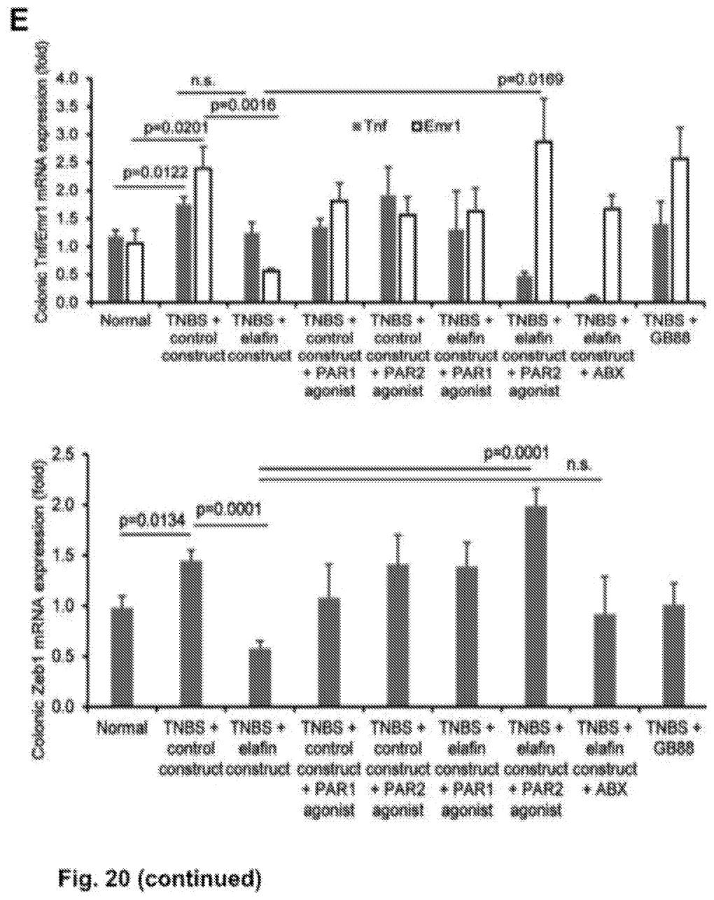

To evaluate the involvement of PAR2 in the anti-fibrogenic effects of elafin in vivo, we injected PAR1 and PAR2 agonists into the TNBS-treated mice intracolonically (FIG. 20A). Both agonists did not affect TNBS-mediated colonic injury and fibrosis in mice (FIG. 20B-C). PAR2, but not PAR1 agonist diminished colonic miR205-5p expression (FIG. 29D), and reversed elafin-mediated inhibition of colonic injury and fibrosis (FIG. 20C-E) with increased colonic Col1a2, Col3a1, Zeb1, Acta2, Vim, and Emr1 mRNA expression (FIG. 20E).

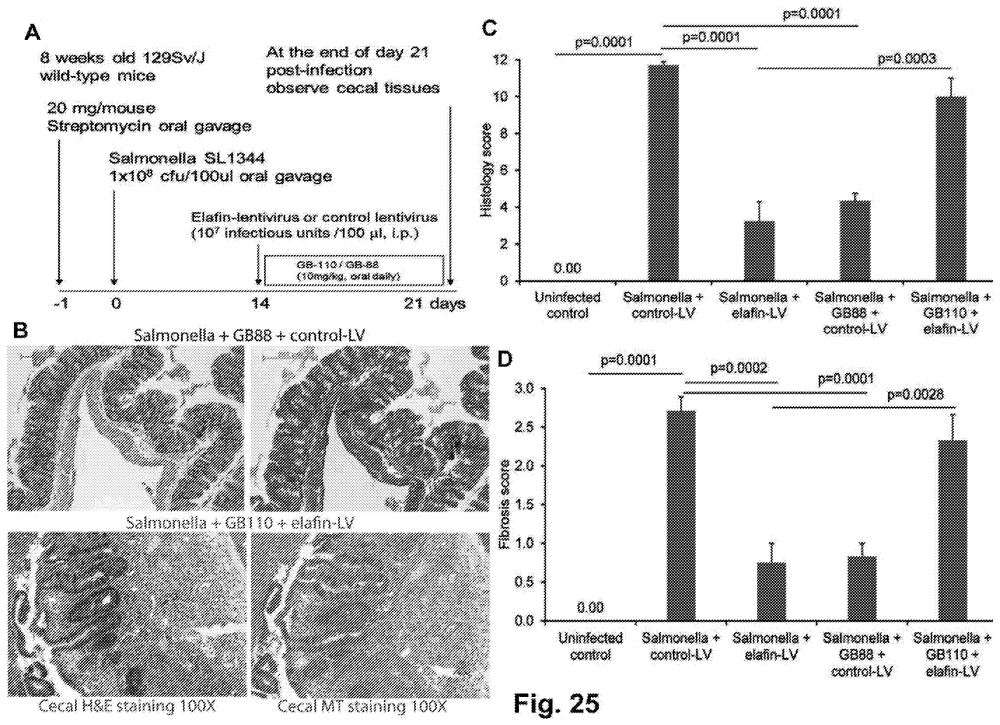

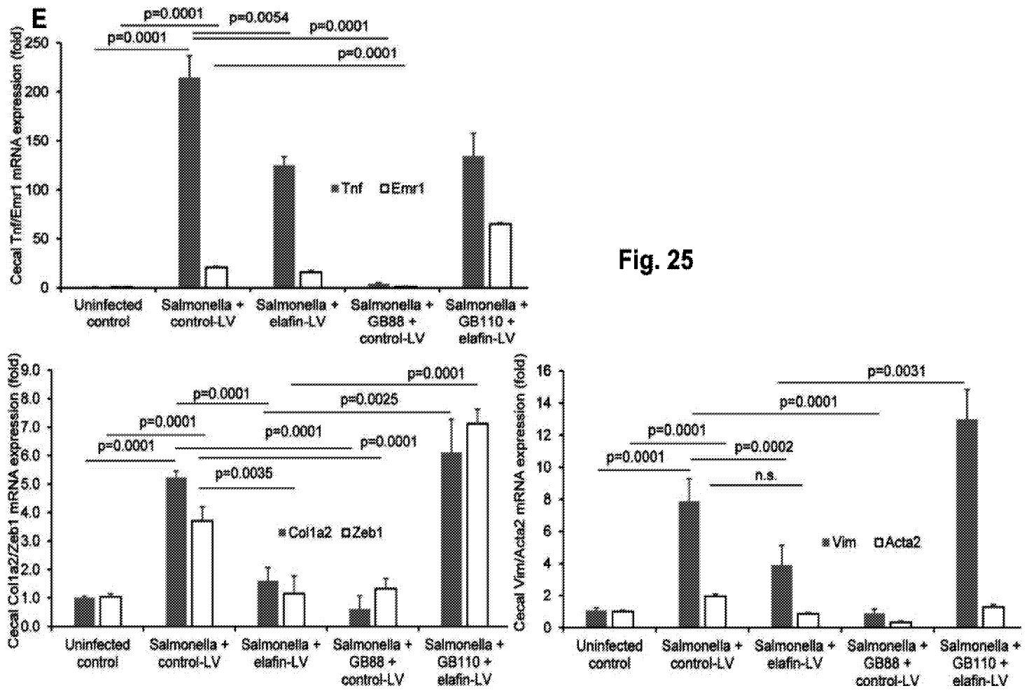

GB88 ameliorated colonic/cecal injury and fibrosis in TNBS-treated mice and Salmonella-infected mice, respectively (FIG. 20B-E and FIG. 25). PAR2 agonist (GB110) 5 reversed the elafin-mediated inhibition of cecal collagen deposition and Col1a2, Zeb1, Vim mRNA expression in Salmonella-infected mice (FIG. 25). Thus, elafin reversed intestinal fibrosis via PAR2 inhibition.

Oral elafin-Eudragit-HPMC formulation inhibits intestinal fibrosis in mice.

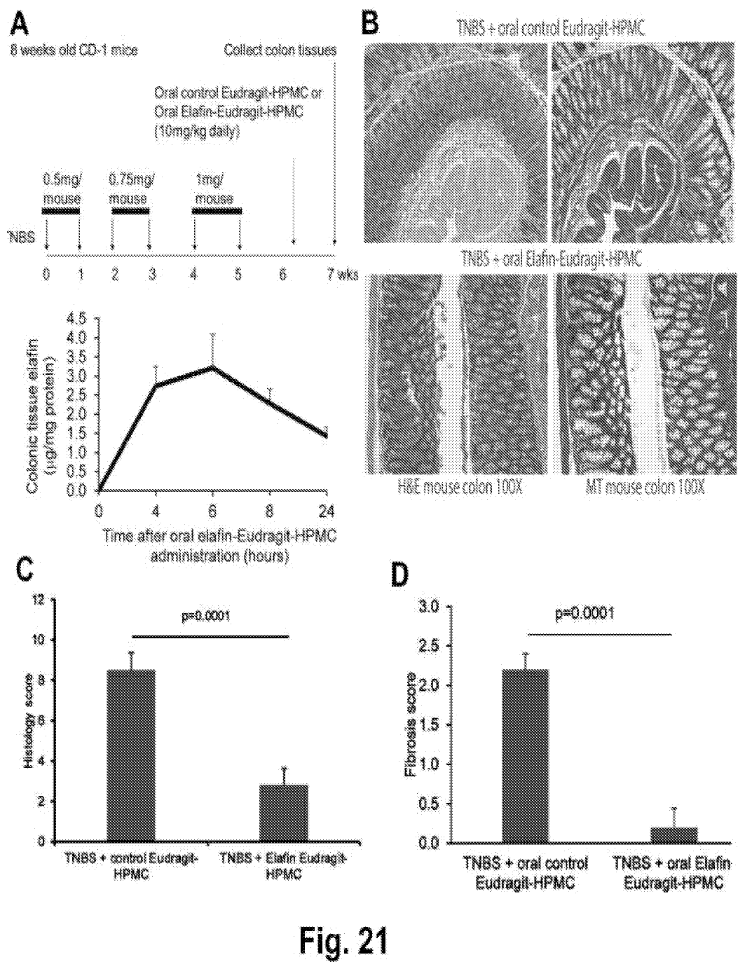

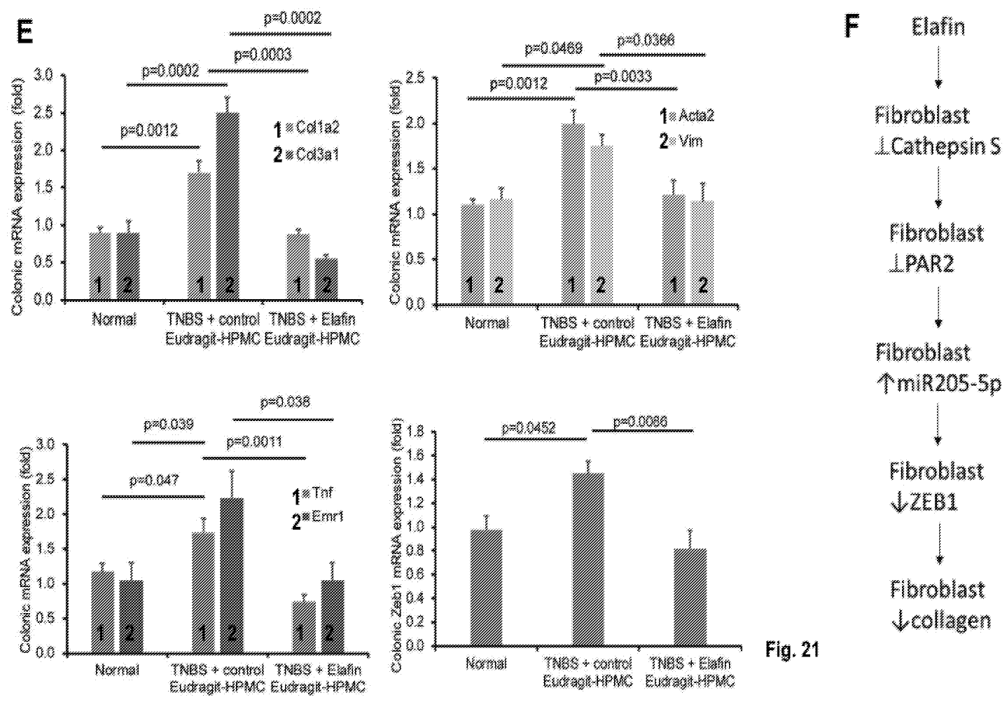

We generated a clinically relevant elafin-Eudragit-HPMC formulation for oral administration (FIG. 21A) [24, 27]. Colonic elafin reached the peak level at 6 hours after oral gavage (FIG. 21A). Oral elafin-Eudragit-HPMC administration reversed colonic injury and fibrosis (FIG. 21B-D), increased colonic miR205-5p expression (FIG. 29E), and reduced colonic mRNA expression of fibrogenic and inflammatory genes in the TNBS-treated mice (FIG. 21E).

The anti-fibrogenic effect of elafin is independent of the gut microbiome

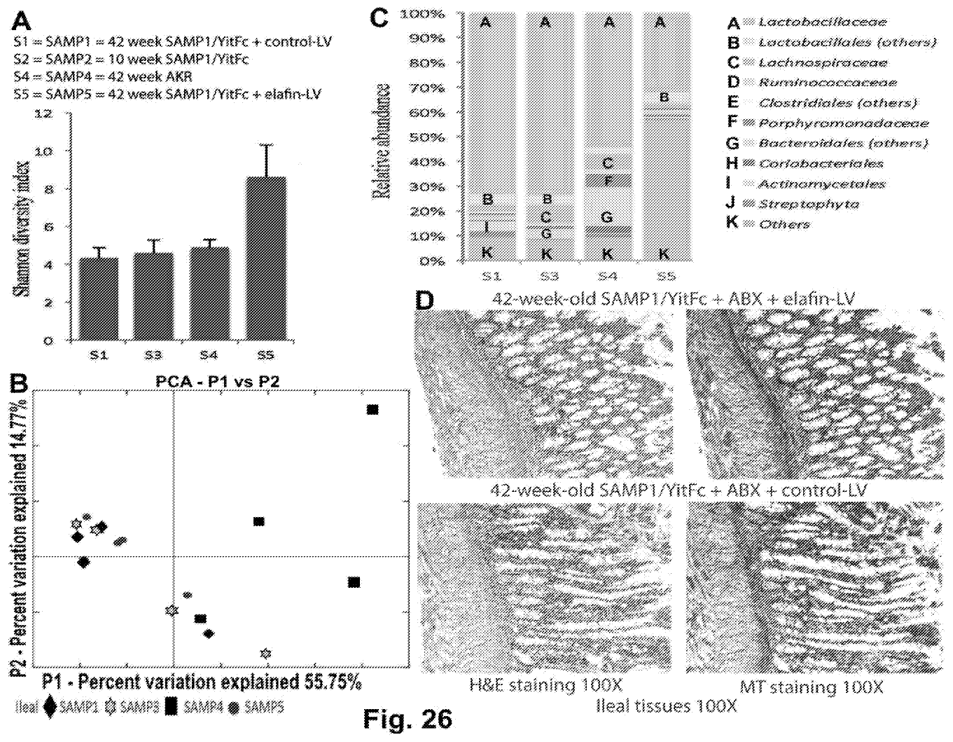

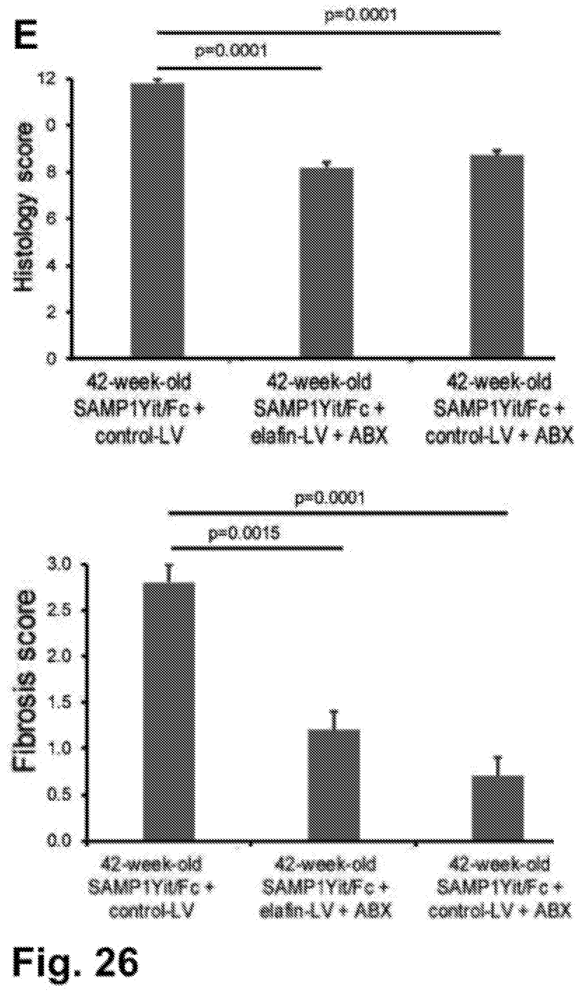

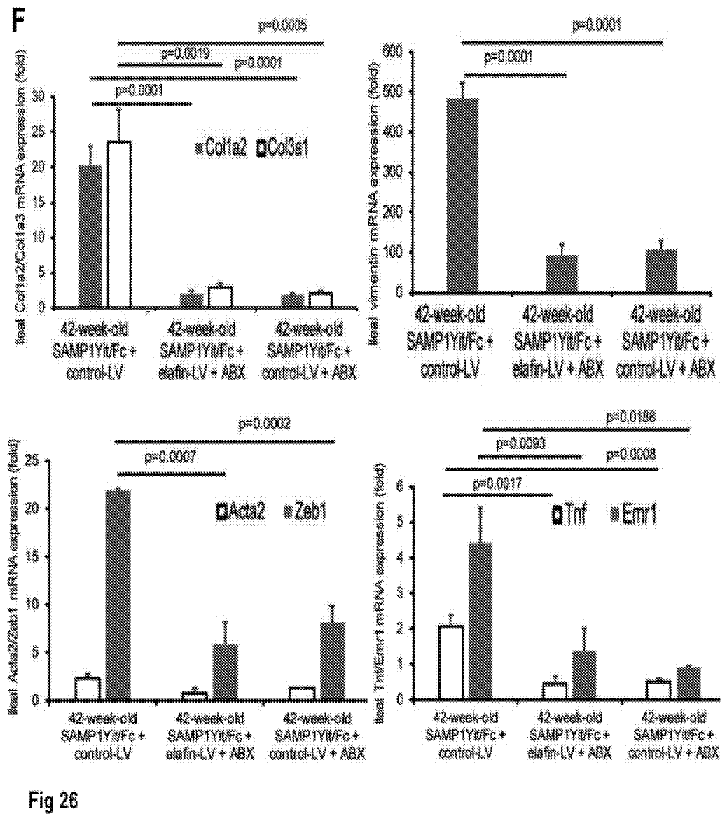

We determined the ileal microbiome in SAMP1/YitFc mice using 16S ribosomal RNA (rRNA) sequencing. Compared to normal AKR mice (S4), both 10-week-old SAMP1/YitFc mice (S2) and 42-week-old SAMP1/YitFc mice (S1) had no significant change of alpha diversity (FIG. 26A). Lentiviral elafin overexpression increased ileal species diversity in the 42-week-old SAMP1/YitFc mice (S5 versus S1), but the difference was statistically insignificant (FIG. 26A). The 42-week-old fibrotic (SAMP1) and 10-week-old (SAMP2) non-fibrotic SAMP1/YitFc mice had a similar ileal microbiome beta diversity, buth both were different from those in AKR normal mice SAMP4(FIG. 26B). Elafin overexpression did not affect ileal microbiome beta diversity in 42-week-old SAMP1/YitFc mice (SAMP1 versus SAMPS; FIG. 26B). Ileal Lactobacillaceae is dominant in SAMP1/YitFc mice [47], but the elafin-mediated reduction of ileal Lactobacillaceae (S1 versus S5) was statistically insignificant (FIG. 26C).

As short-term antibiotics treatment ameliorated ileitis in young SAMP1/YitFc mice [48], long-term antibiotics treatment from 10th to 42nd week of age reduced ileal fibrosis in SAMP1/YitFc mice (FIG. 26D-F). Short-term antibiotics treatment did not affect cecal fibrosis in Salmonella-infected mice (FIG. 22) [35] and colonic fibrosis in TNBS-treated mice [24]. Antibiotics did not affect elafin's anti-fibrogenic effects in three mouse models (FIG. 20E-I and FIGS. 22 and 26).

Discussion

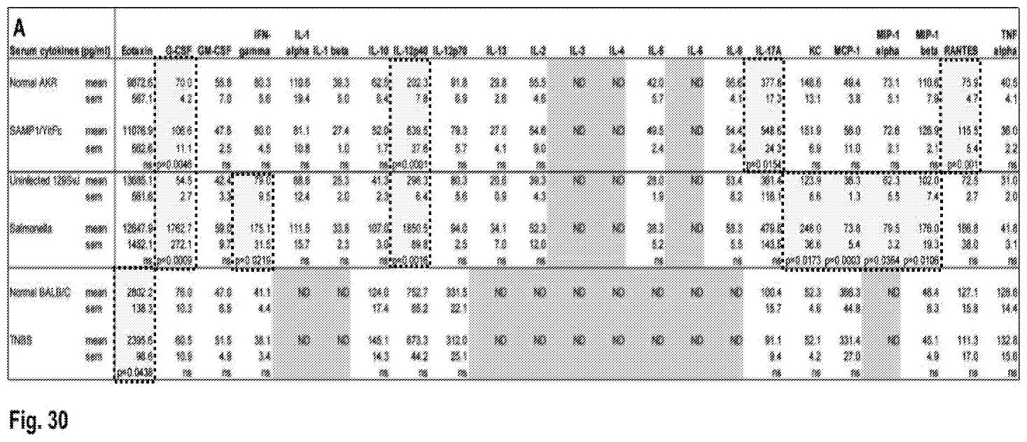

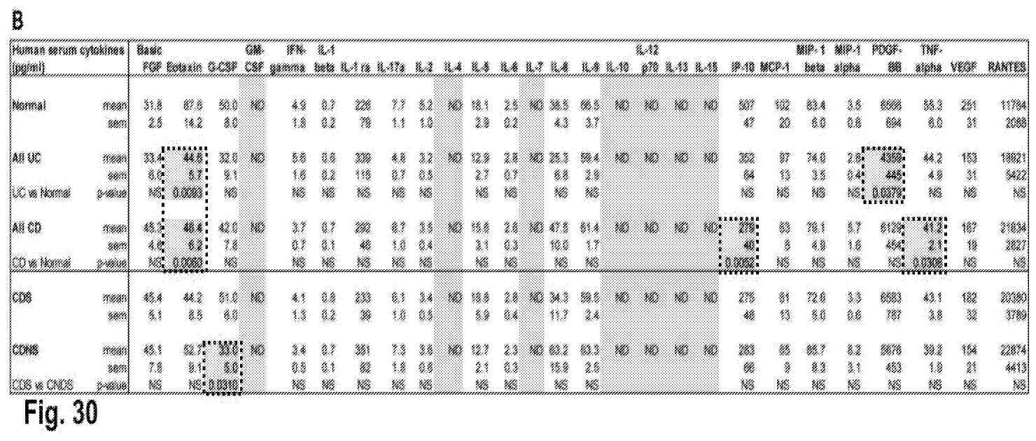

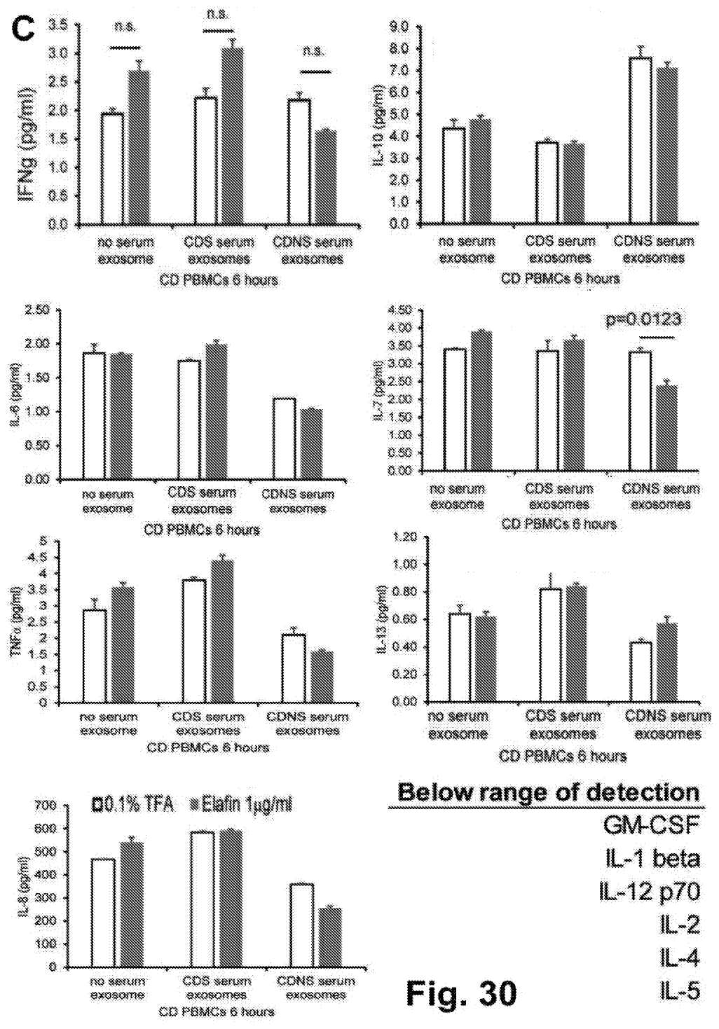

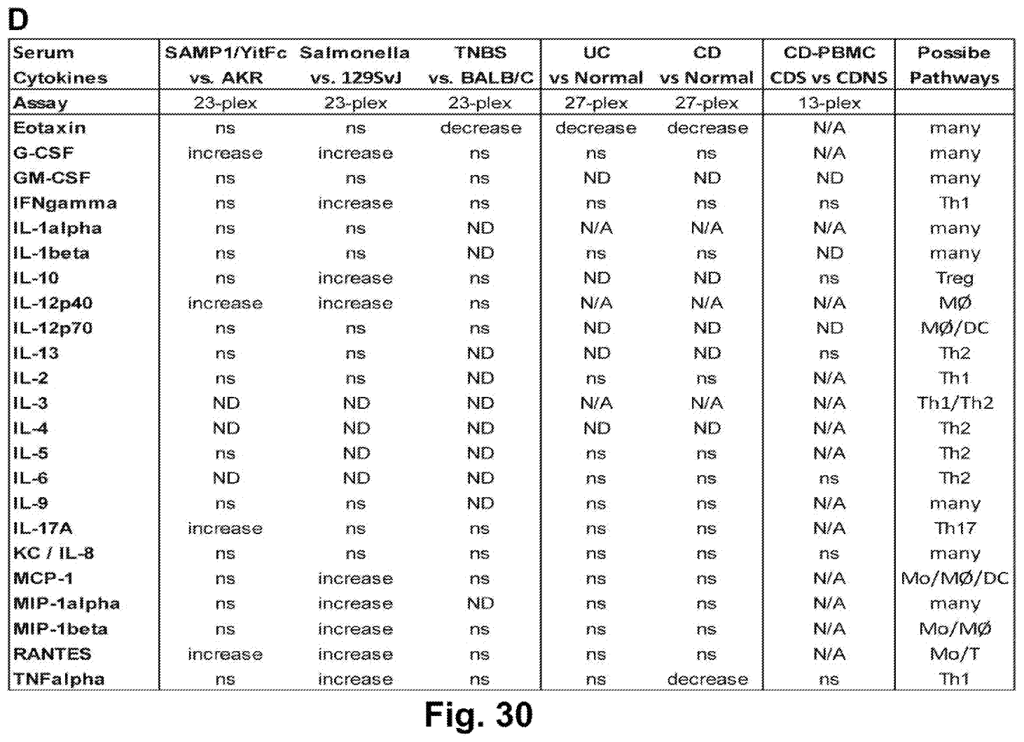

This report is the first to identify elafin, cathepsin S, PAR2, miR205-5p, and ZEB1 as therapeutic targets against intestinal fibrosis. Elafin robustly inhibited collagen expression in two human colonic fibroblasts and reversed preexisting intestinal fibrosis among three mouse models. The circulating cytokine profiles among three mouse models and IBD patients are inconsistent (FIG. 30A-B, D). Elafin did not affect T-cell cytokine secretion in CDSE-preconditioned CD PBMCs (FIG. 30C). The anti-fibrogenic effects of elafin are not associated with circulating cytokines.

PAR2 promotes inflammation and fibrosis [43, 49-51]. Elafin reversed fibrogenesis via specific inhibition of PAR2-activating elastase and protease cathepsin S (FIGS. 27-28). Many proteases can activate PAR2 [52]. Elafin may inactivate PAR2 activity by inhibiting other proteases in IBD patients [53, 54].

Exogenous elafin reverses intestinal fibrosis via PAR2-mediated miR205-dependent ZEB1 inhibition (FIGS. 17C-D, 19-20, and FIGS. 23-25). Elafin, via PAR2 inhibition, increased miR205-5p expression (FIG. 17E and FIG. 27E) because PAR2 inhibits miR205 expression [45]. miR205 subsequently suppresses ZEB1 expression [40]. Intestinal stricture in CD patients is positively correlated with colonic ZEB1 expression (FIG. 17A-B) [12], but not associated with colonic miR205-5p expression (FIG. 29F) because all CD patients have low colonic endogenous elafin expression [9, 12].

The intestinal microbiome has no strong association with strictures in CD patients [2]. Although Ruminococcus is associated with stricturing phenotype in CD patients [2], SAMP1/YitFc mice had low ileal Ruminococcaceae abundance (FIG. 26C). There is no evidence suggesting the association between Lactobacillaceae with intestinal fibrosis.

Consistent with our recent study [55], oral elafin-Eudragit FS30D-HPMC formulation was effective against intestinal fibrosis (FIG. 21) because the Eudragit-FS3OD polymer protected the elafin through the stomach and then released it in intestine [56-58]. Asacol is a Eudragit-coated mesalamine for treating IBD [59]. The oral cathelicidin mimic CSA13-Eudragit-FS3OD formulation ameliorates intestinal fibrosis and C. difficile infection in mice [24, 27].

Intestinal strictures are classified into inflammatory, fibrotic, and mixed phenotypes [60]. Inflammatory strictures can be treated with anti-inflammatory drugs, but fibrotic strictures have no known anti-fibrogenic drugs. Imaging analysis is inaccurate in differentiating stricture phenotypes, while ileocolonoscopy may be unable to gain access to the strictures for evaluation, especially to sites with multiple strictures [60]. It is unfeasible to define phenotype-based therapy in current clinical practice because most CD patients' phenotypes are unknown. Therefore, elafin therapy should cover all phenotypes.

CD disease activity scoring systems suggest that it is necessary to reduce severe disease activity to 33-24% to achieve remission [61]. Overall disease activity assessment provides an objective approach to compare the efficacies of elafin across mouse models. Elafin diminished the overall disease activity to 7% in SAMP1/YitFc model, 29% in Salmonella model, and 7-11% in TNBS models.

In summary, elafin inhibits cathepsin S-dependent PAR2 activity, induces miR205-5p expression, and reduces ZEB1 and collagen expression in intestinal fibroblasts (FIG. 21F).

Elafin overexpression and the orally active elafin-Eudragit-HPMC formulation are highly effective against intestinal fibrosis in mice.

### REFERENCES

1. Lenti M V, Di Sabatino A. Mol Aspects Med 2019; 65:100-109.
2. Kugathasan S, et al. Lancet 2017; 389:1710-1718.
3. Ho S, Pothoulakis C, Koon H W. Curr Pharm Des 2013; 19:40-7.
4. Hing TC, et al. Gut 2013; 62:1295-305.
5. Tai E K, et al. Exp Biol Med (Maywood) 2007; 232:799-808.
6. Yoo J H, et al. Cell Mol Gastroenterol Hepatol 2015; 1:55-74 el.
7. Shaw L, Wiedow O. Biochem Soc Trans 2011; 39:1450-4.
8. Wiedow O, et al. J Biol Chem 1990; 265:14791-5.
9. Zhang W, et al. Inflamm Bowel Dis 2017; 23:2134-2141.
10. Zhang W, et al. Zhonghua Yi Xue Za Zhi 2016; 96:1120-3.
11. Flach CF, et al. Inflamm Bowel Dis 2006; 12:837-42.
12. Wang J, et al. PLoS One 2020; 15:e0231796.
13. Schmid M, et al. J Leukoc Biol 2007; 81:907-15.
14. Motta J P, et al. Gastroenterology 2011; 140:1272-82.
15. Bermudez-Humaran LG, et al. Microb Cell Fact 2015; 14:26.
16. Galipeau HJ, et al. Am J Gastroenterol 2014; 109:748-56.
17. Doron S, Snydman DR. Clin Infect Dis 2015; 60 Suppl 2:S129-34.
18. Sotoudegan F, et al. Food Chem Toxicol 2019.
19. Alam S R, et al. Heart 2015; 101:1639-45.
20. Pizarro T T, et al. Inflamm Bowel Dis 2011; 17:2566-84.
21. Kosiewicz M M, et al. J Clin Invest 2001; 107:695-702.
22. Grassi G A, et al. Gastroenterology 2008; 134:768-80.
23. Lawrance I C, et al. Gastroenterology 2003; 125:1750-61.
24. Xu C, et al. Sci Rep 2017; 7:16351.
25. Xu T, et al. BMC Public Health 2017; 17:337.
26. Koon HW, et al. Am J Pathol 2011; 179:2315-26.
27. Wang J, et al. Gastroenterology 2018; 154:1737-1750.
28. Strober W, et al. J Clin Invest 2001; 107:667-70.
29. Human Microbiome Project C. A framework for human microbiome research. Nature 2012; 486:215-21.
30. DeSantis T Z, et al. Appl Environ Microbiol 2006; 72:5069-72.
31. Langille M G, et al. Nat Biotechnol 2013; 31:814-21.
32. Langmead B, Salzberg SL. Nat Methods 2012; 9:357-9.
33. Caporaso J G, et al. Nat Methods 2010; 7:335-6.
34. Kruschewski M, et al. Dig Dis Sci 2001; 46:2336-43.
35. Johnson L A, et al. Inflamm Bowel Dis 2012; 18:460-71.
36. Koon H W, et al. Gastroenterology 2011; 141:1852-63 e1-3.
37. Wu F, Chakravarti S. J Immunol 2007; 179:6988-7000.
38. Masterson J C, et al. Am J Pathol 2011; 179:2302-14.
39. Johnson L A, et al. J Crohns Colitis 2017; 11:724-736.
40. Gregory P A, et al. Nat Cell Biol 2008; 10:593-601.
41. Peng D H, et al. Oncogene 2017; 36:1925-1938.
42. Li L, Li S. Oncol Lett 2018; 16:1715-1721.
43. Zhao P, et al. J Biol Chem 2015; 290:13875-87.
44. Jimenez-Vargas N N, et al. Proc Natl Acad Sci U S A 2018; 115:E7438-E7447.
45. Yang X, et al. Cancer Invest 2017; 35:601-609.
46. Zhao P, et al. J Biol Chem 2014; 289:27215-34.
47. Rodriguez-Palacios A, et al. Inflamm Bowel Dis 2018; 24:1005-1020.
48. Bamias G. et al. J Immunol 2002; 169:5308-14.
49. Shearer A M, et al. J Biol Chem 2016; 291:23188-23198.
50. Knight V, et al. Hepatology 2012; 55:879-87.
51. Lohman RJ, et al. J Pharmacol Exp Ther 2012; 340:256-65.
52. Heuberger DM, Schuepbach RA. Thromb J 2019; 17:4.
53. Denadai-Souza A, et al. Sci Rep 2018; 8:7834.
54. Townsend P, et al. Inflamm Bowel Dis 2015; 21:1935-41.
55. Wang J, et al. Sci Rep 2020; 10:12785.
56. Huyghebaert N, et al. Eur J Pharm Biopharm 2005; 61:134-41.
57. Naeem M, et al. Int J Nanomedicine 2018; 13:1225-1240.
58. Poelvoorde N, et al. Eur J Pharm Biopharm 2008; 69:969-76.
59. Faber S M, Korelitz B I. J Clin Gastroenterol 1993; 17:213-8.
60. Bettenworth D, et al. World J Gastroenterol 2016; 22:1008-16.
61. Peyrin-Biroulet L, et al. Clin Gastroenterol Hepatol 2016; 14:348-354 e17.

### Example 3

### Elafin Inhibits Obesity, Hyperglycemia, and Liver Steatosis in High-Fat Diet-Treated Male Mice

Elafin is an antimicrobial and anti-inflammatory protein. We hypothesize that elafin expression correlates with diabetes. Among non-diabetic and prediabetic groups, men have significantly higher serum elafin levels than women. Men with type 2 diabetes mellitus (T2DM) have significantly lower serum elafin levels than men without T2DM. Serum elafin levels are inversely correlated with fasting blood glucose and hemoglobin A1c levels in men with T2DM, but not women with T2DM. Lentiviral elafin overexpression inhibited obesity, hyperglycemia, and liver steatosis in high-fat diet (HFD)- treated male mice. Elafin-overexpressing HFD-treated male mice had increased serum leptin levels, and serum exosomal miR181b-5p and miR219-5p expression. Transplantation of splenocytes and serum exosomes from elafin-overexpressing HFD-treated donor mice reduced food consumption and fat mass, and increased adipose tissue leptin mRNA expression in HFD-treated recipient mice. Elafin improved leptin sensitivity via reduced interferon-gamma expression and induced adipose leptin expression via increased miR181b-5p and miR219-5p expression. Subcutaneous and oral administration of modified elafin inhibited obesity, hyperglycemia, and liver steatosis in the HFD-treated mice. Circulating elafin levels are associated with hyperglycemia in men with T2DM. Elafin, via immune-derived miRNAs and cytokine, activates leptin sensitivity and expression that subsequently inhibit food consumption, obesity, hyperglycemia, and liver steatosis in HFD-treated male mice.

The Centers for Disease Control and Prevention (CDC) reported that 8.6% of U.S. adults are diagnosed with type 2 diabetes mellitus (T2DM)[1]. T2DM is characterized by hyperglycemia with a combination of insulin resistance and relative insulin deficiency. Globally, more men are diagnosed with T2DM than women [2,3]. The development of T2DM involves many factors and is a topic of intense research [4].

Recent reports suggest the relevance of antimicrobial peptides (cathelicidin and lactoferrin) in the regulation of diabetes [5-7]. Interestingly, one of the antimicrobial peptides, elafin, is abundantly expressed in the urinary extracellular vesicles in patients with type I diabetes mellitus (T1DM) [8]. Its level is progressively decreased when these patients develop diabetic nephropathy, suggesting its involvement in diabetes. Elafin is a small (6 kDa) human elastase-specific protease inhibitor and antimicrobial peptide primarily expressed in immune cells, intestinal tract, vagina, lungs, and skin [9,10]. Circulating elafin levels are positively correlated with inflammatory bowel disease and graft-versus-host disease [10,11]. Elafin expression is also increased in human atherosclerotic coronary arteries and mesenteric fat in stricturing Crohn's disease patients [10,12]. However, the circulating levels of elafin in patients with T2DM are unknown.
Elafin possesses anti-inflammatory effects as elafin inhibits lipopolysaccharide (LPS)-mediated inflammatory responses including activator protein 1 (AP-1) and Nuclear Factor kappa-light-chain-enhancer of activated B cells (NF-KB) in monocytes [13]. Elafin also reduces interleukin-8 (IL-8) production in endothelial cells exposed tooxidized low-density lipoprotein (oxLDL) [14]. In utilizing elafin for therapeutic applications, subcutaneous injections of elafin can reverse pulmonary hypertension in rats [15]. Additionally, oral administration of elafin-expressing Lactococcus ameliorates dextran sulfate (DSS)- and trinitrobenzene sulfonic acid (TNBS)-mediated colitis in mice and gluten-related disorders in humans [16,17]. However, the therapeutic potential of elafin in diabetes is unknown.

As antimicrobial peptides are associated with diabetes, we hypothesize that a link between elafin expression and diabetes may exist. Our study included a cohort of patients for determining the serum elafin levels in non-diabetic, prediabetic, and diabetic (T2DM) patients. This study discovered the unique clinical significance of abnormal elafin expression in patients with T2DM. We utilized well established high-fat diet (HFD)-treated mice as diet-induced obesity (D10) model for T2DM [18]. Through the application of B- and T-cell deficient Rag−/− mice, splenocyte transplantation, and serum exosome transplantation, this study is also the first to elucidate the mechanistic connection between cytokine, miRNA, and hormone in the elafin-mediated regulation of obesity, hyperglycemia, and liver steatosis in HFD-treated mice. We developed two clinically useful elafin delivery approaches and evaluated their anti-diabetic efficacies in HFD-treated mice.

Methods

Human serum samples. Patients were assigned to non-diabetic, prediabetes, and diabetic groups using the American Diabetes Association criteria [63]. UCLA pathology laboratory collected blood from patients and prepared the sera by centrifugation, following its standard operating procedures (SOPs). Inclusion criteria: Subjects 20-70 years of age. Exclusion criteria: Pregnant women, prisoners, or minors under 18 years old were excluded. Patients with type 1 diabetic mellitus (T1DM) were excluded. This study was a prospective study. The baseline characteristics are shown in Table 51. Tables S1-S3 and FIGS. S1-S8 referenced throughout this Example are all available via the electronic publication of this study at Wang et al. 2020, Scientific Reports 10, Article number: 12785 (2020) doi.org/10.1038/s41598-020-69634-3.

The mean values of blood tests including creatinine (Cr), blood urea nitrogen (BUN), alanine aminotrans- ferase (ALT), aspartate aminotransferase (AST), alkaline phosphatase (ALP), and albumin (ALB) are within normal ranges (Table S1). A woman without diabetes, a man with prediabetes, three men with T2DM, and a woman with T2DM were diagnosed with non-alcoholic fatty liver disease (NAFLD) (Table S1). No NASH, hepatic fibrosis, or other hepatic abnormalities are noted in the clinical data among all other patients.

All patients with T2DM are not treatment naïve. 82%, 18%, and 5% of the patients with T2DM used metformin, sulfonylureas, and insulin, respectively. None of the non-diabetic or prediabetic patients used metformin, sulfonylureas, or insulin. There is no strong correlation between the use of anti-diabetic drugs and serum elafin levels and the gender of the patients with T2DM (Table S2).

ELISA measurement of human serum samples. Elafin (DY1747, R&D Systems), adiponectin (DY1065, R&D Systems), insulin (#90095, Crystal Chem), and leptin (#80968, Crystal Chem) levels were determined using ELISA. Human serum cytokine levels were determined with a 27-plex Multiplex ELISA (#m500kcaf0y, Bio-Rad) using a Bio-plex 3D suspension array system (Bio-Rad) at UCLA Center for Systems Biomedicine [64].

Human mesenteric fat samples. Human mesenteric fat tissues from non-diabetic patients were collected from the Cedars-Sinai Medical Center (CSMC) between 2010-2014 prospectively. Inclusion criteria: The CSMC's gastroenterologists referred the patients to surgical procedures, as medically indicated. Patients with colorectal cancer, inflammatory bowel diseases, intestinal perforation, diverticulitis, colonic inertia, or obesity were included.

Exclusion criteria: Pregnant women, prisoners, or minors under age 18 were excluded. Patients with concurrent acute infection (CMV, C. difficile, and tuberculosis) were excluded. Patients with prediabetes or diabetes (T1DM or T2DM) were not included. The baseline characteristics are shown in Table S3.

Animal experiments. Eight-week-old male C57BL/6J mice (stock #000664), Rag−/− mice (stock #002216), and ob/ob mice (stock #000632) were purchased from Jackson Laboratories and maintained at the UCLA animal facility under standard environmental conditions with a 12-h light period and a 12-h dark period per day at 25° C. room temperature. They were housed in disposable plastic cages with HEPA filtered air circulation, auto-claved bedding, animal chow, and sterile water ad libitum5. Mice were fed with either regular diet (RD) (6% fat, #7013, Harlan Laboratories), high-fat diet (HFD) (45% Kcal from fat; #D12451, Research Diets, Inc.), or high- cholesterol diet (HCD) (Clinton/Cybulsky low-fat diet with 2% cholesterol, #D01101902C, Research Diets, Inc.) ad libitum. The leptin-deficient ob/ob mice were fed with regular chow ad libitum. All mice were randomized and allocated to each cage (4 mice per cage) by animal facility personnel before experiments began.

For systemic elafin overexpression, some groups were injected with control lentivirus (PS100064V, Origene) or elafin-overexpressing lentivirus (RC203136L1V, Origene) intravenously via tail veins (107 infectious units per mouse). Some groups were injected with IL-1β protein 2.5 μg per mouse (#211-11B Peprotech) or IFNγ protein 5 μg per mouse (#315-05 Peprotech) intraperitoneally to increase circulating IL-1β or IFNγ levels.

Leptin sensitivity tests were performed by injecting 1 μg/kg leptin intraperitoneally (#450-31, Peprotech) daily for 3 days. At the endpoint of the experiments, mice were fasted for 6 h and tested for fasting blood glucose and cholesterol levels with a drop of tail vein blood using Freestyle Lite blood glucose meter and test strips (Abbott Diabetes Care) and Accutrend Plus meter and test strips (Roche), respectively. The oral glucose tolerance test (OGTT) was performed by feeding D-glucose (1 g/kg) to fasted mice via oral gavage, and the blood glucose level was measured 2 h after oral glucose feeding. Blood glucose and cholesterol levels are presented as mg/dL. Measurements of fasting blood glucose levels, blood total cholesterol levels, food consumption, and fat/lean mass (EchoMRl) were performed as described in our previous report [5].

Free fatty acid levels in blood were determined by a free fatty acid quantification kit (#ab65341, Abcam). Mouse serum insulin (#90080, Crystal Chem), adiponectin (MRP300, R&D Systems), and leptin (#90030, Crystal Chem) levels were determined by ELISAs. Mouse serum cytokine levels were determined with a 23-plex Multiplex ELISA (#M60009RDPD, Bio-Rad) using a Bio-plex 3D suspension array system (Bio-Rad) at UCLA Center for Systems Biomedicine [64].

Splenocyte transplantation in mice. The 8-week-old HFD-treated male C57BL/6J mice infected with either control lentivirus or elafin-expressing lentivirus were used as donor mice. Spleens of donor mice were gently minced with a syringe pistol in 2 mL ice-cold phosphate-buffered saline (PBS). The splenocytes were filtered through 70 μm cell strainer with 10 ml ice-cold PBS. The cells were centrifuged (1,500 rpm) and resus- pended in 200 μL ice-cold PBS. The splenocytes were then injected into HFD-treated male Rag−/− recipient mice intraperitoneally [65]. The splenocytes from one donor mouse were injected into one recipient mouse.

Cecal microbiota transplantation in mice. The 8-week-old HFD-treated male C57BL/6J mice with infection of either control lentivirus or elafin-expressing lentivirus were used as donor mice. Cecal content from donor mice was collected during dissection and immediately homogenized in ice-cold PBS (1 g/mL), followed by low-speed centrifugation for one minute. The supernatant of cecal material from one donor mouse was transferred to an 8-week-old HFD-treated male C57BL/6J recipient mouse via oral gavage daily (100 μL/mouse/day) for 14 days [61].

Mouse blood cell and serum exosome preparation and transplantation. Blood samples were collected in tubes containing 50 μL/tube of 0.5% ethylenediaminetetraacetic acid (EDTA) at the time of donor mouse dissection. The blood samples were centrifuged at 10,000 g for 5 min at 4° C. The supernatant (serum) samples were used for ELISA assays and serum exosomes extraction. The pellets (blood cells) were resuspended in 10 mL 1× red blood cell lysis buffer (#420301, BioLegend) for 10 min, followed by dilution in PBS. The cell suspension was centrifuged at 8,000 rpm for 5 min at 4° C., and the supernatant was discarded. The immune cell-containing pellets were resuspended and lysed in Qiazol reagent (#79306, Qiagen) for RNA extraction and RT-PCR experiments.

Serum exosomes of donor mice were prepared using total exosome isolation reagent (#4478360, ThermoFisher), and their quantities were then determined by BCA protein assay. The serum exosomes were diluted in PBS and injected into recipient mice intravenously via tail veins (10 μg per mouse).

For inhibition of miRNAs, control (Y100199006), miR-181b-5p (YC10201288-FZA), and miR-219-5p (YC10201241-FZA) inhibitors (Qiagen) were dissolved in PBS. The miRNA inhibitors (10 mg/kg in 100 μL/ mouse) were injected into the exosome recipient mice subcutaneously under brief isoflurane anesthesia, as recommended by Qiagen.

Histological assessment of hepatic steatosis in mouse liver. Hepatic steatosis was assessed with a NAFLD score: parenchymal involvement by steatosis with <5%=0; 5-33%=1; 33-66%=2; and >66%=366. Two locations per mouse were observed by two investigators in a blinded manner.

Oral and subcutaneous administration of modified elafin to mice. Eight-week-old male c57BL/6J (JAX #000664) mice were fed with HFD for 8 weeks to induce obesity and hyperglycemia, followed by administration of Elafin-Eudragit formulation (10 mg/kg) via oral gavage daily or subcutaneous injection of polyethylene glycol conjugated (PEGylated) elafin (3.25 mg/kg) every 48 h to mice for 14 days. Oral Elafin-Eudragit formulation was made by Dr. Xingguo Cheng at the Southwest Research Institute (SWRI), Texas. Elafin was coated with Eudragit FS3OD polymer. This pH-responsive polymer is insoluble in acid but dissolves in a mildly alkaline environment (i.e., pH 7 or above), which is optimal for colonic delivery. Elafin-Eudragit was packaged into microparticles using an SWRI-patented spinning disk atomization technology. This packaging prevented leakage of elafin in acidic, aqueous solution. The oral elafin-Eudragit formulation was dissolved in mildly acidified (pH 5) water containing 0.5% hydroxypropyl methylcellulose (HPMC). PEGylated elafin was made by conjugation to methoxyl-PEG12 (New England Peptide Company). Control groups received either oral Eudragit-HPMC solution or subcutaneous PEG injection.

Cell culture experiments. Mouse 3T3-L1 preadipocytes (#CL-173, ATCC) were cultured in Dulbecco's modified Eagle's medium (DMEM) (#11965-084, ThermoFisher) with 10% fetal bovine serum (FBS) (#10437-028, Life Technologies) and 1% penicillin/streptomycin/glutamine (P/S/G) (#10378-016, Life Technologies) mixture. The preadipocyte differentiation method was described in our previous report5. Differentiated 3T3-L1 adipocytes were serum-starved overnight followed by treatment with 10 ng/ml elafin (E7280, Sigma), 10 μg/ml exosomes, or 75 ng/ml miRNA mimics to study the role of elafin and its dependent molecules in lipid accumulation and gene expression. Control mimic (#479904-001, Qiagen), miR181b-5p mimic (MSY0000673, Qiagen), miR210-3p mimic (MSY0000658, Qiagen), and miR219-5p mimic (MSY0000664, Qiagen) were transiently transfected to 3T3-L1 adipocytes via HiPerfect transfection reagent (#301704, Qiagen) (75 ng/ml via overnight transfection). Adipocyte lipid accumulation was determined using Oil Red O staining, as described previously [5].

Mesenteric fat preadipocytes from colon cancer patients were collected from a previous study and stored in liquid nitrogen [67]. The human preadipocytes were thawed and cultured in DMEM/F12 media containing 10% calf serum and 1% P/S (Invitrogen) until >60% confluence was achieved. The preadipocytes were passaged to 6-well plates (400,000 cells/plate) in DMEM/F12 media containing 10% calf serum and 1% P/S. Two days later, the preadipocytes underwent a differentiation process, as described by our previous report5. The differentiated adipocytes were serum-starved for 6 h, followed by transient transfection with control, miR181b-5p, or miR219-5p mimics (75 ng/ml) via Lipofectamine 3,000 (L3000001, ThermoFisher) overnight. The transfected cells were then incubated in serum-free DMEM media for 6 h. The conditioned media were collected for leptin ELISA.

Primary human peripheral blood mononuclear cells (PBMCs) were obtained from a healthy donor (C-12907, Promocell). The PBMCs in mononuclear cell medium (C-28030, Promocell) were incubated with 10 μg/ml of human serum exosomes in serum-free DMEM for 3-24 h. The human serum exosomes were obtained with 12 patients per disease group. At the end of the experiments, PBMCs were centrifuged, and the supernatant was removed. The PBMC pellets were resuspended and lysed in Qiazol reagent (#79306, Qiagen) for RNA extraction and RT-PCR experiments.

Mouse RAW264.7 macrophages (#TIB-71, ATCC) were cultured in Dulbecco's modified Eagle's medium (DMEM) (#11965-084, ThermoFisher) with 10% fetal bovine serum (FBS) (#10437-028, Life Technologies) and 1% penicillin/streptomycin/glutamine (P/S/G) (#10378-016, Life Technologies) mixture. The macrophages were serum-starved overnight, followed by exposure to fatty acid-free bovine serum albumin (BSA) (A7030, Sigma), sodium palmitate (P9767, Sigma), or lipopolysaccharide (LPS) (L5418, Sigma) for 2 h, and then the addition of elafin (10-1000 ng/ml) for additional 6 h. The cell-conditioned media were used for mouse TNFα ELISA (DY410, R&D Systems).

Quantitative real-time reverse transcription-polymerase chain reaction (RT-PCR) and miRNA PCR array. Total RNA was isolated by RNeasy mini kit (#74104, Qiagen) and reverse transcribed into cDNA by a high-capacity cDNA RT kit (#4368814, ThermoFisher). Quantitative PCR reactions were run with Fast Universal PCR master mix (#4352042, ThermoFisher) in a Bio-Rad CFX384 using cataloged primers (ThermoFisher) for human elafin (Hs00160066_m1), human leptin (Hs00174877_m1), mouse Cd36 (Mm00432403_m1), mouse adiponectin (Mm00456425_m1), mouse leptin (Mm00434759_m1), and mouse Tnf (Mm00443258_m1). Relative mRNA quantification was performed by comparing test groups and control group, after normalization with endogenous control gene human 18S (Hs99999901_s1) or mouse Gapdh (Mm99999915_g1) [68].

For serum exosomal miRNA PCR array determination, serum exosomal total RNA was isolated with RNeasy mini kit (#74104, Qiagen) and reverse transcribed into cDNA by a miScript II RT kit (218161, Qiagen). PCR reactions were run with miScript SYBR Green PCR kit (218073, Qiagen) in a mouse-specific Serum and Plasma miScript miRNA PCR array (MIMM-106ZE-4, Qiagen), which detected 84 miRNAs. Relative miRNA quantification was performed by comparing test groups and control group, after normalization with housekeeping miRNAs.

For additional miRNA determination, RNA was reverse transcribed into cDNA by a miRCURY LNA RT Kit (339340, Qiagen). Quantitative PCR reactions were run with miRCURY LNA SYBR Green PCR kit (339345, Qiagen) in a Bio-rad CFX 384 using miRCURY PCR assays for miR-181b-5p (YP00204530), miR-210-3p (YP00204333), miR-219-5p (YP00204780), and RNU1A1 (YP00203909) from Qiagen Company.

The fold changes are expressed as 2ΔΔCt. Fold-change values greater than one indicate a positive- or an up- regulation, and the fold-regulation is equal to the fold-change. Fold-change values less than one indicate a nega- tive or down-regulation, and the fold-regulation is the negative inverse of the fold-change.

Power analysis. We performed power analyses once we began the experiments. For human serum experiments, we included at least six patients per group to achieve a statistically significant difference of circulating elafin between the non-diabetic group and the prediabetic group (9.6 vs. 10.8 ng/ml) with SD=0.5, alpha=0.05, and power=0.8. For animal studies, we included eight mice per group to achieve a statistically significant difference in fat mass between HFD-treated mice and RD-treated mice (3.18% vs. 0.53%) with SD=1.4%, alpha=0.05, and power=0.8. We did not perform power analysis for cell culture experiments but followed the common prac- tice of performing in vitro experiments three times independently. ELISA and real-time RT-PCR experiments were performed in duplicate.

Statistical analysis. Investigators, except Hon Wai Koon, were blinded to the group allocation. For cell culture experiments, we pooled data from multiple experiments. Bar graphs and scatter plots were made using Microsoft Excel. Equations and R2 values in scatter plots were generated by Microsoft Excel. Results were expressed as mean+/−SEM. Unpaired Student's t-tests were used for two-group comparisons of continuous data (GraphPad QuickCalcs) online. One-way ANOVAs with Tukey Honestly Significant Difference post-hoc tests were used for multiple-group comparisons (Statpages) online. The odds ratio was calculated (Medcalc) online. Significant p values are shown in each figure.

Results

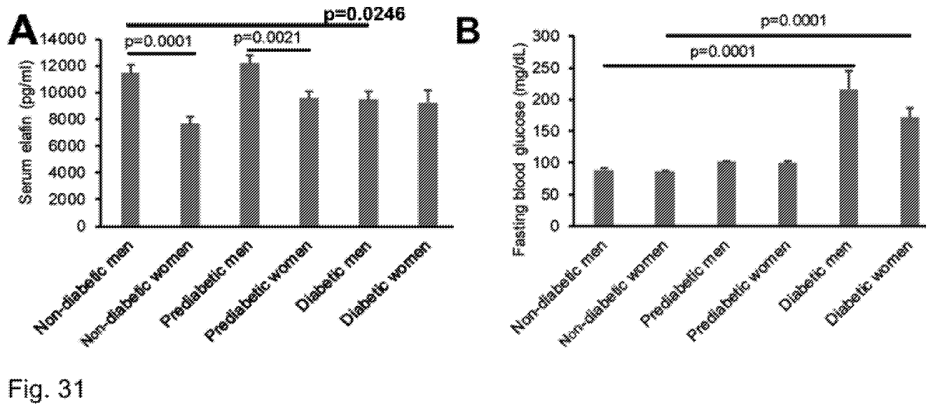

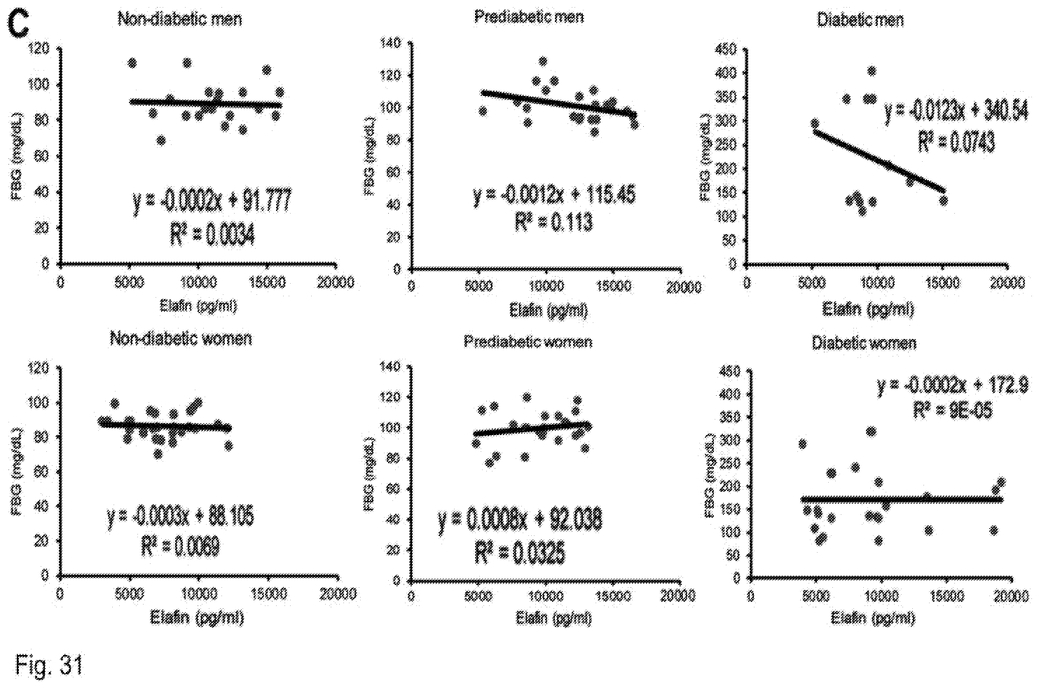

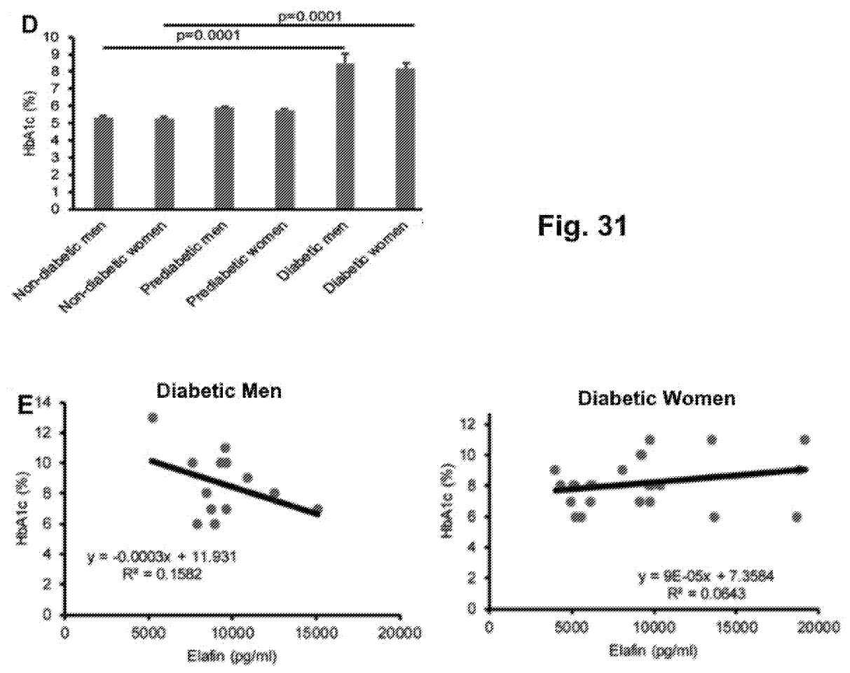

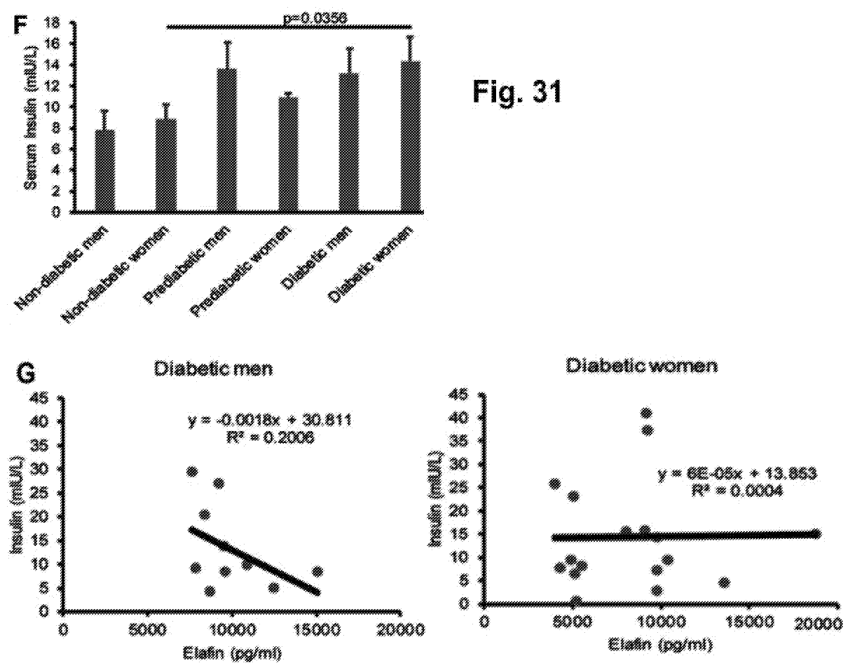

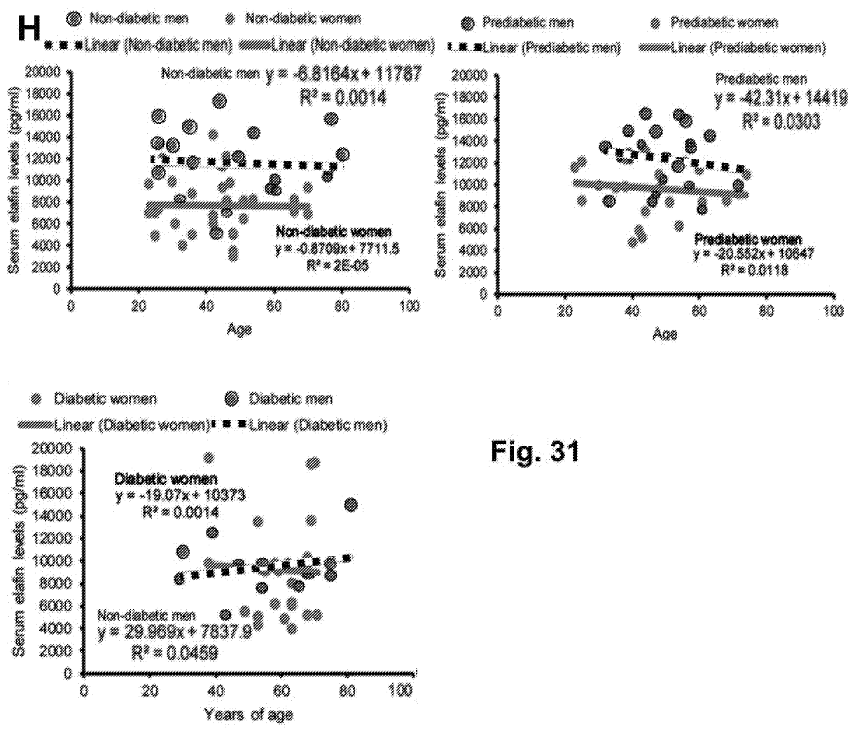

Circulating elafin levels are inversely correlated with fasting blood glucose and HbA1c levels in men with T2DM. To determine the association between circulating elafin protein and diabetes, we measured serum elafin levels of 53 patients without prediabetes/diabetes, 48 patients with prediabetes, and 38 patients with (T2DM). The baseline characteristics of this cohort are shown in Table 51. Among non-diabetic and prediabetic groups, men have significantly higher circulating elafin levels than women (FIG. 31A). Men with T2DM have significantly lower circulating elafin levels than men without diabetes (FIG. 31A). Women with T2DM have relatively low circulating elafin levels, similar to women without prediabetes/diabetes and women with prediabetes (FIG. 31A). All patients with T2DM have significantly higher fasting blood glucose and hemoglobin A1c (HbA1c) levels than non-diabetic and prediabetic patients (FIG. 31B,D).

Women with T2DM also have significantly higher fasting blood insulin levels than women without pre-diabetes/diabetes (FIG. 31F). Men with T2DM and prediabetes also have higher blood insulin levels than men without diabetes, but the differences were statistically insignificant (FIG. 31F). Serum elafin levels are inversely correlated with fasting blood glucose, HbA1c, and insulin levels in men with T2DM, but not women with T2DM (FIG. 31C,E,G). However, circulating elafin levels are independent of age or body mass index (BMI) of all patients (FIG. 31H and S1A-B). These findings suggest that elafin may reduce the severity of diabetes in men, leading us to further pursue this direction with animal models.

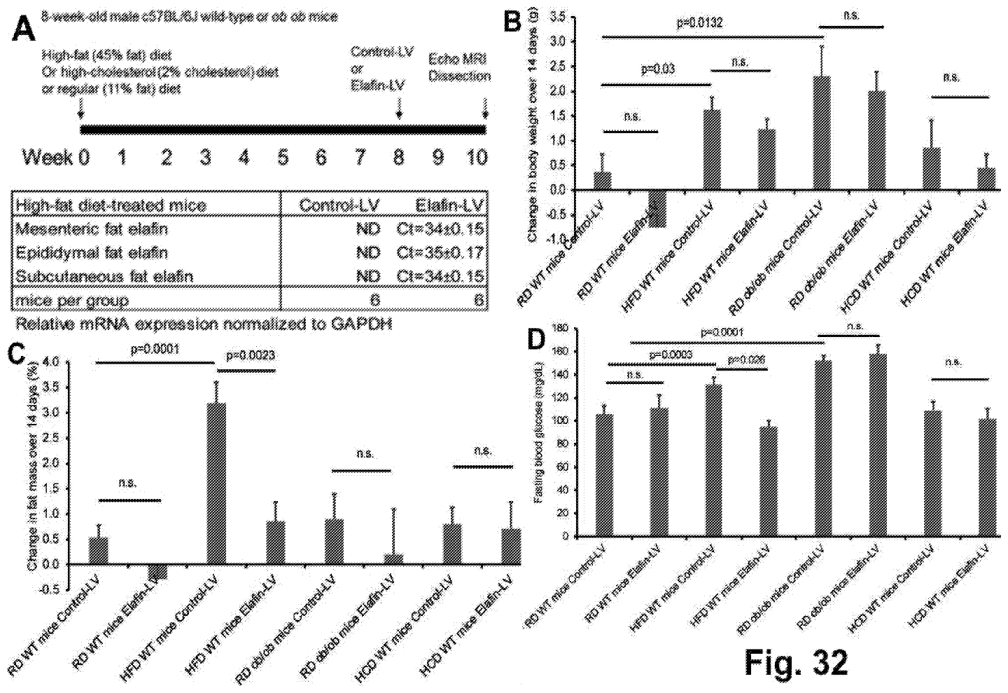

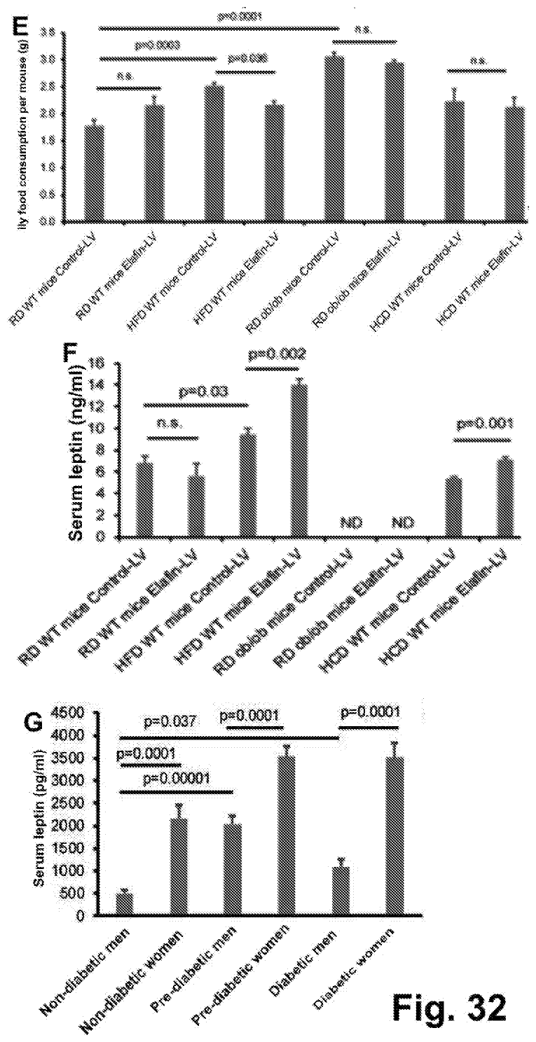

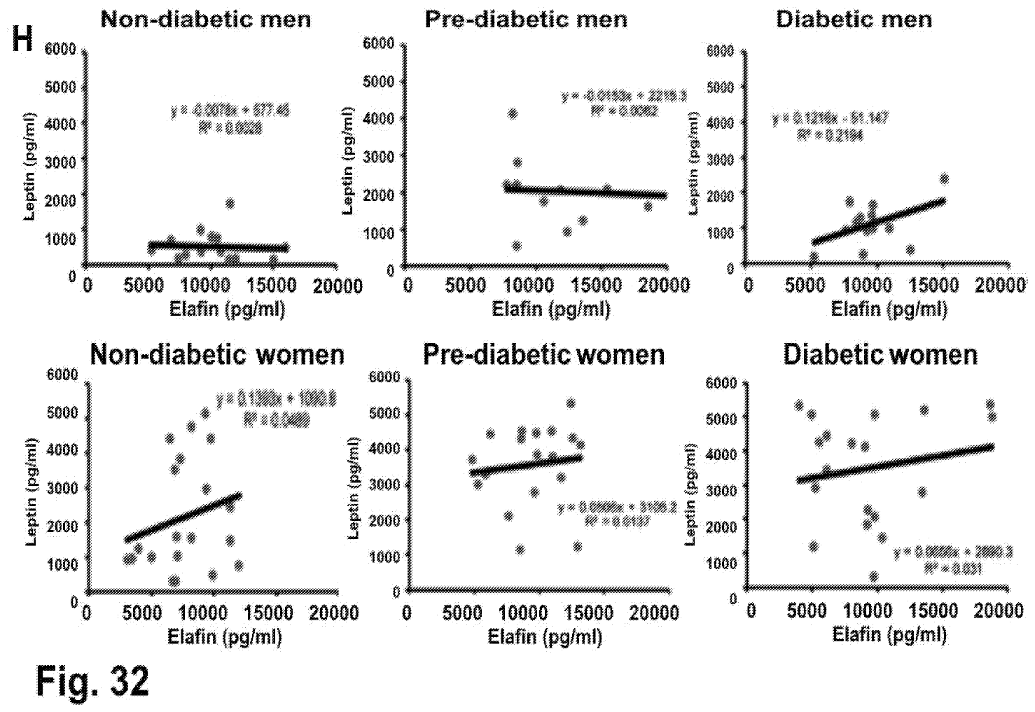

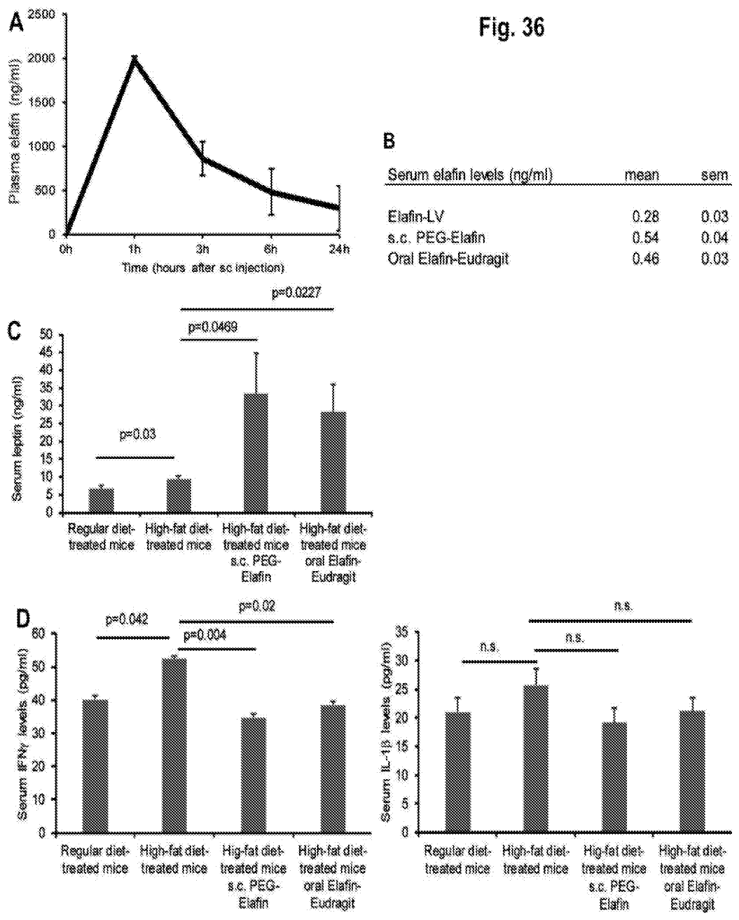

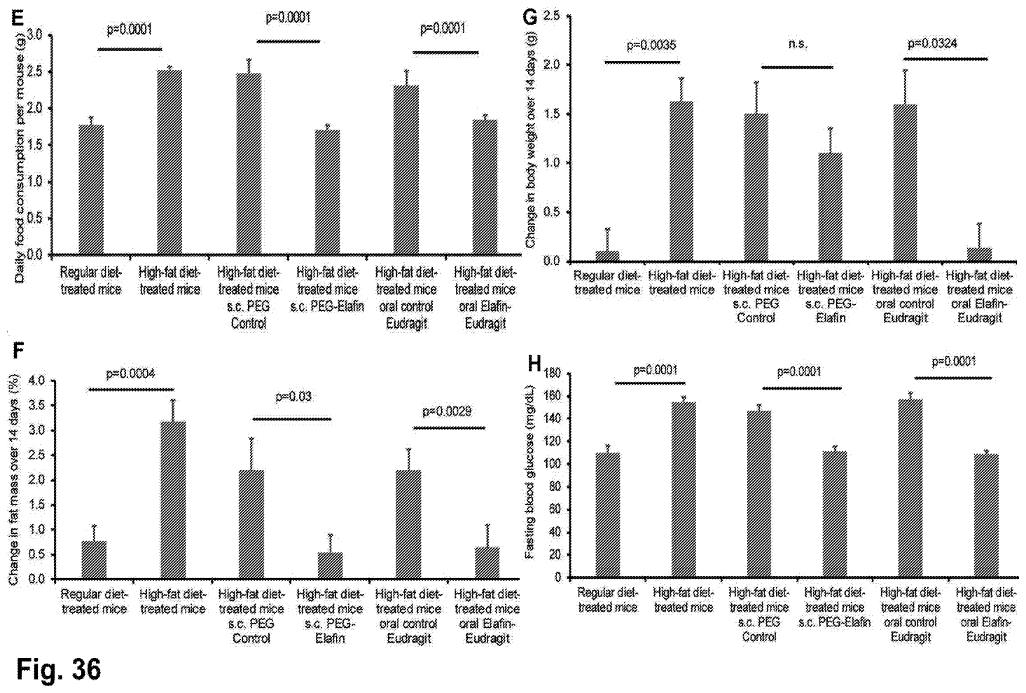

Lentiviral elafin overexpression reduced high-fat diet-induced obesity, hyperglycemia, and hyperphagia in male mice. To evaluate the physiological effects of elafin in diet-induced obesity, hyperglycemia, or hypercholesterolemia, 8-week-old male c57BL/6J wild-type mice received 8-week regular diet (RD), high-fat diet (HFD), or low-fat high-cholesterol diet (HCD) treatment, followed by intravenous injection with either control lentivirus (control-LV) or elafin-expressing lentivirus (elafin-LV)5. Measurements of the various parameters were performed 2 weeks after lentiviral infection (FIG. 32A, upper panel). Mice do not have an elafin gene; therefore, the elafin mRNA signal was undetectable in mice infected with control-LV (FIG. 32A, lower panel). However, the elafin mRNA signal was positive in adipose tissues of elafin-overexpressing mice (elafin-LV group) only (FIG. 32A, lower panel). Elafin protein was detected in the sera of elafin-expressing mice only, i.e., 0.28 ±0.03 ng/ml (mean±sem) (FIG. 36B).

RD-treated male mice had normal fasting blood glucose levels (106±7 mg/dL), which were comparable to the findings of other studies [19,20]. The elafin-mediated reduction of body weight gain and fat mass gain in RD-treated mice was statistically insignificant (FIG. 32B,C). Elafin overexpression did not affect food consumption, fasting blood glucose levels, and serum leptin (appetite-controlling hormone) levels in the RD-treated male mice (FIG. 32D-F).

HFD-treated male mice displayed prediabetic phenotypes with significantly higher body weight, fat mass, fasting blood glucose levels, and food consumption than RD-treated male mice (FIG. 32B-E)[5]. The fasting blood glucose (FBG) levels in our HFD-treated male mice were 131±7 mg/dL, which is regarded as prediabetic [21]. As this study sought to determine the therapeutic effects of elafin against diabetes, female mice were not included because HFD-treated female mice do not develop hyperglycemia [22]. Elafin overexpression significantly reduced fat mass gain (by 2.3%), fasting blood glucose levels (by 27%), and food consumption (by 13.8%) in HFD-treated, but not in HCD-treated male mice within 14 days (FIG. 32C-E). In an oral glucose tolerance test (OGTT), glucose feeding elevated blood glucose levels in RD- and HFD-treated mice (FIG. S2A) [5]. Elafin overexpression modestly reduced blood glucose levels in the HFD-treated mice, but the difference was statistically insignificant (FIG. S2A).

Consistent with previous studies [23-25], HFD treatment increased circulating insulin and total cholesterol, but not free fatty acid and adiponectin levels in mice (FIG. S2B-E). HCD treatment increased circulating total cholesterol levels without affecting fat mass and fasting blood glucose levels in mice (FIG. S2C, FIG. 32C-D). Elafin overexpression did not significantly affect body weight, insulin, total cholesterol, free fatty acid, and adiponectin levels in the HFD-treated and HCD-treated mice (FIG. 32B, FIG. S2B-E).

Elafin reduced food consumption via increased leptin expression in mesenteric fat of HFD-treated male mice. To explain the decreased food consumption in elafin-overexpressing mice, we measured leptin levels in blood and leptin mRNA expression in fat tissues as leptin, an adipose-derived hormone, reduces food intake [26]. HFD-treated mice, but not HCD-treated mice, have significantly higher circulating leptin levels than RD-treated mice (FIG. 32F). Elafin-overexpressing mice had higher circulating leptin levels than control lentivirus-expressing mice in both HFD and HCD treatment (FIG. 32F). Leptin was not detectable in the sera of ob/ob mice as they are leptin-deficient (FIG. 32F). We included leptin-deficient ob/ob male mice to confirm the role of leptin in this phenotype. The ob/ob mice had significantly higher body weight gain, fasting blood glucose levels, and food consumption than wild-type mice, which were not affected by elafin overexpression (FIG. 32B-E).

Similarly, patients with prediabetes and T2DM have significantly higher circulating leptin levels than patients without diabetes (FIG. 32G). Women also have significantly higher circulating leptin levels than men among all groups (FIG. 32G). Circulating leptin levels are positively correlated with circulating elafin levels in men with T2DM, but not in women with T2DM (FIG. 32H).

The HFD treatment increased long-chain fatty acid transporter and scavenger receptor Cd36 and leptin mRNA expression in mesenteric and epididymal fat in mice, compared to regular diet treatment (FIG. S2E). Elafin overexpression significantly increased leptin mRNA expression in mesenteric fat only (FIG. S3A) and decreased fat receptor Cd36 mRNA expression in both mesenteric and epididymal fat in the HFD-treated mice (FIG. S3A).

We considered the possible involvement of intestinal microbiota in the actions of elafin. Cecal microbiota transplantation from the elafin-overexpressing donor mice did not significantly affect the fat mass, body weight, fasting blood glucose levels, and food consumption in the HFD-treated recipient mice (FIG. S3B). Elafin over-expression does not appear to affect the intestinal environment.

Immune cells mediate the protective effects of elafin in HFD-treated Rag−/− male mice. Since elafin is an anti-inflammatory protein [14], we utilized multiplex ELISA to profile serum cytokines of patients and evaluated the association between circulating elafin and inflammation in patients (FIG. S4A). Serum IFNγ levels are inversely correlated with serum elafin levels in men with T2DM, but not women with T2DM (FIG. S4C-D). Serum IL-1β levels are not associated with serum elafin levels in patients with T2DM (FIG. S4A).

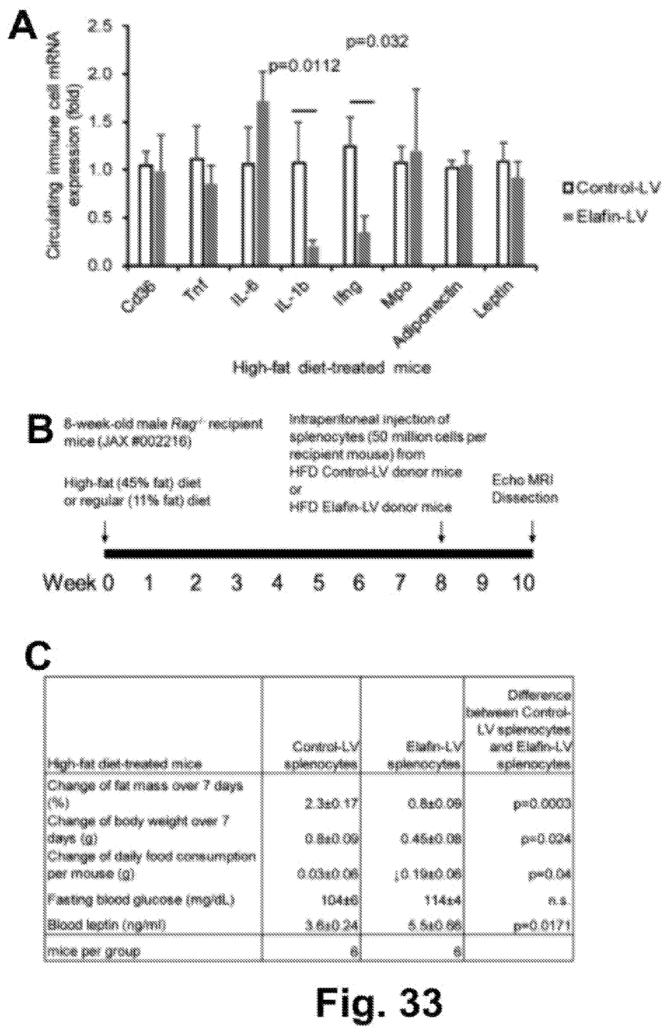

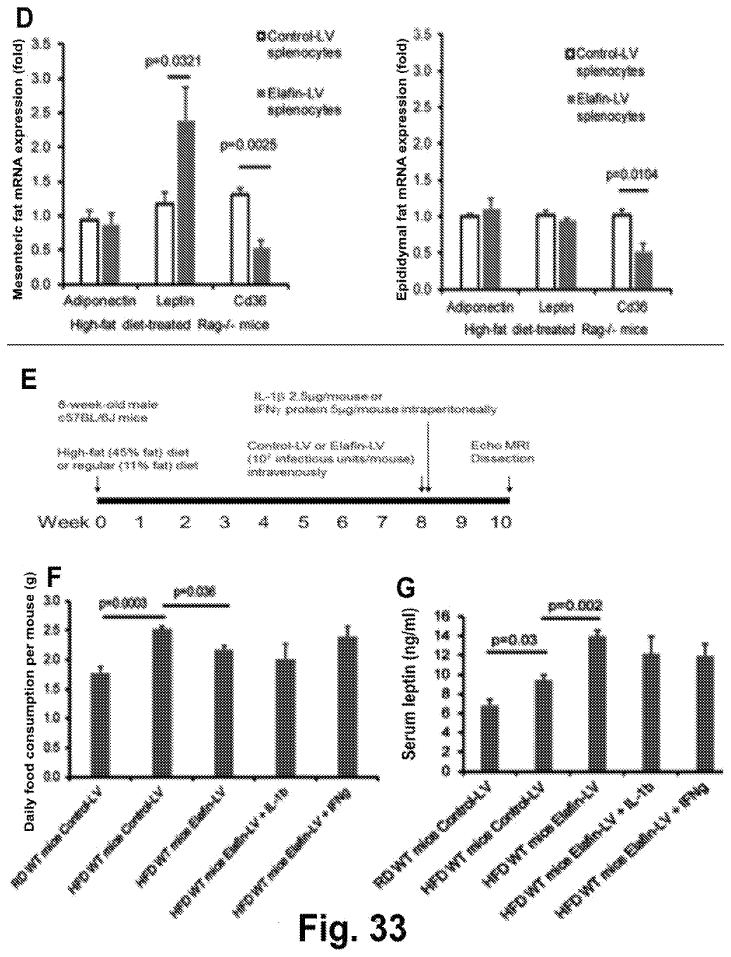

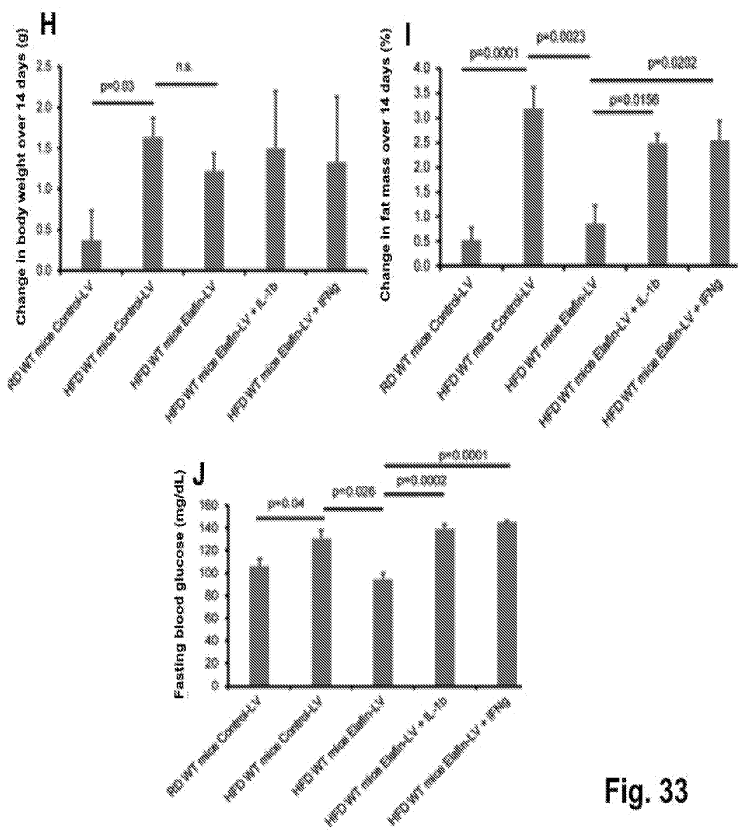

We evaluated the cytokine and hormone mRNA expression in circulating white blood cells. HFD-treated control-LV and elafin-LV mice had similar myeloperoxidase/Mpo (neutrophil), and adiponectin and leptin (hormone) mRNA expression in their circulating white blood cells (FIG. 33A). Interestingly, elafin overexpression significantly reduced interleukin-1beta (IL-1β) and interferon-gamma (IFNγ), but not tumor necrosis factor (Tnf) and interleukin-6 (Il-6), mRNA expression in circulating white blood cells of the HFD-treated mice (FIG. 33A), indicating the involvement of circulating immune cells and cytokines in the elafin-mediated anti-diabetic effects.

We transplanted splenocytes from HFD-treated control-LV or elafin-LV mice to HFD-treated Rag−/− recipient mice, which are mature B and T lymphocytes deficient (FIG. 33B) [27]. This approach enabled us to investigate the effects of elafin-conditioned immune cells without using direct elafin overexpression in the HFD-treated recipient mice. After the injection of splenocytes from HFD-treated control-LV male donor mice, the HFD-treated Rag−/− male recipient mice continued to increase fat mass and body weight (FIG. 33C). Recipient mice of HFD- treated elafin-overexpressing mouse (Elafin-LV) splenocytes showed significantly increased serum leptin levels with decreased fat mass gain (by 1.5%), body weight gain (by 44%), and food consumption (by 0.19 g), compared to those injected with control splenocytes (FIG. 33C). Fasting blood glucose levels were not significantly affected by splenocyte transplantation (FIG. 33C) because the 129 strain recipient mice were resistant to the development of insulin resistance [28]. Similar to the elafin-overexpressing donor mice, recipient mice of elafin-overexpressing mouse splenocytes had increased leptin mRNA expression in mesenteric fat only and reduced Cd36 mRNA expression in the mesenteric fat and epididymal fat (FIG. 33D).

IFNγ is involved in the elafin-dependent regulation of food consumption in HFD-treated male mice. To determine the involvement of immune cell-derived IL-1β and IFNγ in the elafin-mediated effects, we injected IL-1β and IFNγ protein to the HFD-treated elafin-overexpressing mice intraperitoneally (FIG. 33E). IFNγ, but not IL-1β, moderately increased food consumption in the elafin-overexpressing mice, suggesting that elafin-dependent IFNγ expression may affect leptin sensitivity (FIG. 33F). Injection of either cytokine reversed the elafin-mediated inhibition of fat mass and fasting blood glucose levels, but not serum leptin levels and body weight gain of the HFD-treated elafin-overexpressing mice (FIG. 33G-J).

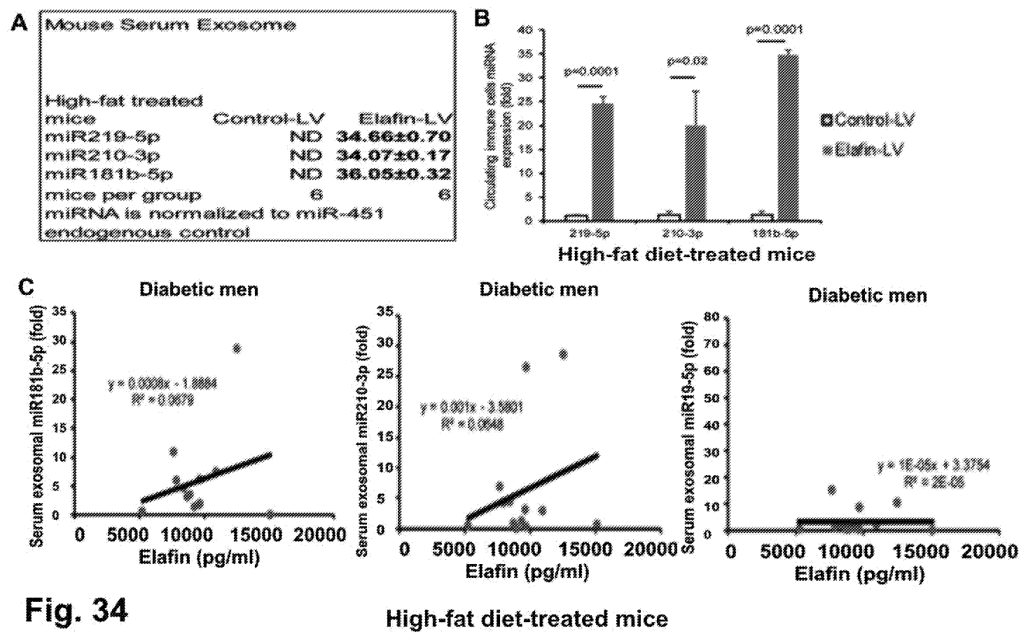

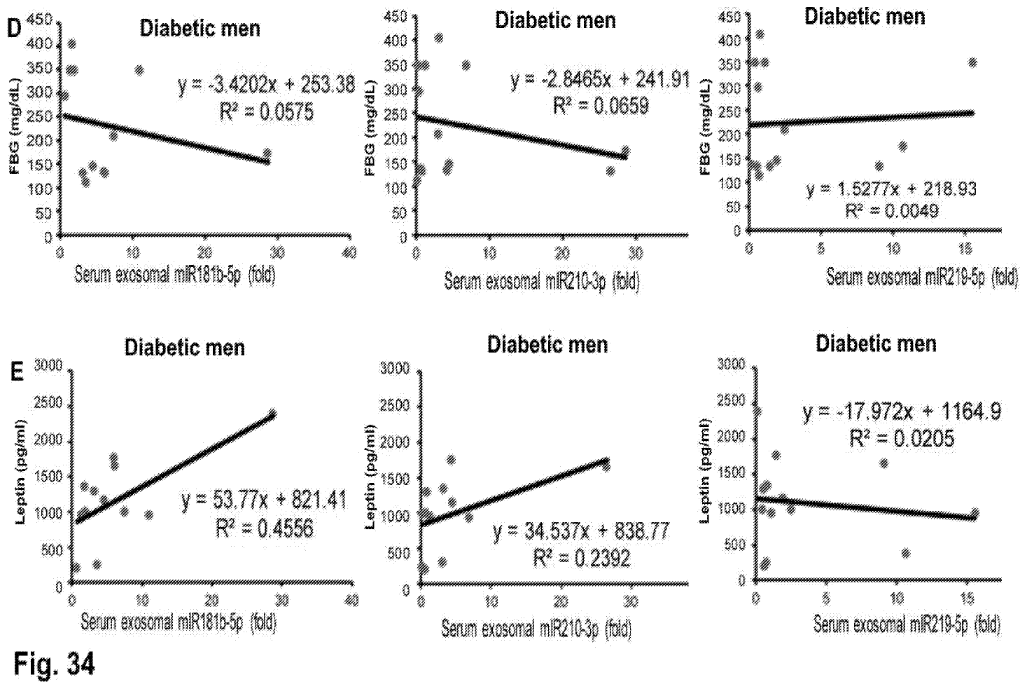

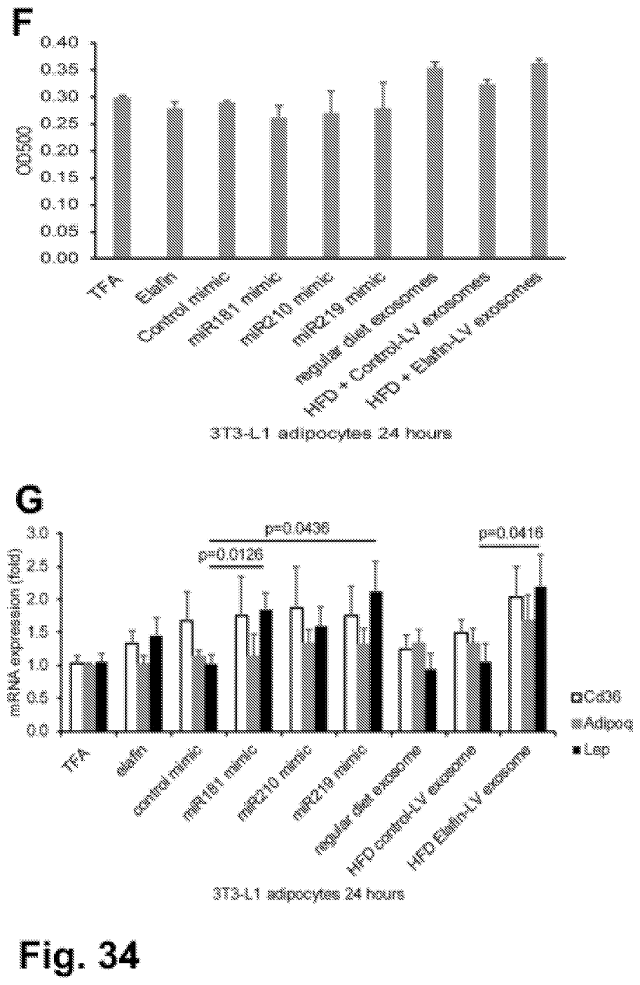

Circulating exosomes and immune cells shared similar miRNA expression profiles in elafin-overexpressing HFD-treated male mice. In addition to cytokine expression, immune cells also regulate immune responses via exosome secretion [29,30]. Exosomes are crucial in disease processes of diabetes by carrying miRNAs to target organs [31-34]. To determine whether elafin influences circulating exosomal miRNA expression, we used a PCR array to profile miRNAs in mouse serum exosomes. Signals of miR219-5p, miR210-3p, and miR181b-5p were detectable in the serum exosomes of HFD-treated, elafin-overexpressing mice only (FIG. 34A). Similarly, elafin overexpression significantly increased miR181b-5p, miR210-3p, and miR219-5p expression in circulating white blood cells, but not in mesenteric and epididymal fat tissues of HFD-treated male mice (FIG. 34B and S3C), suggesting that these three serum exosomal miRNAs are derived from circulating immune cells.

Circulating exosomal miR181b-5p and miR219-5p expression was correlated with blood glucose and leptin levels in men with T2DM. The serum exosomal miR181b-5p, miR210-3p, and miR219-5p expression in patients are shown in FIG. S1C, S1E, and S1G. The associations between serum exosomal miR181-5p, miR210-3p, and miR219-5p expression and circulating elafin levels in patients are shown in FIG. S1D, S1F, and S1H.

Interestingly, serum exosomal miR181b-5p and miR210-3p, but not miR219-5p, expression was positively correlated with circulating elafin levels in men with T2DM (FIG. 34C). In these men with T2DM, serum exosomal miR181b-5p and miR210-3p expression were negatively correlated with fasting blood glucose levels but positively correlated with circulating leptin levels (FIG. 34D-E). Circulating exosomal miR219-5p expression was not associated with blood glucose and leptin levels in men with T2DM (FIG. 34D-E).

Elafin-dependent serum exosomal miR181b-5p and miR219-5p induced leptin mRNA expression in adipocytes. Differentiation of preadipocytes to adipocytes is characteristic of lipid accumulation, adiponectin (adipokine) production, and increased CD36 expression [35-37]. We incubated mouse adipocytes with elafin, miRNA mimics, and exosomes. Exposure to elafin protein, mouse serum exosomes (from all groups), and miRNA mimics (181-5p, 219-5p, and 210-3p) affected neither lipid accumulation (FIG. 34F) nor Cd36 and adiponectin mRNA expression (FIG. 34G) in adipocytes, suggesting that elafin does not affect adipocyte differentiation in vitro.

Serum exosomes from elafin-overexpressing mice, miR181b-5p mimic, and miR219-5p mimic, but not miR210-3p mimic and elafin protein, significantly increased leptin mRNA expression in mouse 3T3-L1 adipocytes (FIG. 34G). Similarly, transfection of either miR181-5p mimic or miR219-5p mimic significantly increased leptin secretion in primary human mesenteric fat adipocytes (FIG. S3D). Elafin, via serum exosomal miR181b-5p and miR219-5p, induces leptin expression in adipocytes indirectly. Therefore, we selected miR181b-5p and miR219-5p for functional validation in vivo.

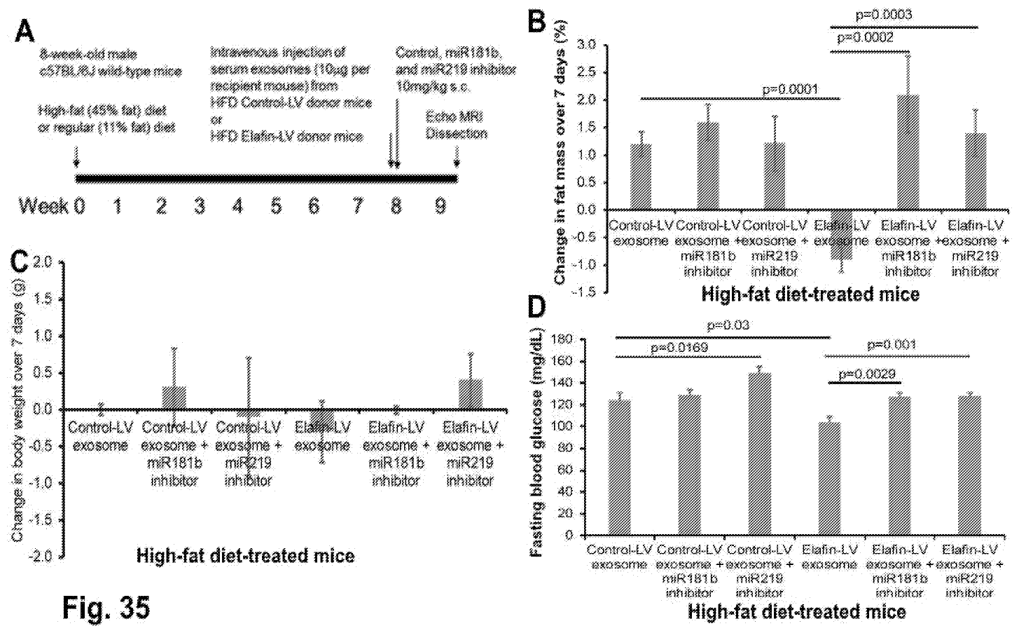

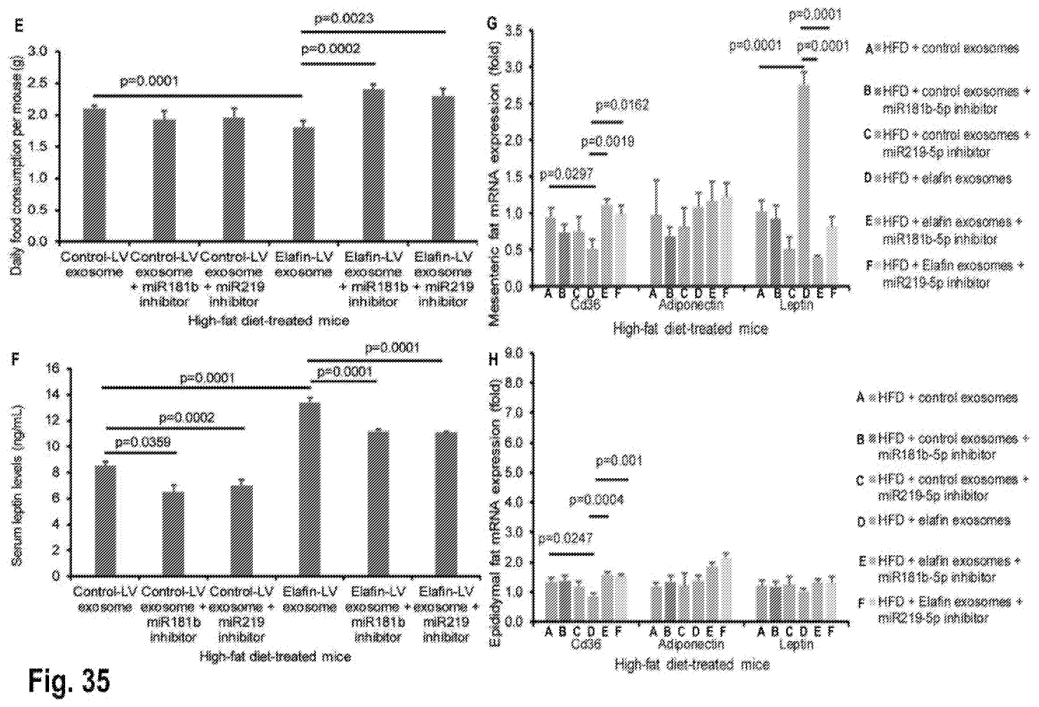

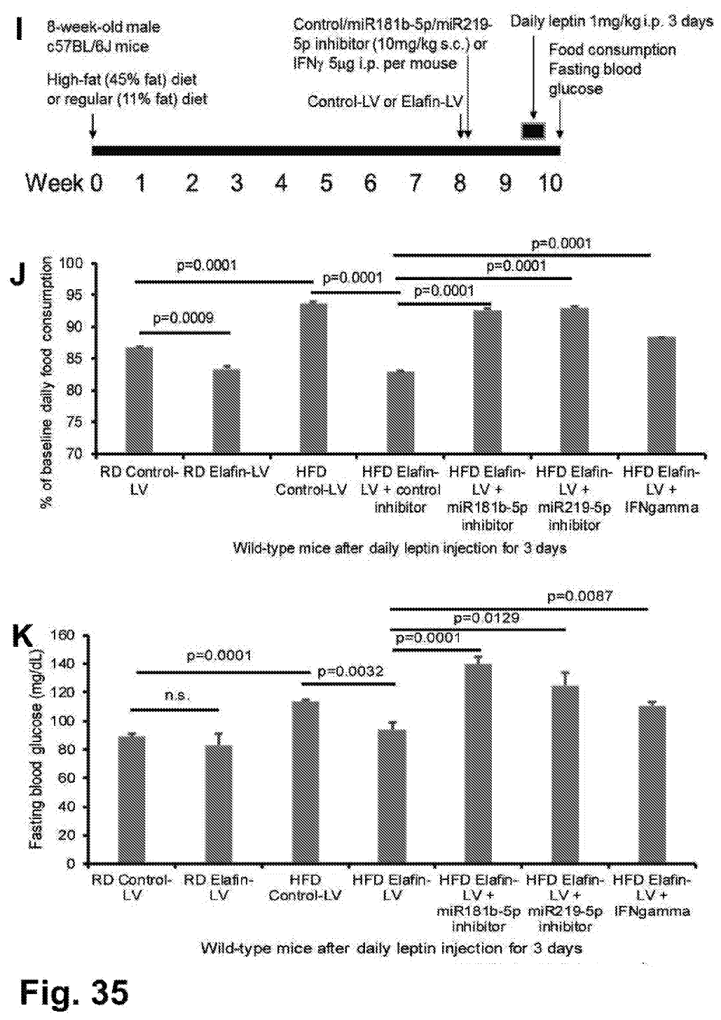

Elafin mediates anti-obesity and anti-diabetic effects via serum exosomal miR181b-5p and miR219-5p in the HFD-treated male mice. To determine the role of elafin-conditioned circulating exosomes in regulating food consumption, obesity, and hyperglycemia, we transplanted serum exosomes to HFD-treated male recipient mice intravenously (FIG. 35A). Injection of serum exosomes did not significantly affect the body weight in the recipient mice (FIG. 35B). Recipient mice of elafin-overexpressing mouse exosomes had significantly reduced fat mass gain (by 2.1%), fasting blood glucose levels (by 16%), and food consumption (by 14%), compared to those injected with control exosomes (FIG. 35C-E). Injection of elafin-LV serum exosomes did not alter circulating levels of total cholesterol, insulin, free fatty acid, and adiponectin in the recipient mice (FIG. 52A-D).

Transplantation of elafin-LV serum exosomes significantly increased serum leptin levels and mesenteric fat leptin mRNA expression in the recipient mice (FIG. 35F-G). The same recipient group also had reduced Cd36 mRNA expression in epididymal and mesenteric fat (FIG. 35G,H). All of these elafin-dependent effects were reversed by miR181b-5p and miR219-5p inhibitors (FIG. 35F-H). Therefore, elafin induces leptin expression and reduces food consumption, obesity, and hyperglycemia via serum exosomal miR181b-5p and miR219-5p.

Elafin reduces circulating IFNγ levels via serum exosomal miR181b-5p in HFD-treated male mice. In addition, we determined whether elafin-conditioned exosomes affect the circulating levels of cytokines in the HFD-treated mice. HFD treatment significantly increased circulating levels of IFNγ in the mice, which were reduced by elafin-LV serum exosome treatment (FIG. S4E). Interestingly, miR181b-5p inhibitor, but not miR219-5p inhibitor, reversed the elafin-dependent suppression of IFNγ levels (FIG. S4E). Neither HFD nor elafin-LV treatment affected serum IL-1β levels in the mice (FIG. S4E). HFD-treatment also mildly increased (statistically insignificant) the circulating levels of other detected cytokines (such as IL-1β, GM-CSF, IL-12p70, IL-2, IL-3, IL-4, IL-17A, MIP-1α, MIP-1β, and TNFα), but the levels of these ten cytokines were not affected by elafin-LV exosome treatment (FIG. S4B). Thus, elafin-dependent exosomes reduced circulating IFNγ levels via miR181b-5p.

Elafin enhanced leptin sensitivity in the HFD-treated male mice. In addition to elafin-mediated leptin expression, we determined whether elafin affects leptin sensitivity in mice. The HFD-treated male mice received daily intraperitoneal leptin injection for 3 days, followed by measurement of food consumption and fasting blood glucose levels (FIG. 351). After 3 days of leptin injection, RD-treated elafin-LV group showed mod- erately reduced food consumption, but not fasting blood glucose levels (FIG. 35J,K). HFD-treated mice showed impaired leptin sensitivity as represented by high food consumption and fasting blood glucose levels after leptin treatment (FIG. 35J,K). Leptin injection further reduced food consumption in HFD-treated elafin-overexpressing mice (FIG. 35J). Fasting blood glucose was reduced in HFD-treated elafin-overexpressing mice, but this decrease was not affected by leptin injection (FIG. 35K). Elafin-mediated decrease of food consumption and fasting blood glucose levels were reversed by subcutaneous injection of miR181b-5p inhibitor, miR219-5p inhibitor, and IFNγ (FIG. 35J,K). Therefore, elafin restores leptin sensitivity in the HFD-treated male mice via increased expression of miR181b-5p and miR219-5p and inhibition of IFNγ.

Elafin inhibited liver steatosis in the HFD-treated male mice. HFD treatment caused liver steatosis in mice (FIG. S5A). Elafin overexpression did not affect the normal liver histology in RD-treated male mice (FIG. S5A and B). Elafin overexpression inhibited liver steatosis in the HFD-treated male mice (FIG. S5A and B) [5,38], which was reversed by injection of IL-1β or IFNγ protein (FIG. S5A and B). Similar to elafin over- expression, transplantation of splenocytes from elafin-overexpressing mice or circulating exosomes from elafin- overexpressing mice also inhibited liver steatosis (FIG. S5C-F). The quantitative changes of steatosis were reflected by non-alcoholic fatty liver steatosis subscore (FIG. S5B, D, and F).

HFD treatment significantly increased fat receptor Cd36 mRNA expression in the liver, which was inhibited by either lentiviral elafin overexpression, transplantation of splenocytes from elafin-overexpressing mice, or transplantation of circulating exosomes from elafin-overexpressing mice (FIG. 56A-C). The elafin-dependent exosomal inhibition of steatosis and hepatic Cd36 mRNA expression was reversed by miR181b-5p and miR219-5p inhibitors (FIG. S5E-F and S6C).

RD-treated leptin-deficient ob/ob male mice and HCD-treated male mice developed liver steatosis (FIG. S6D-E) [39,40]. Elafin overexpression was ineffective in inhibiting liver steatosis in ob/ob mice (FIG. S6D). The results suggest that elafin inhibits liver steatosis via leptin expression. Elafin overexpression also failed to inhibit liver steatosis and hepatic Cd36 mRNA expression in the HCD-treated mice (FIG. S6A and S6E).

Subcutaneous PEG-Elafin and oral Elafin-Eudragit formulation inhibited obesity, hyperglycemia, and liver steatosis in the HFD-treated male mice. The subcutaneous injection of natural elafin to RD-treated male mice showed a short half-life (3 h) in circulation (FIG. 36A). We generated PEGylated elafin for subcutaneous injection and elafin-Eudragit formulation for oral administration. Both subcutaneous and oral administration of the modified elafin formulations produced comparable serum elafin levels as lentiviral elafin overexpression in the HFD-treated male mice (FIG. 36B). Both elafin delivery approaches also significantly increased circulating leptin levels (FIG. 36C) and significantly reduced circulating IFNγ levels, food consumption, fat mass gain, fasting blood glucose levels, and liver steatosis of the HFD-treated male mice (FIG. 36D-H and S6F). Also, oral elafin-Eudragit administration, but not subcutaneous PEG-Elafin injection, reduced body weight gain in the HFD-treated male mice (FIG. 36G).

Discussion

This report is the first to demonstrate the reduced circulating elafin levels in men with T2DM (FIG. 31A). The reason behind the gender difference in serum elafin levels in non-diabetic and prediabetic patients is unknown. Interestingly, a previous study demonstrated that female sex hormone (estradiol) suppressed elafin secretion in human vaginal epithelial cells [41]. Another study showed that estrogen receptor-positive breast cancer tumors have lower elafin mRNA expression than estrogen receptor-negative counterparts [42]. Estrogen, via estrogen receptor, inhibits elafin expression in human cells that may lead to reduced circulating elafin levels in women without prediabetes/diabetes and women with prediabetes. On the other hand, the low serum elafin levels in patients with T2DM are gender independent (FIG. 31A). We speculate that the inhibitory effect of estrogen becomes trivial when the serum levels in women with diabetes are already low.

Serum elafin levels are not correlated with BMI in men with T2DM (FIG. SIB), possibly because BMI is not an ideal indicator for obesity among patients with abnormal blood glucose levels [43]. In this study, fat mass and calorie intake were not determined among patients with T2DM. Interestingly, elafin overexpression and elafin-LV exosome injection caused a statistically significant reduction of fat mass and an insignificant reduction in body weight of mice (FIGS. 30B,C, 33B,C). The change of fat mass (less than 3.5% range) had a minor impact on the body weight because HFD-treated male mice have 20% fat mass and 80% lean mass, while RD-treated male mice have 10% fat mass and 90% lean mass.

This study is the first to illustrate the elafin-dependent regulation of food consumption via the immune system in mice (FIG. 33). We speculate that the decreased circulating elafin levels in men with T2DM are caused by reduced elafin expression in immune cells, as exposure to serum exosomes from patients with T2DM inhibited elafin mRNA expression in PBMCs (FIG. S4F). PBMCs consist of a significant portion of T and B lymphocytes. Exposure to serum exosomes from patients with T2DM and high serum elafin levels significantly increased miR181b-5p and miR210-3p expression in PBMCs (FIG. S4G). This finding indicates that circulating immune cells produce these elafin-regulated miRNAs and supports the positive correlation between serum elafin levels and serum exosomal miR181b-5p and miR210-3p in men with T2DM (FIG. 34C).

Elafin overexpression led to a similar pattern of increased miR181b-5p, miR210-3p, and miR219-5p expression in both circulating immune cells and serum exosomes in the HFD-treated mice (FIG. 34A,B), while transplantation of these elafin-conditioned splenocytes and serum exosomes were equally effective in reducing food intake in HFD-treated male recipient mice (FIGS. 33, 35). Both mouse studies suggest that these appetite-regulating serum exosomal miRNAs are derived from splenocyte-derived immune cells. Lymphocytes are capable of secreting exosomal miRNAs that modulate diabetes development [44]. The elafin-dependent regulation of food consumption in the HFD-treated mice should involve T- and B-lymphocytes because they constitute a majority of splenocytes and have a long lifespan (several weeks). Splenic dendritic cells are a relative minority of splenocytes and last only a few days.

The disruption of elafin-dependent anti-obesity, anti-diabetic, and hepatoprotective effects in leptin-deficient ob/ob male mice reflect their absolute dependency on leptin (FIG. 32B,E and S5D). Humans and mice have differences in gene expression and physiological responses. For example, elafin-dependent miR181b-5p and miR210-3p are associated with leptin in men with T2DM (FIG. 34E), while elafin regulates leptin expression via miR181b-5p and miR219-5p in HFD-treated male mice (FIG. 35). The correlation between serum elafin, cytokines (IFNγ), and exosomal miRNAs (miR181b-5p) in men with T2DM are similarly reflected by the elafin-mediated inhibition of IFNγ expression and promotion of miR181b-5p expression in HFD-treated male mice (FIGS. 33, 34 and 54A-E).

Elafin overexpression inhibited hyperphagia and promoted leptin-mediated suppression of food consumption in the HFD-treated male mice, which were reversed by IFN-γ injection (FIGS. 33F, 35J). IFNγ deficiency is associated with the improvement of leptin sensitivity. For example, RD-treated IFNγ deficient male mice have reduced food consumption [45], while HFD-treated IFNγ deficient mice have improved insulin sensitivity, reduced adipocyte diameter, and lowered serum leptin levels [46,47]. These findings suggest that elafin improves leptin sensitivity by inhibiting IFNγ expression in HFD-treated male mice.

Although miR181b-5p and miR219-5p are known to regulate glucose homeostasis and obesity respectively [48,49], our study is the first to address how elafin-dependent miR181b-5p and miR219-5p regulate adipose leptin expression (FIG. 34G). Consistent with our finding (FIG. S4E), miR181b also inhibits IFNγ expression in human CD4 Th1 lymphocytes [50]. As HFD-treated mice are leptin resistant [51], elafin-dependent immune-derived serum exosomal miR181b reduces IFNγ mRNA expression, lowers circulating IFNγlevels, and subsequently improves leptin sensitivity (FIGS. 33A,F, 32A, and S6D). At the same time, elafin-dependent miR181b-5p and miR219-5p induce leptin expression in human and mouse adipocytes (FIG. 34G and S3D). These two co-existing complementary pathways enable elafin to restore the leptin pathway and suppress food consumption.

Although elafin inhibits IL-1β expression in the circulating immune cells of HFD-treated mice (FIG. 33A), elafin does not improve leptin sensitivity via IL-1β inhibition because IL-1β injection did not increase food consumption in the elafin-overexpressing mice (FIG. 33F). The exacerbated hyperglycemia in the IL-1β-treated elafin-overexpressing mice may reflect IL-1β-induced insulin resistance in adipocytes (FIG. 33F) [52].

The elafin-dependent mesenteric fat and hepatic CD36 expression are associated with obesity and liver steatosis in mice (FIG. S2E, right panel, S3A, and S6A-C). However, elafin, miR181b-5p mimic, or miR219-5p mimic did not directly alter Cd36 mRNA expression in the adipocytes in vitro (FIG. 32G). We speculate that elafin-dependent CD36 expression is regulated by food consumption of the mice as adipose and hepatic Cd36 mRNA expression varies upon calorie intake [53,54].

As liver steatosis in HFD-treated mice is CD36-dependent [38], the elafin-driven reduction of hepatic Cd36 mRNA expression should mediate the inhibition of liver steatosis (FIG. S6A). Elafin may not affect cholesterol synthesis in the liver because elafin overexpression did not affect hepatic HMG-CoA reductase mRNA expression in HFD- and HCD-treated mice (FIG. S6G). HFD-treatment activated a mixed population of resident Kupffer cells and recruited macrophages [55], as reflected by significantly increased hepatic F4/80 mRNA expression in the HFD-treated mice (FIG. S6H).

Hepatic IL-6 expression is positively correlated with the severity of nonalcoholic steatohepatitis (NASH) in patients [56], while hepatic TNF expression is associated with liver fibrosis among patients with NASH [57]. Consistent with our microscopic observations (FIG. S5-S6), the significantly increased hepatic 116, but not TNF, mRNA expression indicated the presence of hepatic inflammation without fibrosis in the HFD-treated mice (FIG. S6I). The exogenous elafin-regulated inhibition of hepatic F4/80 mRNA expression, along with the modestly reduced hepatic pro-inflammatory cytokine (116) mRNA expression, might reflect the amelioration of hepatic inflammation (FIG. S6I).

Liver enzyme levels may reflect liver injury among patients. There is no correlation between serum elafin levels and liver enzyme levels (ALT, AST, and ALP) among patients with T2DM (FIG. 57A-F). The presence of non-alcoholic fatty liver disease (NAFLD) is independent of serum elafin levels, severity of diabetes, or liver enzyme levels among patients with T2DM (FIG. S7G).

Adipose tissue depots in obese mice consist of more than 50% F4/80+macrophages [58], which are associated with obesity and insulin resistance [59]. A previous mouse study also showed that macrophages, instead of adipocytes, express almost all of the adipose tissue-derived TNFα and a significant part of adipose tissue-derived IL-6 [58]. However, elafin overexpression did not affect F4/80, Tnf, and II-6 mRNA expression in mesenteric, epididymal, and subcutaneous fat of HFD-treated mice (FIG. S3A). Furthermore, elafin did not affect lipopolysaccharide (LPS)—and palmitate-induced TNFα secretion in mouse RAW264.7 macrophages (FIG. S3E). These findings suggest that elafin is unlikely to affect adipose tissue macrophage accumulation and activation in HFD-treated mice.

Given the large proportion of macrophages in the adipose tissues of obese mice [58], elafin overexpression did not affect miR181b-5p, miR210-3p, and miR219-5p expression in mesenteric and epididymal fat tissues of HFD-treated mice (FIG. S3C). This evidence suggests that elafin does not regulate the expression of these miRNAs in the adipose macrophages and adipocytes of HFD-treated mice.

Consistent with the increased leptin mRNA expression in the mesenteric fat of HFD-treated mice (FIG. S2E), leptin mRNA expression in mesenteric fat is positively correlated with the BMI values of patients without diabetes (FIG. S8A-B). Adipose elafin mRNA expression and miR181b-5p, miR210-3p, and miR219-5p expression are independent of the BMI values of these patients (FIG. S8C). Adipose miR181b-5p, miR210-3p, and miR219-5p expression and leptin mRNA expression are not associated with adipose elafin mRNA expression in patients without diabetes, as shown by low R2 values (FIG. S8D-E).

Although systemic injection of elafin (200 mg per subject) was well tolerated in humans [60], formulation opti- mization is necessary to overcome the short half-life of elafin in circulation (FIG. 36A). For example, PEGylation extends the half-life of subcutaneously injected drugs in circulation. Alternatively, Eudragit coating protects drugs from gastric acid inactivation and releases it in the alkaline terminal ileum and colon, which is common in orally-active medications for gastrointestinal and metabolic diseases [61,62]. As both subcutaneous PEG-elafin and oral Elafin-Eudragit formulations mimicked the protective effects of elafin overexpression (FIG. 36), they should be clinically useful for reversing diabetes.

In conclusion, circulating elafin levels are reduced and inversely correlated with hyperglycemia in men with T2DM. As shown by our mouse studies, elafin inhibits hyperglycemia via pleiotropic mechanisms. Elafin over-expression increases leptin sensitivity by suppressing immune cell-derived IFNγ in HFD-treated male mice. Elafin overexpression also induces appetite-lowering leptin expression in mesenteric fat via immune cell-derived exosomal miR181b-5p and miR219-5p expression in the HFD-treated male mice. Leptin-mediated reduced food intake subsequently inhibits obesity, hyperglycemia, and liver steatosis in HFD-treated male mice. Subcutaneous PEG-Elafin injection and oral Elafin-Eudragit formulation administration are also effective against obesity, hyperglycemia, and liver steatosis in HFD-treated male mice. The discoveries of this study provide vital information and tools for improving the management of diabetes.

### REFERENCES

1. Bullard, K. M. et al. MMWR Morb. Mortal. Wkly.Rep. 67, 359-361. (2018).
2. Chen, L., et al. Nat. Rev. Endocrinol. 8, 228-236. (2011).
3. Wild, S., et al. Diabetes Care 27, 1047-1053. (2004).
4. Kautzky-Willer, A., et al. Endocr. Rev. 37, 278-316. (2016).
5. Hoang-Yen Tran, D. et al. Int. J. Obes. (Lond.) (2016).
6. Mohamed, W. A. & Schaalan, M. F. Diabetol. Metab. Syndr. 10, 89. (2018).
7. Pound, L. D. et al. Diabetes 64, 4135-4147. (2015).
8. Musante, L. et al. J. Diabetes Res. 2015, 289734. (2015).
9. Shaw, L. & Wiedow, 0. Biochem. Soc. Trans. 39, 1450-1454. (2011).
10. Wang, J. et al. PLoS ONE 15, e0231796. (2020).
11. Paczesny, S. et al. Sci. Transl. Med. 2, 13ra12. (2010).
12. Sumi, Y. et al. Atherosclerosis 160, 31-39. (2002).
13. Butler, M. W. et al. J. Biol. Chem. 281, 34730-34735. (2006).
14. Henriksen, P. A. et al. J. Immunol. 172, 4535-4544 (2004).
15. Nickel, N. P. et al. Am. J. Respir. Crit. Care Med. 191, 1273-1286. (2015).
16. Bermudez-Humaran, L. G. et al. Microb. Cell Fact. 14, 26. (2015).
17. Galipeau, H. J. et al. Am. J. Gastroenterol. 109, 748-756. (2014).
18. Heydemann, A. J. Diabetes Res. 2016, 2902351. (2016).
19. Han, B. G. et al. Am. J. Physiol. Endocrinol. Metab. 295, E981-986. (2008).
20. Wang, C. Y. & Liao, J. K. Methods Mol. Biol. 821, 421-433 (2012).
21. King, A. J. Br. J. Pharmacol. 166, 877-894. (2012).
22. Pettersson, U. S., et al. PLoS ONE 7, e46057. (2012).
23. Fengler, V. H. et al. PLoS ONE 11, e0155163. (2016).
24. Winzell, M. S. & Ahren, B. Diabetes 53(Suppl 3), S215-219 (2004).
25. Ludgero-Correia, A. Jr., et al. Nutrition 28, 316-323. (2012).
26. Jequier, E. Ann. N.Y. Acad. Sci. 967, 379-388. (2002).
27. Mombaerts, P. et al. Cell 68, 869-877 (1992).
28. Montgomery, M. K. et al. Diabetologia 56, 1129-1139. (2013).
29. Robbins, P. D. & Morelli, A. E. Nat. Rev. Immunol. 14, 195-208. (2014).
30. Wen, C. et al. J. Extracell. Vesicles 6, 1400370. (2017).
31. Deiuliis, J. A. Int. J. Obes. (Lond.) 40, 88-101. (2016).
32. Arner, P. & Kulyte, A. Nat. Rev. Endocrinol. 11, 276-288. (2015).
33. Gallo, A., et al. PLoS ONE 7, e30679. (2012).
34. Deng, Z. B. et al. Diabetes 58, 2498-2505. (2009).
35. Kraus, N. A. et al. Adipocyte 5, 351-358. (2016).
36. Korner, A. et al. Biochem. Biophys. Res. Commun. 337, 540-550. (2005).
37. Christiaens, V., et al. Biochim. Biophys. Acta 949-956, 2012. (1820).
38. Wilson, C. G. et al. Endocrinology 157, 570-585. (2016).
39. Trak-Smayra, V. et al. Int. J. Exp. Pathol. 92, 413-421. (2011).
40. Savard, C. et al. Hepatology 57, 81-92. (2013).
41. Patel, M. V., et al. Am. J. Reprod. Immunol. 69, 463-474. (2013).
42. Hunt, K. K. et al. Breast Cancer Res. 15, R3. (2013).
43. Jo, A. & Mainous, A. G. 3rd. BMJ Open 8, e019200. (2018).
44. Guay, C. et al. Cell Metab. 29, 348.e346-361.e346. (2019).
45. Wong, N. et al. Endocrinology 152, 3690-3699. (2011).
46. O'Rourke, R. W. et al. Metabolism 61, 1152-1161. (2012).
47. Rocha, V. Z. et al. Circ. Res. 103, 467-476. (2008).
48. Nardelli, C. et al. Int. J. Obes. (Lond.) 38, 466-469. (2014).
49. Sun, X. et al. Circ. Res. 118, 810-821. (2016).
50. Fayyad-Kazan, H. et al. Hum. Immunol. 75, 677-685. (2014).
51. Lin, S., et al. Int. J. Obes. Relat. Metab. Disord. 24, 639-646 (2000).
52. Jager, J., et al. Endocrinology 148, 241-251. (2007).
53. Petit, V. et al. J. Lipid Res. 48, 278-287. (2007).
54. Ma, Y., et al. Sci. Rep. 6, 26325. (2016).
55. Melis, M. et al. PLoS ONE 14, e0211071. (2019).
56. Wieckowska, A. et al. Am. J. Gastroenterol. 103, 1372-1379. (2008).
57. Crespo, J. et al. Hepatology 34, 1158-1163. (2001).
58. Weisberg, S. P. et al. J. Clin. Investig. 112, 1796-1808. (2003).
59. Russo, L. & Lumeng, C. N. Immunology 155, 407-417. (2018).
60. Alam, S. R. et al. Heart 101, 1639-1645. (2015).
61. Wang, J. et al. Gastroenterology 154, 1737-1750. (2018).
62. Xu, C. et al. Sci. Rep. 7, 16351. (2017).
63. American Diabetes, A. Diabetes Care 42, S13-S28. (2019).
64. Karagiannides, I. et al. Physiol. Rep. 2, e00284. (2014).
65. Ostanin, D. V. et al. Am. J. Physiol. Gastrointest. Liver Physiol. 296, G135-146. (2009).
66. Kleiner, D. E. et al. Hepatology 41, 1313-1321. (2005).
67. Sideri, A. et al. Cell Mol. Gastroenterol. Hepatol. 1, 420-432. (2015).
68. Hing, T. C. et al. Gut 62, 1295-1305. (2013).

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A method of treating intestinal stricture in a subject having Crohn's disease, the method comprising administering elafin to the subject.

2. The method of claim 1, wherein a serum sample obtained from the subject has been assayed for elafin, and wherein the assay detects an elevated level of elafin relative to a control sample.

3. A method of inhibiting intestinal fibrosis, inflammatory bowel disease (IBD), metabolic disease, or obesity in a subject, the method comprising administering elafin to the subject.

4. The method of claim 1, 2 or 3, wherein the elafin is administered orally or subcutaneously.

5. The method of claim 1, 2 or 3, wherein the elafin is administered to the subject via an elafin-overexpressing vector.

6. The method of claim 5, wherein the vector is a bacterial or viral vector.

7. The method of claim 6, wherein the vector is a lactic acid bacterium.

8. The method of claim 1, 2 or 3, wherein the elafin is administered intracolonically.

9. The method of claim 4, wherein the elafin is administered via a slow release capsule.

10. The method of claim 2, wherein the assay is an immunoassay or a polymperase chain reaction (PCR) assay.

11. The method of claim 10, wherein the immunoassay is an enzyme linked immunosorbent assay (ELISA).

12. The method of claim 10, wherein the PCR is real time reverse transcriptase PCR (RT-PCR).

13. The method of claim 2, wherein the elevated level of elafin is greater than or equal to 8000 μg/ml.

14. The method of claim 2, further comprising determining a probability score, wherein the score comprises a serum elafin level in pg/ml and at least three clinical scores selected from the group consisting of: (1) age of the subject in years, (2) years of disease duration, (3) serum C-reactive protein (CRP) level in mg/L, (4) erythrocyte sedimentation rate (ESR) in mm/hour, (5) Harvey Bradshaw Index number (HBI), (6) number of inflammatory bowel disease related surgeries, (7) gender, (8) smoking status, (9) status of biologics (e.g., anti-TNF inhibitor) use, (10) status of steroid use, (11) status of immunomodulator use, (12) status of aminosalicylate use, and (13) presence of fistula.

15. The method of claim 14, wherein the probability score is determined using a machine learning algorithm.

16. The method of claim 14, wherein a probability score between 0 and 0.5 is indicative of absence of stricture, and a probability score of 0.51 to 1.0 is indicative of stricture.

17. The method of claim 16, wherein the algorithm is that available through Microsoft Azure Machine Learning Studio at gallery.cortanaintelligence.com/Experiment/Use-elafin-and-clinical-data-for-indicating-stricture-Predictive-Exp.

18. The method of claim 14, wherein the probability score further comprises one or more of the following clinical scores: (14) serum LL-37 level in ng/ml, (15) serum TGF-b1 level in pg/ml, (16) serum Cyr61 level in pg/ml.

19. The method of claim 3, wherein the inhibiting is for intestinal fibrosis.


SEQUENCE LISTING (2 sequences)

SEQ ID NO: 1 - protein, 117 aa, Homo sapiens
MRASSFLIVVVFLIAGTLVLEAAVTGVPVKGQDTVKGRVPFNGQDPVKGQVSVKGQDKVKAQEPVKGPVSTKPGSCPIILIRCAMLNPPNRCLKDTDCPGIKKCCEGSCGMACFVPQ

SEQ ID NO: 2 - protein, 57 aa, Homo sapiens
AQEPVKGPVSTKPGSCPIILIRCAMLNPPNRCLKDTDCPGIKKCCEGSCGMACFVPQ